(19) United States
(12) Patent Application Publication
(10) Pub. No.: US 2003/0149237 A1
(43) Pub. Date: Aug. 7, 2003

(54) Novel polynucleotides and polypeptides encoded thereby

(76) Inventors: Corine Vernet, North Branford, CT (US); Velizar Tchernev, Branford, CT (US); Meera Patturajan, Branford, CT (US); Uriel M. Malyankar, North Branford, CT (US); Vladimir Gusev, Madison, CT (US); John L. Herrmann, Guilford, CT (US); John R. MacDougall, Hamden, CT (US); Luca Rastelli, Guilford, CT (US); Haihong Zhong, Guilford, CT (US); Kimberly A. Spytek, New Haven, CT (US); Suresh Shenoy, Branford, CT (US); Valerie L. Gerlach, Branford, CT (US); Esha A. Gangolli, Madison, CT (US); David J. Stone, Guilford, CT (US); Glennda Smithson, Branford, CT (US)

Correspondence Address: MINTZ, LEVIN, COHN, FERRIS,, GLOVSKY and POPEO, P.C., One Financial Center, Boston, MA, 02111, US

(21) Appl. No.: 09/894,159

(22) Filed: Jun. 27, 2001

Related U.S. Application Data
(60) Provisional application No. 60/248,153, filed on Nov. 13, 2000.
(60) Provisional application No. 60/261,014, filed on Jan. 11, 2001.
(60) Provisional application No. 60/214,759, filed on Jun. 27, 2000.
(60) Provisional application No. 60/263,215, filed on Jan. 22, 2001.
(60) Provisional application No. 60/244,546, filed on Oct. 31, 2000.

(51) Int. Cl. C07K 1/00; C07K 14/00; C07K 17/00

(52) U.S. Cl. 530/350

(57) ABSTRACT

Disclosed herein are nucleic acid sequences that encode G-coupled protein-receptor related polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from Provisional Applications U.S. Ser. No. 60/214,759, filed Jun. 5, 27, 2000; U.S. Ser. No. 60/244,546, filed Oct. 31, 2000; U.S. Ser. No. 60/263,215, filed Jan. 22, 2001; U.S. Ser. No. 60/261,014, filed Jan. 11, 2001; and U.S. Ser. No. 60/248,153, filed Nov. 13, 2000, each of which is incorporated herein by reference in its entirety.

### FIELD OF THE INVENTION

The invention generally relates to nucleic acids and polypeptides encoded therefrom.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1a, NOV1b, NOV2, NOV3, NOV4, NOV5, NOV6a, NOV6b, NOV6c, NOV6d, NOV7a, NOV7b, an d NOV7c nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS:1, 3, 5, 7, 9, 11, 13, 15, 16, 17, 19, 21, 23, 25 and 27. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 18, 20, 22, 24, 26 and 28. The nucleic acid can be, for example, a genomic DNA fragment or a cDNA molecule that includes the nucleic acid sequence of any of SEQ ID NOS:1, 3, 5, 7, 9, 11, 13, 15, 16, 17, 19, 21, 23, 25 and 27.

Also included in the invention is an oligonucleotide, e.g., an oligonucleotide which includes at least 6 contiguous nucleotides of a NOVX nucleic acid (e.g., SEQ ID NOS:1, 3, 5, 7, 9, 11, 13, 15, 16, 17, 19, 21, 23, 25 and 27) or a complement of said oligonucleotide.

Also included in the invention are substantially purified NOVX polypeptides (SEQ ID NOS:2, 4, 6, 8, 10, 12, 14, 18, 20, 22, 24, 26 and 28). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., diabetes, metabolic disturbances associated with obesity, the metabolic syndrome X, anorexia, wasting disorders associated with chronic diseases, metabolic disorders, diabetes, obesity, infectious disease. anorexia, cancer-associated cachexia, cancer, neurodegenerative disorders, Alzheimer's Disease, Parkinson's Disorder, immune disorders, and hematopoietic disorders, or other disorders related to cell signal processing and metabolic pathway modulation. The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from: developmental diseases, MHCII and III diseases (immune diseases), taste and scent detectability Disorders, Burkitt's lymphoma, corticoneurogenic disease, signal transduction pathway disorders, Retinal diseases including those involving photoreception, Cell growth rate disorders; cell shape disorders, feeding disorders; control of feeding; potential obesity due to over-eating; potential disorders due to starvation (lack of appetite), noninsulin-dependent diabetes mellitus (NIDDM 1), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy (DRPLA) Hypophosphatemic rickets, autosomal dominant (2) Acrocallosal syndrome and dyskinesias, such as Huntington's disease or Gilles de la Tourette syndrome and/or other pathologies and disorders of the like.

The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders.

The invention further includes a method for screening for a modulator of disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders or other disorders related to cell signal processing and metabolic pathway modulation. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders or other disorders related to cell signal processing and metabolic pathway modulation by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next. the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their polypeptides. The sequences are collectively referred to as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table 8 provides a summary of the NOVX nucleic acids and their encoded polypeptides.

TABLE 8
Sequences and Corresponding SEQ ID Numbers

| | | SEQ ID | | |
|---|---|---|---|---|
| | | NO | | |
| NOVX | | (nucleic | SEQ ID NO | |
| Assignment | Internal Identification | acid) | (polypeptide) | Homology |
| 1a | 20421338.0.44 | 1 | 2 | Irregular Chiasm C Roughest-Like |
| | (CG51373-02) | | | Protein |
| 1b | 20421338.0.30 | 3 | 4 | Irregular Chiasm C Roughest-Like |
| | (CG51373-01) | | | Protein |
| 2 | 21424344.9.6 | 5 | 6 | LDL Receptor Related Protein |
| 3 | B80173.9.32 | 7 | 8 | Small lnducible Cytokine-Like |
| | | | | Protein |
| 4 | 83614984.0.5 | 9 | 10 | Cell Cycle and Proliferation |
| | (CG53006-01) | | | Protein-Like Protein |
| 5 | 34405797.0.15 | 11 | 12 | Cadherin-Like Protein |
| 6a1 | 5603288.0.2.0_da1 | 13 | 14 | Lysozyme C-1 Precursor-Like |
| | | | | Protein |
| 6a2 | CG52754-03 | 15 | | Lysozyme C-1 Precursor-Like |
| | | | | Protein |
| 6a3 | 30412306_0_100_da1 | 16 | | Lysozyme C-1 Precursor-Like |
| | | | | Protein |
| 6b | 5603288.0.1 | 17 | 18 | Lysozyme C-1 Precursor-Like |
| | (CG52754-01) | | | Protein |
| 6c | CG52754-02 | 19 | 20 | Lysozyme C-1 Precursor-Like |
| | | | | Protein |
| 6d | 30412306_0_100_da1 | 21 | 22 | Lysozyme C-1 Precursor-Like |
| | (B) | | | Protein |
| 7a | CG51373-10 | 23 | 24 | IgG-Like Protein |
| 7b | 20421338_1 | 25 | 26 | IgG-Like Protein |
| | (CG51373-03) | | | |
| 7c | 20421338_2 | 27 | 28 | IgG-Like Protein |
| | (CG51373-04) | | | |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

For example, NOV1 is homologous to members of Irregular Chiasm C Roughest family of proteins that are important cell adhesion molecules and members of the immunoglobulin superfamily. Thus, the NOV1 nucleic acids, polypeptides, antibodies and related compounds according to the invention will be useful in therapeutic and diagnostic applications in disorders characterized by cell migration, invasion and tumor metastasis, e.g., lymphoproliferative disease.

Also, NOV2 is homologous to low density lipoprotein (LDL) receptor related protein family. Thus NOV2 may function similarly to other members of the LDL receptor family. Consequently, the NOV2 nucleic acids, polypeptides, antibodies and related compounds according to the invention will be useful in therapeutic and diagnostic applications in disorders characterized by e.g., high levels of cholesterol-rich LDL in the plasma, eg., familial hypercholesterolemia.

Further, NOV3 is homologous to a family of small inducible cytokine proteins that include GRO proteins and Interleukin-8 (IL-8). Thus, the NOV3 nucleic acids and polypeptides, antibodies and related compounds according to the invention will be useful in therapeutic applications in various disorders involving GRO proteins, IL-8 and/or other members of the same family. Specific examples of these disorders include, for example, Crohn's disease, inflammatory bowel disease, ulcerative colitis and various types of cancers.

Also, NOV4 is homologous to the cell cycle and proliferative proteins important in cell cycle regulation and cell proliferation. Thus, NOV4 nucleic acids, polypeptides, antibodies and related compounds according to the invention will be useful, for example, in therapeutic and diagnostic applications in various immune, developmental and cell signaling disorders and cell proliferative disorders including cancer.

Additionally, NOV5 is homologous to the cadherin family of proteins. Thus NOV5 nucleic acids, polypeptides, antibodies and related compounds according to the invention will be useful in treating a variety of conditions, including, e.g., immune deficiencies and disorders, viral, bacterial and other infections, and cell proliferative disorders.

Further, NOV6 is homologous to the lysozyme C-1 family of proteins. Thus, NOV6 nucleic acids, polypeptides, antibodies and related compounds according to the invention will be useful in treating a variety of conditions, including, e.g., bacterial, fungal, protozoal and viral infections, amyloidosis, blood disorders, salivitory disorders, digestive disorders, oral immunologic disorders, poor oral health, inflammatory processes, muscle, bone and tendon disorders, and/or other pathologies and disorders of the like.

Finally, NOV7 is homologous to members of IgG-like proteins that are important protease inhibitors and cancer antigens. Thus, the NOV7 nucleic acids, polypeptides, antibodies and related compounds according to the invention will be useful in therapeutic and diagnostic applications in disorders (e.g., proliferative disorders) characterized by protease inhibition and carcinoma.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

NOV1 includes a family of two similar nucleic acids and two similar proteins disclosed below. They are novel members of the Ig superfamily of proteins, as demonstrated by the presence of identifiable Ig domains contained within NOV1.

NOV1a

The disclosed NOV1a nucleic acid of 3464 nucleotides (also referred to as 20421338.0.44, or CG51373-02) is shown in Table 1A. An ORF begins with an ATG initiation codon at nucleotides 1-3 and ends with a stop codon at nucleotides 2524-2526. A putative untranslated region downstream from the termination codon is underlined in Table 1A, and the start and stop codons are in bold letters.

TABLE 1A

NOV1a Nucleotide Sequence

(SEQ ID NO:1)

ATGCATTTGACTCTGGAAGTCTTAAACCATGGCCCCTTCCCTCTAAACCTTTCCTCCATTGCTTACAATCATGGAACTGT
GTTTGGCCACTGGAAGAATAACGTCACTCGGGAAACGCTGGTGAAAGTAAAAGATGCTGAAGATCAGTTGGGTGCACGAG
TGGGTTACATCGAACTGGATCTCAACAGCGGGAAGGAAACATTTCTGGTGAATGAGGAGGCAACGGGCGAGACCTCAGGA
GACAATGTTGTTCATTCTAGGAATCTGTCTCAGACAATCTTCATCACCCGGAAACGATGGGAGGGGACCCAGACCCGCTT
CAGCCAGGAGCCAGCTGACCAGACGGTGGTGGCTGGACAGCGGGCCGTGCTCCCCTGTGTGCTGCTCAACTACTCTGGAA
TCCGCAGACGCTGGGCAGTACAACCTGGAGATCACAGATGCTGAGCTCTCTGACGACGCCTCTTACGAGTGCCAGGCCAC
GGAGGCCGCCCTGCGCTCTCGGCGGGCCAAACTCACCGTGCTCATCCCCCCAGAGGACACCAGGATTGACGGAGGCCCTG
TGATTCTACTGCAGGCAGGCACCCCCCACAACCTCACATGCCGGGCCTTCAATGCGAAGCCTGCTGCCACCATCATCTGG
TTCCGGGACGGGACGCAGCAGGAGGGCGCTGTGGCCAGCACGGAATTGCTGAAGGATGGGAAGAGGGAGACCACCGTGAG
CCAACTGCTTATTAACCCCACGGACCTGGACATAGGGCGTGTCTTCACTTGCCGAAGCATGAACGAAGCCATCCCTAGTG
GCAAGGAGACTTCCATCGAGCTGGATGTGCACCACCCTCCTACAGTGACCCTGTCCATTGAGCCACAGACGGTGCAGGAG
GGTGAGCGTGTTGTCTTTACCTTGCCAGGCCACAGCCAACCCCGAGATCTTGGGCTACAGGTGGGCCAAGGGGGTTTCTT
GATTGAAGACGCCCACGAGAGTCGCTATGAGACAAATGTGGATTATTCCTTTTTCACGGAGCCTGTGTCTTGTGAGGTTC
ACAACAAAGTGGGAAGCACCAATGTCAGCACTTTAGTAAATGTCCACTTTGCTCCCCGGATTGTAGTTGACCCCAAACCC
ACAACCACAGACATTGGCTCTGATGTGACCCTTACCTGTGTCTGGGTTGGGAATCCCCCCCTCACTCTCACCTGGACCAA
AAAGGACTCAAATATGGTCCTGAGTAACAGCAACCAGCTGCTGCTGAAGTCGGTGACTCAGGCAGACGCTGGCACCTACA
CCTGCCGGGCCATCGTGCCTCGAATCGGAGTGGCTGAGCGGGAGGTGCCGCTCTATGTGAACGGGCCCCCCATCATCTCC
AGTGAGGCAGTGCAGTATGCTGTGAGGGGTGACGGTGGCAAGGTGGAGTGTTTCATTGGGAGCACACCACCCCCAGACCG
CATAGCATGGGCCTGGAAGGAGAACTTCTTGGAGGTGGGGACCCTGGAACGCTATACAGTGGAGAGGACCAACTCAGGCA
GTGGGGTGCTATCCACGCTCACCATCAACAATGTCATGGAGGCCGACTTTCAGACTCACTACAACTGCACCGCCTGGAAC
AGCTTCGGGCCAGGCACAGCCATCATCCAGCTGGAAGAGCGAGAGGTGTTACCTGTGGGCATCATAGCTGGGGCCACCAT
CGGCGCGAGCATCCTGCTCATCTTCTTCTTCATCGCCTTGGTATTCTTCCTCTACCGGCGCCGCAAAGGCAGTCGCAAAG
ACGTGACCCTGAGGAAGCTGGATATCAAGGTGGAGACAGTGAACCGAGAGCCACTTACGATGCATTCTGACCGGGAGGAT
GACACCGCCAGCGTCTCCACAGCAACCCGGGTCATGAAGGCCATCTACTCGTCGTTTAAGGATGATGTGGATCTGAAGCA
GGACCTGCGCTGCGACACCATCGACACCCGGGAGGAGTATGAGATGAAGGACCCCACCAATGGCTACTACAACGTGCGTG
CCCATGAAGACCGCCCGTCTTCCAGGGCAGTGCTCTATGCTGACTACCGTGCCCCTGGCCCTGCCCGCTTCGACGGCCGC
CCCTCATCCCGTCTCTCCCACTCCAGCGGCTATGCCCAGCTCAACACCTATAGCCGGGGCCCTGCCTCTGACTATGGCCC
TGAGCCCACACCCCCTGGCCCTGCTGCCCCAGCTGGCACTGACACAACCAGCCAGCTGTCCTACGAGAACTATGAGAAGT
TCAACTCCCATCCCTTCCCTGGGGCAGCTGGGTACCCCACCTACCGACTGGGCTACCCCCAGGCCCCACCCTCTGGCCTG
GAGCGGACCCCATATGAGGCGTATGACCCCATTGGCAAGTACGCCACAGCCACTCGATTCTCCTACACCTCCCAGCACTC
GGACTACGGCCAGCGATTCCAGCAGCGCATGCAGACTCACGTGTAGGGGCCAGAGCCTGGCTGGGGCATCTCTGCGGGGC
AGAGGAGAAGGCTTTCGCAGCTGTTCCCTGATATTCAGGGACATTGCTCATTGCTCCCTTCTCGGACCAGCCTTCTTCCT
CCCACCATGGCAGGTGGGGAGCAGGTCTCCCAGAGACACCCCGTCCCGAGGATGGTGCTCTGTGCATGCCCCAGCCTCCT
GGGCCTGCCCTTCCCTCTTCTTCGGGAGGATGTGTCTCTTCTGACCTGCACTCTTGCCTGACCCTAGAATGGGGACAGGG
AAAGTGAAGGTTAGGGAAAGCAGAGGGGGGCACTTTTTAGCATTCCCTTTCTATCCCACCCCTCTGATCTCCCATAAGTG
GAAATGGGGGTACCCAGGGATGGGCAGGCTTTGGCCTAGGGACATGAAGTATGGGAGTGGGTGGCTGTGGCACAGACAGG
TGGAAAACGGGATAGCCTGGCCAGTCCCTCTGTTGTCTGCATTCGTGCCCTGGGTGCCTCTCTCCTTCCTCAGGGTACTG
CAGAAGGGAGCGAACAGGGTACTGTTCGCTCTTGTCTACAGAACAGCCCTGGCACTGCATTCAAATCCAGTCTTCATTCA
GCTGGGATCAAAATGCCAGTCACCTTGGCTACCCACTGTGGACAGCTGTCTGTCAGCATGCAGAGGGATCCAGGAATCCC
CCCGGCAGCACGGCCCGCTTTCCTTCTCCTCCATGCTGGGCCAGCCAGATAAGTCAGGGTCCTGGTGGAGAAAGAAAGGC
TAGGACCATGTCCTCATTGACCCAGATACTGCTGTGTGCTGCACAGCAGTGAACCAACACTAGAGGGAGCCACACAAGCC
TCCTCTCCCCAGTCTGCCCCACTTCCTGGCTTTAACTCTTGAGCTGGTTTGGGGAGTGGTGAGGTAGGGGTGGGGGTGCT
GTAGGCTCTTTTTCAAAAAAAAAC

The NOV1a protein encoded by SEQ ID NO:2 has 841 amino acid residues and is presented using the one-letter code in Table 1B. The Psort profile for NOV1a predicts that this sequence likely has no signal peptide and is likely to be localized at the plasma membrane with a certainty of 0.7000; to the microbody (peroxisome) (certainty=0.3661); endoplasmic reticulum (membrane) (certainty=0.2000); and mitochondrial inner membrane (certainty=0.1000). Because of the presence of identifiable Ig domains, the NOV1a protein is a member of the Ig superfamily.

TABLE 1B

Encoded NOV1a protein sequence

(SEQ ID NO:2)

MHLTLEVLNHGPFPLNLSSIAYNHGTVFGHWKNNVTRETLVKVKDAEDQLGARVGYIELDLNSGKETFLVNEEATGETSG
DNVVHSRNLSQTIFITRKRWEGTQTRFSQEPADQTVVAGQRAVLPCVLLNYSGIVQWTKDGLALGMGQALKAWPRYRVVG
SADAGQYNLEITDAELSDDASYECQATEAALRSRRAKLTVLIPPEDTRIDGGPVILLQAGTPHNLTCRAFNAKPAATIIW
FRDGTQQEGAVASTELLKDGKRETTVSQLLINPTDLDIGRVFTCRSMNEAIPSGKETSIELDVHHPPTVTLSIEPQTVQE
GERVVFTCQATANPEILGYRWAKGGFLIEDAHESRYETNVDYSFFTEPVSCEVHNKVGSTNVSTLVNVHFAPRIVVDPKP
TTTDIGSDVTLTCVWVGNPPLTLTWTKKDSNMVLSNSNQLLLKSVTQADAGTYTCRAIVPRIGVAEREVPLYVNGPPIIS
SEAVQYAVRGDGGKVECFIGSTPPPDRIAWAWKENFLEVGTLERYTVERTNSGSGVLSTLTINNVMEADFQTHYNCTAWN
SFGPGTAIIQLEEREVLPVGIIAGATIGASILLIFFFIALVFFLYRRRKGSRKDVTLRKLDIKVETVNREPLTMHSDRED
DTASVSTATRVMKAIYSSFKDDVDLKQDLRCDTIDTREEYEMKDPTNGYYNVRAHEDRPSSRAVLYADYRAPGPARFDGR
PSSRLSHSSGYAQLNTYSRGPASDYGPEPTPPGPAAPAGTDTTSQLSYENYEKFNSHPFPGAAGYPTYRLGYPQAPPSGL
ERTPYEAYDPIGKYATATRFSYTSQHSDYGQRFQQRMQTHV

The disclosed amino acid sequence for NOV1a has 410/410 (1 00%) identical to a 410 amino acid Homo sapiens irregular chiasm c-roughest protein precursor (ACC:BAA91850) (cDNA FLJ10845 FIS, Clone NT2RP4001372) (score=2161 (760.7 bits); E=8.3e-224). The disclosed NOV1a amino acid sequence also has 89/291 (30%) identical and 139/291 (47%) positives with a 764 amino acid ACC:A08180 Irregular Chiasm C-Roughest Protein Precursor (IRREC PROTEIN) from Drosophila melanogaster (score=351 (123.6 bits); E=3.8e-59).

The roughest-irregular chiasm C protein is a cell adhesion molecule that is a transmembrane glycoprotein of the immunoglobulin superfamily involved in several important developmental processes in Drosophila. These include axonal pathfinding in the optic lobe and programmed cell death and pigment cell differentiation in the pupal retina. See Moda et al., An Acad Bras Cienc 72(3):381-88 (2000). Additionally, this protein plays a role in patterning sense organs on the Drosophila antenna. See Venugopala Reddy et al., Dev Genes Evol 209(10):581-91 (1999). Pattern formation in the developing Drosophila retina involves the elimination of excess cells between ommatidia and the differentiation of the remaining cells into secondary and tertiary pigment cells. See Reiter et al., Development 122(6):1931-40 (1996). Irregular chiasmC-roughest protein is essential for correct sorting of cell-cell contacts in the pupal retina. See id.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the IIT BLAST analysis, matched the Query IIT sequence purely by chance is the E value. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences. Blasting is performed against public nucleotide databases such as GenBank databases and the GeneSeq patent database. For example, BLASTX searching is performed against public protein databases, which include GenBank databases, SwissProt, PDB and PIR.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXXX”). Low-complexity regions can result in high scores that reflect compositional bias rather than significant position-by-position alignment. Wootton and Federhen, Methods Enzymol 266:554-571, 1996.

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

Possible SNPs for NOV1a include those found in Table 1C.

TABLE 1C
cSNPs for NOV1a

| Base | | | |
|---|---|---|---|
| Position of | | | |
| cSNP | Wild Type | Variant | Amino Acid Change |
| 2951 | C | A | None |
| 1979 | A | T | Lys−>Met |
| 1013 | G | A | Gly−>Asp |
| 1002 | C | G | None |
| 740 | A | G | Gln−>Arg |
| 557 | C | T | Ala−>Val |
| 744 | C | A | None |
| 798 | G | A | None |
| 852 | C | T | None |

NOV1b

The nucleotide sequence for NOV1b (20421338.0.30 or CG51373-01) (1230 bp, SEQ ID NO:3) is presented in Table 1D. An open reading frame was identified beginning at nucleotides 1-3 and ending at nucleotides 1003-1005. The start and stop codons of the open reading frame are highlighted in bold type, and putative untranslated regions are underlined.

TABLE 1D

NOV1b Nucleotide Sequence

(SEQ ID NO:3)

ATGCATTTGACTCTGGAAGTCTTAAACCATGGCCCCTTCCCTCTAAACCTTTCCTCCATTGCTTACAATCATGGAACTGT
GTTTGGCCACTGGAAGAATAACGTCACTCGGGAAACGCTGGTGAAAGTAAAAGATGCTGAAGATCAGTTGGGTGCACGAG
TGGGTTACATCGAACTGGATCTCAACAGCGGGAAGGAAACATTTCTGGTGAATGAGGAGGCAACGGGCGAGACCTCAGGA
GACAATGTTGTTCATTCTAGGAATCTGTCTCAGACAATCTTCATCACCCGGAAACGATGGGAGGGGACCCAGACCCGCTT
CAGCCAGGAGCCAGCTGACCAGACGGTGGTGGCTGGACAGCGGGCCGTGCTCCCCTGTGTGCTGCTCAACTACTCTGGAA
TTGTGCAATGGACCAAGGACGGGCTGGCCCTGGGCATGGGCCAGGCCCTCAAAGCCTGGCCACGGTACCGGGTTGTGGGC
TCCGCAGACGCTGGGCAGTACAACCTGGAGATCACAGATGCTGAGCTCTCTGACGACGCCTCTTACGAGTGCCAGGCCAC
GGAGGCCGCCCTGCGCTCTCGGCGGGCCAAACTCACCGTGCTCATCCCCCCAGAGGACACCAGGATTGACGGAGGCCCTG
TGATTCTACTGCAGGCAGGCACCCCCCACAACCTCACATGCCGGGCCTTCAATGCGAAGCCTGCTGCCACCATCATCTGG
TTCCGGGACGGGACGCAGCAGGAGGGCGCTGTGGCCAGCACGGAATTGCTGAAGGATGGGAAGAGGGAGACCACCGTGAG
CCAACTGCTTATTAACCCCACGGACCTGGACATAGGGCGTGTCTTCACTTGCCGAAGCATGAACGAAGCCATCCCTAGTG
GCAAGGAGACTTCCATCGAGCTGGATGTGCACCGTGAGTGGGCTGGGGGGAGCAGTCTGGAGCAGGGGGGTGGAAGAAGG
GGTGTGTTTGAGAAGCACACTCTTAGTTTGAGAAACACAAACTAAGAGTCCCCCTATGGTCCCCAGGACAAACGCTTGCC
TTCTTCACATCTTTCATTCCCTGGATTGAACCATGGGGACTAAGGGCTGGTAGAGCATTGGCTGTGGAGTCAGGCAGTCC
CCAGGTCTAAACCAGCCTGTTATTAGTCAATGGTTTACACTCTCTGGGCCTCGGTTTCCAGTTCTGTATACTGTATATTG
CAAAAGATAAAATACTGGCCTACAGCCCCA

The encoded NOV1b protein is presented in Table 1E. The disclosed protein is 334 amino acids long and is denoted by SEQ ID NO:4. The Psort profile for NOV1b predicts that this sequence likely has no signal peptide and is likely to be localized in the cytoplasm with a certainty of 0.4500; microbody (peroxisome) with a certainty of 0.3235; lysosome (lumen) with a certainty of 0.2075; and mitochondrial matrix space with a certainty of 0.1000. The disclosed NOV1b protein is expressed in the lymph node, ovary, and adrenal gland. The disclosed NOV1b protein is a member of the Ig superfamily, demonstrated by its homology to identifiable Ig domains contained therein.

NOV1b is likely a plasma membrane Type 1b membrane protein. SAGE analysis indicates that this gene is upregulated by EGFr.

TABLE 1E

Encoded NOV1b protein sequence

(SEQ ID NO:4)

MHLTLEVLNHGPFPLNLSSIAYNHGTVFGHWKNNVTRETLVKVKDAEDQLGARVGYIELDLNSGKETFLVNEEATGETSG
DNVVHSRNLSQTIFITRKRWEGTQTRFSQEPADQTVVAGQRAVLPCVLLNYSGIVQWTKDGLALGMGQALKAWPRYRV
VGSADAGQYNLEITDAELSDDASYECQATEAALRSRRAKLTVLIPPEDTRDGGPVILLQAGTPHNLTCRAFNAKPAATIIW
FRDGTQQEGAVASTELLKDGKRETTVSQLLINPTDLDIGRVFTCRSMNEAIPSGKETSIELDVHREWAGGSSLEQGGGRR
GVFEKHTLSLRNTN

The disclosed NOV1b amino acid sequence has 77/205 (37%) identical and 106/205 (51%) with a 764 amino acid ACC:18180 Irregular Chiasm C-Roughest Protein Precursor (IRREC PROTEIN) from Drosophila melanogaster (score=326 (114.8 bits); E=2.3e-28). Additionally, the disclosed NOV1b protein has 59/199 (29%) identical and 93/199 (46%) positives with a 1241 amino acid ACC:O60500 Nephrin from Homo sapiens. As noted above, the Irregular Chiasm C-Roughest Protein is an adhesion molecule that is a member of the immunoglobulin superfamily. Likewise, nephrin is a putative member of the immunoglobulin of cell adhesion molecules. See Kestilia et al., Molec. Cell 1:575-82 (1998);OMIM 602716. Nephrin contains a transmembrane domain, eight Ig-like modules, and one fibronectin III-like module. See id. This protein has been shown to be specifically expresses in renal glomeruli, and it plays a crucial role in the development or function of the kidney filtration barrier. Putaala et al., Hum. Molec. Genet. 10:1-8 (2001) generated a mouse model for congenital nephritic syndrome by inactivating the nephrin gene in embryonic stem cell by homologous recombination.

Possible cSNPs for NOV1b are shown in Table 1F.

TABLE 1F
cSNPs for NOV1b

| Base | | | |
|---|---|---|---|
| Position of | | | |
| cSNP | Wild Type | Variant | Amino Acid Change |
| 2866 | C | A | None |
| 1894 | A | T | Lys−>Met |
| 928 | G | A | Gly−>Asp |
| 917 | C | G | None |
| 655 | A | G | Gln−>Arg |
| 472 | C | T | Ala−>Val |
| 659 | 0 | A | None |
| 713 | G | A | none |
| 767 | C | T | none |

Table 1G shows a comparison of the protein sequences of NOV1a and NOV1b.

[image]

[image]

Other BLAST results include sequences from the Patp database, which is a proprietary database that contains sequences published in patents and patent publications. Patp results for NOV1 include those listed in Table 1H and 1I.

Patp results for NOV1a include those listed in Table 1H.

TABLE 1H
Patp alignments of NOV1a

| | | | Smallest |
|---|---|---|---|
| | | | Sum |
| | Reading | High | Prob |
| | Frame | Score | P(N) |
| patp:AAB41021 Human ORFX | +1 | 654 | 2.0e-62 |
| polypeptide (126 aa) | | | |

Patp results for NOV1b include those listed in Table 1I:

TABLE 1I
Patp alignments of NOV1b

| | | | Smallest |
|---|---|---|---|
| | | | Sum |
| | Reading | High | Prob |
| | Frame | Score | P(N) |
| patp:AAB41021 Human ORFX | +2 | 486 | 1.6e-45 |
| polypeptide (126 aa) | | | |

For example, a BLAST against ORF785, a 126 amino acid human ORFX from Homo sapiens produced 126/126 (100%) identity and 126/126 (100%) positives (E=2.0e-62) with NOV1a. WO00/58473. Additionally, a BLAST against ORF785, produced 126/126 (100%) identity and 126/126 (100%) positives (E=1.6e-45) with NOV1b.

Unless specifically addressed as NOV1a or NOV1b any reference to NOV1 is assumed to encompass all variants. Residue differences between any NOVX variant sequences herein are written to show the residue in the “a” variant and the residue position with respect to the “a” variant. NOV residues in all following sequence alignments that differ between the individual NOV variants are highlighted with a box and marked with the (o) symbol above the variant residue in all alignments herein.

The disclosed NOV1 protein has good identity with a number of proteins within the Ig superfamily. The identity information used for ClustalW analysis is presented in Table 1J.

TABLE 1J
BLAST results for NOV1

| Gene Index/ | Protein/ | Length | Identity | Positives | |
|---|---|---|---|---|---|
| Identifier | Organism | (aa) | (%) | (%) | Expect |
| gi\|10434261\|dbj\| | unnamed protien | 571 | 555/571 | 555/571 | 0.0 |
| BAB14192.1\| | product | | (97%) | (97%) | |
| (AK022708) | Homo sapiens | | | Gaps = | |
| | | | | 16/571 | |
| | | | | (2%) | |
| gi\|8922705\|ref\| | hypothetical | 410 | 410/410 | 410/410 | 0.0 |
| NP_060710.1\|; | protein FLJ10845 | | (100%) | (100%) | |
| gi\|7023134\|dbj\| | Homo sapiens; | | | | |
| BAA91850.1\| | unnamed protein | | | | |
| (AK001707) | product | | | | |
| | Homo sapiens | | | | |
| gi\|14017951\|dbj\| | KIAA1867 protein | 779 | 317/654 | 416/654 | e−170 |
| BAB47496.1\| | Homo sapiens | | (48%) | (63%) | |
| (AB058770) | | | | Gaps = | |
| | | | | 25/654 | |
| | | | | (3%) | |
| gi\|13639054\|ref\| | hypothetical | 296 | 296/296 | 296/296 | e−144 |
| XP_002177.3\| | protein FLJ10845 | | (100%) | (100%) | |
| | Homo sapiens | | | | |
| gi\|9255755\|gb\| | KIRRE | 959 | 164/488 | 238/488 | 6e−65 |
| AAF86308.1\| | Drosophila | | (33%) | (48%) | |
| AF196553_1 | melanogaster; | | | Gaps = | |
| (AF196553); | dumbfounded | | | 33/488 | |
| gi\|12043535\|emb\| | Drosophila | | | (6%) | |
| CAB96574.2 | melanogaster | | | | |
| (AJ289882) | | | | | |

This information is presented graphically in the multiple sequence alignment given in Table 1J (with NOV1a being shown on line 1) as a ClustalW analysis comparing NOV1 with related protein sequences.

In all ClustalW analyses herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function.

[image]

[image]

[image]

NOV1 has been localized to chromosome 1. A BLAST of the NOV1 nucleic acids against the 154,998 bp Homo sapiens chromosome 1 clone RPI11-444M10 (acc:AL139010.8) yielded 1375/1414 (97%) identical and 1375/1414 (97%) positives (E=0.0; strand=minus/plus) from nucleotides 125669 to 127081. Likewise, NOV1 had 249/276 (90%) identical and 249/276 (90%) positives (E=1.8e-193; strand=minus/plus) from nucleotides 16632 to 16909 of this chromosome 1 clone; 172/189 (91%) identical and 172/189 (91%) positives (E=1.8e-193; strand =minus/plus) from nucleotides 20136 to 20324; 159/169 (94%) identical and 159/169 (94%) positives (E=1.8e-193; strand=minus/plus) from nucleotides 111311 to 111340; 140/140 (100%) identical and 140/140 (100%) positives (E=0.0; strand=minus/plus) from nucleotides 127963 to 128102; 133/135 (98%) identical and 133/135 (98%) positives (E=1.8e-193; strand=minus/plus) from nucleotides 19837 to 19971; 129/129 (100%) identical and 129/129 (100%) positives (E=1.8e-193; strand=minus/plus) from nucleotides 139891 to 139958; and 94/95 (98%) identical and 94/95 (98%) positives (E=1.8e-193; strand=minus/plus) from nucleotides 20942 to 21035. Additionally, a BLAST of NOV1 against the 195115 bp Homo sapiens chromosome 1 clone RPI1-404013 (acc:AL1138899.10) produced 63165 (96%) identical and 63/65 (96%) positives (E=0.0047; strand=plus/plus) from nucleotides 90500 to 90564 of this chromosome 1 clone; 1408/1415 (99%) identical and 1408/1415 (99%) positives (E=0.0; strand=minus/plus) from nucleotides 164149 to 165563; 162/164 (98%) identical and 162/164 (98%) positives (E=0.0; strand=minus/plus) from nucleotides 174892 to 175055; 152/152 (100%) identical and 152/152 (100%) positives (E=0.0; strand=minus/plus) from nucleotides 172726 to 172877; 159/168 (94%) identical and 159/168 (94%) positives (E=0.0; strand=minus/plus) from nucleotides 183193 to 183360; 140/140 (100%) identical and 140/140 (100%) positives (E=0.0; strand=minus/plus) from nucleotides 16644. to 166582; and 129/129 (100%) identical and 129/129 (100%) positives (E-0.0; strand=minus/plus) from nucleotides 170576to 170704.

The presence of identifiable domains in NOV1 was determined by searches using algorithms such as PROSITE, Blocks, Pfam, ProDomain, Prints and then determining the Interpro number by crossing the domain match (or numbers) using the Interpro website (http:www.ebi.ac.uk/interpro/).

DOMAIN results for NOV1 were collected from the Conserved Domain Database (CDD) with Reverse Position Specific BLAST. This BLAST samples domains found in the Smart and Pfam collections. The results are listed in Table 1K with the statistics and domain description. The presence of these identifiable domains is shown in Tables 1L-1P. For Tables 1L-1P, and all successive DOMAIN sequence alignments, fully conserved single residues are indicated by black shading and “strong” semi-conserved residues are indicated by grey shading. The “strong” group of conserved amino acid residues may be any one of the following groups of amino acids: STA, NEQK, NHQK, NDEQ, QHRK, MILV, MILF, HY, FYW.

TABLE 1K
DOMAIN results for NOV1

| | | Score | E |
|---|---|---|---|
| PSSMs producing significant alignments: | | (bits) | value |
| gnl\|Smart\|smart00408 | IGc2, Immunoglobulin C-2 Type | 53.9 | 3e-08 |
| gnl\|Smart\|smart00408 | IGc2, Imnmnoglobulin C-2 Type | 35.8 | 0.009 |
| gnl\|Smart\|smart00409 | IG, Immunoglobulin | 53.5 | 4e-08 |
| gnl\|Smart\|smart00409 | IG, Immunoglobulin | 49.3 | 8e-07 |
| gnl\|Smart\|smart00409 | IG, Immunoglobulin | 43.5 | 5e-05 |
| gnl\|Smart\|smart00409 | IG, Immunoglobulin | 39.7 | 7e-04 |
| gnl\|Pfam\|pfam00047 | Ig, Immunoglobulin domain | 38.1 | 0.002 |
| gnl\|Pfam\|pfam00047 | Ig, Immunoglobulin domain | 37.4 | 0.003 |
| gnl\|Pfam\|pfam00047 | Ig, Immunoglobulin domain | 37.4 | 0.003 |

[image]

[image]

NOV1 is a member of the immunoglobulin (Ig) superfamily. Members of this superfamily have a variety of functions, but all appear to play a role in cell recognition and the regulation of cell behavior. See OMIM entries 300137 and 147100. While constructing a YAC/STS map of the human X chromosome, Mazzarella et al., Genomics 48: 157-162 (1998) (PubMed ID: 9521868) identified a region that was highly conserved between the human and hamster genomes. Using a PCR-based approach, they isolated cDNAs corresponding to this region from a teratocarcinoma cell line library. The cDNAs encoded a predicted 1,327-amino acid protein that was designated ‘immunoglobulin superfamily member 1’ (IGSF1) because it contained 12 Ig-like domains of the C2 (constant region type 2) type. In addition, IGSF1 has a signal sequence and a potential transmembrane domain. Northern blot analysis revealed that IGSF1 was expressed as a 4.7-kb mRNA in many of the tissues tested, with the highest expression in pancreas, testis, and fetal liver. Additional 2.8- and 5.5-kb transcripts were observed in heart and testis, respectively. Independently, Nagase et al., DNA Res. 4: 141-150 (1997) (PubMed ID: 9205841) isolated a human brain cDNA (GenBank GENBANK AB002362) encoding IGSF1. Mazzarella et al. (1998) reported that the IGSF1 gene is located 0.5 Mb proximal to HDGF on Xq25, and is transcribed from centromere to telomere. Using a computer mapping approach Frattini et al., Genomics 38: 87-91 (1996) (PubMed ID : 8954785)), Frattini et al. (1998) mapped the IGSF1 gene to Xq25. See Frattini et al., Gene 214: 1-6 (1998) (PubMed ID:9729118).

In addition, Frattini et al. (1998) identified the IGDC1 gene (GenBank GENBANK Y10523), which encodes a member of the immunoglobulin-like domain-containing molecule superfamily. The 1,336-amino acid IGDC1 protein contains 12 Ig-like domains in 2 clusters of 5 and 7 motifs, which are followed by a linker segment, and a transmembrane domain and a cytoplasmic region, respectively. The IGDC1 gene is conserved in mammals and is expressed in muscle, heart, brain, testis, and pancreas as transcripts of different lengths, suggesting that it is subjected to alternative splicing. The IGDC1 gene contains 19 exons distributed along approximately 20 kb; each Ig-like domain is encoded by a discrete exon which constitutes, either singly or multiply, the unit of repeated genomic duplications. The function of this gene was unknown. In spite of its homology to natural killer (NK) cell inhibitory receptors, Frattini et al. (1998) were unable to demonstrate any expression of IGDC1 in purified NK cell populations or cell lines. This sequence similarity was intriguing, as the IGDC1 gene could have been involved in the pathogenesis of Xq25-linked lymphoproliferative disease (LYP), which presents with a defect in NK cell function.

The IGDC1 gene was recently identified by a computer-based approach (Frattini et al., 1996) aimed at the identification of genes possibly involved in LYP; however, mapping data suggested that it does not fall into the deletions described in patients affected by this disorder. However, it remained to be established whether it may be involved in the pathogenesis of other diseases mapped to Xq25, such as panhypopituitarism or Pettigrew syndrome

Additionally, NOV1 shares some identity with the Drosophila dumbfounded protein, which is a myoblast attractant that is essential for fusion. Aggregation and fusion of myoblasts to form myotubes is essential for myogenesis in many organisms. In Drosophila, the formation of syncytial myotubes is seeded by founder myoblasts. Founders fusion with clusters of fusion-competent myoblasts. The gene dumbfounded (duf) is required by myoblast aggregation and fusion. Duf encodes a member of the immunoglobulin superfamily of proteins that is an attractant for fusion-competent myoblasts. It is expressed by founder cells and serves to attract clusters of myoblasts from which myotubes form by fusion. See Ruiz-Gomez et al., Cell 102(2): 189-98 (2000).

NOV1b is potentially involved in tumorgenesis, including cell migration and invasion as well as metastatic potential. Therapeutic targeting of NOV1b with a monoclonal antibody is anticipated to limit or block the extent of tumor cell migration, invasion, and tumor metastasis, preferably in melanoma tumors.

The nucleic acids and proteins of NOV1 are useful in potential therapeutic applications implicated in various pathological disorders, described further below. For example, a cDNA encoding the NOV1 protein may be useful in gene therapy and may be useful when administered to a subject in need thereof.

The nucleic acids and proteins of the invention have applications in the diagnosis and/or treatment of various diseases and disorders. For example, the compositions of the present invention will have efficacy for the treatment of patients suffering from various tumors and cancers as well as other diseases, disorders and conditions.

The polypeptides can be used as immulnogens to produce antibodies specific for the invention, and as vaccines. They can also be used to screen for potential agonist and antagonist compounds. For example, a cDNA encoding the NOV1 protein may be useful in gene therapy, and the receptor-like protein may be useful when administered to a subject in need thereof. The novel NOV1 nucleic acid, and the NOV1 protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV1b protein has multiple hydrophilic regions, each of which can be used as an immunogen.

In one embodiment, a contemplated NOV1 a epitope is from about amino acids 10 to 110. In another embodiment, a NOV1 a epitope is from about amino acids 150 to 200. In additional embodiments, NOV1 a epitopes are from about amino acids 220 to 280, 290 to 325, 350 to 400, 420 to 430, 480 to 515, 550 to 560, 605 to 650, and from amino acids 660 to 841. Similarly, a contemplated NOV1b epitope is from about amino acid 25 to about amino acid 60. In additional embodiments, NOV1b epitopes are from about amino acids 65 to 110, 150 to 190, 240 to 275, and 280 to 334.

This novel protein also has value in development of powerful assay system for functional analysis of various human disorders, which will help in understanding of pathology of the disease and development of new drug targets for various disorders.

Expression data for NOV1 are included in Example 1.

NOV2

NOV2 is a novel LDL Receptor-like protein and nucleic acid encoding it.

The novel nucleic acid of 3264 nucleotides (21424344.9.6, SEQ ID NO:5) encoding a novel LDL Receptor-like protein is shown in Table 2A. An open reading frame (ORF) was identified beginning with an ATG initiation codon at nucleotides 544-546 and ending with a TGA codon at nucleotides 2683-2685. In Table 2A, the start and stop codons are in bold letters.

TABLE 2A
NOV2 Nucleotide Sequence

| | |
|---|---|
| CTGGGCGGGCGGGGGTACCGCCTGGTCAAGGGCCGGG | (SEQ ID NO:5) |
| GCGCCGGGCCGAGCCACCTCTTCTCGCGTCCCCCGCT | |
| TCCCTGTCGCGCTCCGCTGGCTGGACGCGCTGGAGGA | |
| GTGGAGCAGCACCCGGCCGGCCCTGGGGGCTGACAGT | |
| CGGCAAAGTTTGGCCCGAAGAGGAAGTGGTCTCAAAC | |
| CCCGGCAGGTGGCGACCAGGCCAGACCAGGGGCGCTC | |
| GCTGCCTGCGGGCGGGCTGTAGGCGAGGGCGCGCCCC | |
| AGTGCCGAGACCCGGGGCTTCAGGAGCCGGCCCCGGG | |
| AGAGAAGAGTGCGGCGGCGGACGGAGAAAACAACTCC | |
| AAAGTTGGCGAAAGGCACCGCCCCTACTCCCGGGCTG | |
| CCGCCGCCTCCCCGCCCCCAGCCCTGGCATCCAGAGT | |
| ACGGCACGAGCCCGGGCCATGGAGCCCCCCTGGGGAG | |
| GCGGCACCAGGGAGCCTGGGCGCCCGGGGCTCCGCCG | |
| CGACCCCATCGGGTAGACCACAGAAGCTCCGGGACCC | |
| TTCCGGCACCTCTGGACAGCCCAGGATGCTGTTGGCC | |
| ACCCTCCTCCTCCTCCTCCTTGGAGGCGCTCTGGCCC | |
| ATCCAGACCGGATTATTTTTCCAAATCATGCTTGTGA | |
| GGACCCCCCAGCAGTGCTCTTAGAAGTGCAGGGCACC | |
| TTACAGAGGCCCCTGGTCCGGGACAGCCGCACCTCCC | |
| CTGCCAACTGCACCTGGCTCATCCTGGGCAGCAAGGA | |
| ACAGACTGTCACCATCAGGTTCCAGAAGCTACACCTG | |
| GCCTGTGGCTCAGAGCGCTTAACCCTACGCTCCCCTC | |
| TCCAGCCACTGATCTCCCTGTGTGAGGCACCTCCCAG | |
| CCCTCTGCAGCTGCCCGGGGGAAACGTCACCATCACT | |
| TACAGCTATGCTGGGGCCAGAGCACCCATGGGCCAGG | |
| GCTTCCTGCTCTCCTACAGCCAAGATTGGCTGATGTG | |
| CCTGCAGGAAGAGTTTCAGTGCCTGAACCACCGCTGT | |
| GTATCTGCTGTCCAGCGCTGTGATGGGGTTGATGCCT | |
| GTGGCGATGGCTCTGATGAAGCAGGTTGCAGCTCAGA | |
| CCCCTTCCCTGGCCTGACCCCAAGACCCGTCCCCTCC | |
| CTGCCTTGCAATGTCACCTTGGAGGACTTCTATGGGG | |
| TCTTCTCCTCTCCTGGATATACACACCTAGCCTCAGT | |
| CTCCCACCCCCAGTCCTGCCATTGGCTGCTGGACCCC | |
| CATGATGGCCGGCGGCTGGCCGTGCGCTTCACAGCCC | |
| TGGACTTGGGCTTTGGAGATGCAGTGCATGTGTATGA | |
| CGGCCCTGGGCCCCCTGAGAGCTCCCGACTACTGCGT | |
| AGTCTCACCCACTTCAGCAATGGCAAGGCTGTCACTG | |
| TGGAGACACTGTCTGGCCAGGCTGTTGTGTCCTACCA | |
| CACAGTTGCTTGGAGCAATGGTCGTGGCTTCAATGCC | |
| ACCTACCATGTGCGGGGCTATTGCTTGCCTTGGGACA | |
| GACCCTGTGGCTTAGGCTCTGGCCTGGGAGCTGGCGA | |
| AGGCCTAGGTGAGCGCTGCTACAGTGAGGCACAGCGC | |
| TGTGACGGCTCATGGGACTGTGCTGACGGCACAGATG | |
| AGGAGGACTGCCCAGGCTGCCCACCTGGACACTTCCC | |
| CTGTGGGGCTGCTGGCACCTCTGGTGCCACAGCCTGC | |
| TACCTGCCTGCTGACCGCTGCAACTACCAGACTTTCT | |
| GTGCTGATGGAGCAGATGAGAGACGCTGTCGGCATTG | |
| CCAGCCTGGCAATTTCCGATGCCGGGACGAGAAGTGC | |
| GTGTATGAGACGTGGGTGTGCGATGGGCAGCCAGACT | |
| GTGCGGACGGCAGTGATGAGTGGGACTGCTCCTATGT | |
| TCTGCCCCGCAAGGTCATTACAGCTGCAGTCATTGGC | |
| AGCCTAGTGTGCGGCCTGCTCCTGGTCATCGCCCTGG | |
| GCTGCACCTGCAAGCTCTATGCCATTCGCACCCAGGA | |
| GTACAGCATCTTTGCCCCCCTCTCCCGGATGGAGGCT | |
| GAGATTGTGCAGCAGCAGGCACCCCCTTCCTACGGGC | |
| AGCTCATTGCCCAGGGTGCCATCCCACCTGTAGAAGA | |
| CTTTCCTACAGAGAATCCTAATGATAACTCAGTGCTG | |
| GGCAACCTGCGTTCTCTGCTACAGATCTTACGCCAGG | |
| ATATGACTCCAGGAGGTGGCCCAGGTGCCCGCCGTCG | |
| TCAGCGGGGCCGCTTGATGCGACGCCTGGTACGCCGT | |
| CTCCGCCGCTGGGGCTTGCTCCCTCGAACCAACACCC | |
| CGGCTCGGGCCTCTGAGGCCAGATCCCAGGTCACACC | |
| TTCTGCTGCTCCCCTTGAGGCCCTAGATGGTGGCACA | |
| GGTCCAGCCCGTGAGGGCGGCCAAGTGGGTGGGCAAG | |
| ATGGGGAGCAGGCACCCCCACTGCCCATCAAGGCTCC | |
| CCTCCCATCTGCTAGCACGTCTCCAGCCCCCACTACT | |
| GTCCCTGAAGCCCCAGGGCCACTGGCCTCACTGCCCC | |
| TAGAGCCATCACTATTGTCTGGAGTGGTGCAGGCCCT | |
| GCGAGGCCGCCTGTTGCCCAGCCTGGGGCCCCCAGGA | |
| CCAACCCGGAGCCCCCCTGGACCCCACACAGCAGTCC | |
| TGGCCCTGGAAGATGAGGACGATGTGCTACTGGTGCC | |
| ACTGGCTGAGCCGGGGGTGTGGGTAGCTGAGGCAGAG | |
| GATGAGCCACTGCTTACCTGAGGGGACCTGGGGGCTC | |
| TACTGAGGCCTCTCCCCTGGGGGCTCTACTCATAGTG | |
| GCACAACCTTTTAGAGGTGGGTCAGCCTCCCCTCCAC | |
| CACTTCCTTCCCTGTCCCTGGATTTCAGGGACTTGGT | |
| GGGCCTCCCGTTGACCCTATGTAGCTGCTATAAGTTA | |
| AGTGTCCCTCAGGCAGGGAGAGGGCTCACAGAGTCTC | |
| CTCTGTACGTGGCCATGGCCAGACACCCCAGTCCCTT | |
| CACCACCACCTGCTCCCCACGCCACCACCATTTGGGT | |
| GGCTGTTTTTAAAAAGTAAAGTTCTTAGAGGATCATA | |
| GGTCTGGACACTCCATCCTTGCCAAACCTCTACCCAA | |
| AAGTGGCCTTAAGCACCGGAATGCCAATTAACTAGAG | |
| ACCCTCCAGCCCCCAAGGGGAGGATTTGGGCAGAACC | |
| TGAGGTTTTGCCATCCACAATCCCTCCTACAGGGCCT | |
| GGCTCACAAAAAGAGTGCAACAAATGCTTCTATTCCA | |
| TAGCTACGGCATTGCTCAGTAAGTTGAGGTCAAAAAT | |
| AAAGGAATCATACATCTCAAAAAAAAAAAAAAAAAAA | |
| AAAAAAAA | |

The disclosed NOV2 polypeptide (SEQ ID NO:6) encoded by SEQ ID NO:5 is 713 amino acid residues and is presented using the one-letter code in Table 2B. The first 70 amino acids of the disclosed NOV2 protein were analyzed for signal peptide prediction and cellular localization. SignalP results predict that NOV2 is cleaved between position 16 and 17 of SEQ ID NO:6, i.e., at the slash in the amino acid sequence ALA-HP. Psort and Hydropathy profiles also predict that NOV2 contains a signal peptide and is likely to be localized at the plasma membrane (certainty of 0.4600).

TABLE 2B
Encoded NOV2 protein sequence.

| | |
|---|---|
| MLLATLLLLLLGGALA/HPDRIIFPNHACEDPPAVLL | (SEQ ID NO:6) |
| EVQGTLQRPLVRDSRTSPANCTWLILGSKEQTVTIRF | |
| QKLHLACGSERLTLRSPLQPLISLCEAPPSPLQLPGG | |
| NVTITYSYAGARAPMGQGFLLSYSQDWLMCLQEEFQC | |
| LNHRCVSAVQRCDGVDACGDGSDEAGCSSDPFPGLTP | |
| RPVPSLPCNVTLEDFYGVFSSPGYTHLASVSHPQSCH | |
| WLLDPHDGRRLAVRFTALDLGFGDAVHVYDGPGPPES | |
| SRLLRSLTHFSNGKAVTVETLSGQAVVSYHTVAWSNG | |
| RGFNATYHVRGYCLPWDRPCGLGSGLGAGEGLGERCY | |
| SEAQRCDGSWDCADGTDEEDCPGCPPGHFPCGAAGTS | |
| GATACYLPADRCNYQTFCADGADERRCRHCQPGNFRC | |
| RDEKCVYETWVCDGQPDCADGSDEWDCSYVLPRKVIT | |
| AAVIGSLVCGLLLVIALGCTCKLYAIRTQEYSIFAPL | |
| SRMEAEIVQQQAPPSYGQLIAQGAIPPVEDFPTENPN | |
| DNSVLGNLRSLLQILRQDMTPGGGPGARRRQRGRLMR | |
| RLVRRLRRWGLLPRTNTPARASEARSQVTPSAAPLEA | |
| LDGGTGPAREGGQVGGQDGEQAPPLPIKAPLPSASTS | |
| PAPTTVPEAPGPLASLPLEPSLLSGVVQALRGRLLPS | |
| LGPPGPTRSPPGPHTAVLALEDEDDVLLVPLAEPGVW | |
| VAEAEDEPLLT | |

NOV2 was originally cloned from pancreas and thyroid gland tissues, which were also used to express identifiable SeqCalling™ fragments of NOV2.

A search against the Patp database, a proprietary database that contains sequences published in patents and patent publications, yielded several homologous proteins. The full amino acid sequence of NOV2 was found to have 710 of 713 amino acid residues (99%) identical to, and 710 of 713 residues (99%) positive with the 713 amino acid residue human TANGO 136 protein, a LDL receptor-related protein, as seen in the PCT patent WO200026227-A1. SEQ ID NO:44 (E=0.0). Table 2C shows the alignment of these two proteins.

TABLE 2C
Alignment of NOV2 with TANGO 136 Protein (SEQ ID NO:44).

Score = 3949 (1354.9 bits), Expect 0.0, P = 0.0
Identities = 710/713 (99%), Positives 710/713 (99%), Frame = + 1

| | | | |
|---|---|---|---|
| Query: | 544 | MLLATLLLLLLGGALAHPDRIIFPNHACEDPPAVLLEVQGTLQRPLVRDSRTSPANCTWL | 723 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1 | MLLATLLLLLLGGALAHPDRIIFPNHACEDPPAVLLEVQGTLQRPLVRDSRTSPANCTWL | 60 |
| Query: | 724 | ILGSKEQTVTIRFQKLHLACGSERLTLRSPLQPLISLCEAPPSPLQLPGGNVTITYSYAG | 903 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 61 | ILGSKEQTVTIRFQKLHLACGSERLTLRSPLQPLISLCEAPPSPLQLPGGNVTITYSYAG | 120 |
| Query: | 904 | ARAPMGQGFLLSYSQDWLMCLQEEFQCLNHRCVSAVQRCDGVDACGDGSDEAGCSSDPFP | 1093 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 121 | ARAPMGQGFLLSYSQDWLMCLQEEFQCLNHRCVSAVQRCDGVDACGDGSDEAGCSSDPFP | 160 |
| Query: | 1084 | GLTPRPVPSLPCNVTLEDFYGVFSSPGYTHLASVSHPQSCHWLLDPHDGRRLAVRFTALD | 1263 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 181 | GLTPRPVPSLPCNVTLEDFYGVFSSPGYTHLASVSHPQSCHWLLDPHDGRRLAVRFTALD | 240 |
| Query: | 1264 | LGFGDAVHVYDGPGPPESSRLLRSLTHFSNGKAVTVETLSGQAVVSYHTVAWSNGRGFNA | 1443 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\| | |
| Sbjct: | 241 | LGFGDAVHVYDGPGPPESSRLLRSLTHFSNGKAVTVETLSGQAVVSYHTVAWGNGRGFNA | 300 |
| Query: | 1444 | TYHVRGYCLPWDRPCGLGSGLGAGEGLGERCYSEAQRCDGSWDCADGTDEEDCPGCPPGH | 1823 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 301 | TYHVRGYCLPWDRPCGLGSGLGAGEGLGERCYSEAQRCDGSWDCADGTDEEDCPGCPPGH | 360 |
| Query: | 1624 | FPCGAAGTSGATACYLPADRCNYQTFCADGADERRCRHCQPGNFRCRDEKCVYETWVCDG | 1803 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 361 | FPCGAAGTSGATACYLPADRCNYQTFCADGADERRCRHCQPGNFRCRDEKCVYETWVCDG | 420 |
| Query: | 1804 | QPDCADGSDEWDCSYVLPRKVITAAVIGSLVCGLLLVIALGCTCKLYAIRTOEYSIFAPL | 1983 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SbjCt: | 421 | QPDCADGSDEWDCSYVLPRKVITAAVIGSLVCGLLLVIALGCTCKLYAIRTQEYSIFAPL | 480 |
| Query: | 1984 | SRMEAEIVQQQAPPSYGQLIAQGAIPPVEDFPTENPNDNSVLGNLRSLLQILRQDMTPGG | 2163 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 481 | SRMEAEIVQQQAPPSYGQLIAQGAIPPVEDFPTENPNDNSVLGNLRSLLQILRQDMTPGG | 540 |
| Query: | 2164 | GPGARRRQRGRLMRRLVRRLRRWGLLPRTNTPARSAEARSQVTPSAAPLEALDGGTGPAR | 2163 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 541 | GPGARRRQRGRLMRRLVRRLRRWGLLPRTNTPARSAEARSQVTPSAAPLEALDGGTGPAR | 600 |
| Query: | 2344 | EGGQVGGQDGEQAPPLPIKAPLPSASTSPAPTTVPEAPGPLASLPLEPSLLSGVVQALRG | 2523 |
| | | \|\|\| \|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\| | |
| Sbjct: | 601 | EGGQVGGQDGEQAPPLPIKAPLPSASTSPAPTTVPEAPGPLASLPLEPSLLSGVVQALRG 660 | |
| Query: | 2524 | RLLPSLGPPGPTRSPPGPHTAVLALEDEDDVLLVPLAEPGVWVAEAEDEPLLT 2682 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | | RLLPSLGPPGPTRSPPGPHTAVLALEDEDDVLLVPLAEPGVWVAEAEDEPLLT 713 | |

In addition, the NOV2 protein also shows extensive homology to murine TANGO 136 partial protein, as seen in the PCT patent WO200026227-A1. The NOV2 protein also has extensive homology to the Human Receptor Protein (HURP) 7, as seen in the PCT patent WO9941375-A2. In addition, NOV2 has good homology to the human Breast and Ovarian Cancer Associated Antigen Protein, as seen in the PCT patent WO200055173-A1. Finally, NOV2 is similar to three hypothetical human proteins: PRO724, PRO724 (UNQ389) and ORFX ORF2010, as seen in WO9946281-A2, WO200053756-A2 and WO200058473, respectively.

TABLE 2D
Patp alignments of NOV2

| | Reading | High | |
|---|---|---|---|
| Sequences producing High-scoring Segment Pairs: | Frame | Score | Expect |
| patp:AAY41712 Human PRO724 Protein - H. sapiens | +1 | 3853 | 0.0 |
| patp:AAB44268 Human PRO724 (UNQ309)Protein - H. sapiens | +1 | 3853 | 0.0 |
| patp:AAY71081 Human TANGO 136 Procein - H. sapiens . . . | +1 | 3849 | 0.0 |
| patp:AAY15228 Human Receptor Protein 7 Amino Acid Sequ | +1 | 3053 | 0.0 |
| patp:AAY71080 Murine TANGO Partial Protein - Mus sp . . . | +1 | 2846 | 1.3e-295 |
| patp:AAB59032 Breast and Ovarian Cancer Associated . . . | +1 | 2590 | 1.7e-268 |
| patp:AAB42248 Human ORFX ORF2010 Polypeotide Squence . . . | +1 | 1531 | 1.3e-161 |

A BLAST search against public databases revealed that the disclosed NOV2 protein (SEQ ID NO:6) has significant homology with a family of LDL Receptor Related Proteins and a potential tumor suppressor, as shown in Table 2E. NOV2 was also found to have 246 of 510 residues (48%) identical to, and 297 of 510 (58%) positive with human LDL Receptor Related Protein 105 (ACC:075074, E value=9.3e-123). In addition, NOV2 was found to have 293 of 296 residues (98%) identical to, and 293 of 296 residues (98%) positive with, a hypothetical protein DKFZp564C1940.1 from Homo sapiens (PIR-ID:T12469, E value=7.5e-162).

TABLE 2E
BLAST results for NOV2

| | | | | Pos- | |
|---|---|---|---|---|---|
| Gene Index/ | Protein/ | Length | Identity | itives | |
| Identifier | Organism | (aa) | (%) | (%) | Expect |
| Gi\|12667806\|ref\|N | Low-density | 713 | 493/695 | 507/695 | 0.0 |
| P_075369.1 | lipoprotein | | (70%) | (72%) | |
| | receptor- | | | | |
| | related | | | | |
| | protein | | | | |
| | 10 | | | | |
| | Mus | | | | |
| | musculus | | | | |
| Gi\|11425836\|ref\|X | Low-density | 770 | 228/552 | 289/552 | 4e−94 |
| P_009183.1\| | lipoprotein | | (41%) | (52%) | |
| | receptor- | | | | |
| | related | | | | |
| | protein | | | | |
| | 3 | | | | |
| | Homo | | | | |
| | sapiens | | | | |
| Gi\|4505015\|ref\|NP | Low-density | 770 | 228/552 | 289/552 | 5e−94 |
| 002324.1\| | lipoprotein | | (41%) | (52%) | |
| | receptor- | | | | |
| | related | | | | |
| | protein | | | | |
| | 3 | | | | |
| | Homo | | | | |
| | sapiens | | | | |
| Gi\|7513998\|pir\|T0 | LDL | 770 | 228/552 | 289/552 | 1e−93 |
| 0203 | receptor- | | (41%) | (52%) | |
| | related | | | | |
| | protein | | | | |
| | 105 | | | | |
| | rat | | | | |
| Gi\|7305525\|ref\|NP | Potential | 859 | 207/554 | 282/554 | 4e−88 |
| _038465.1\| | tumor | | (37%) | (50%) | |
| | suppressor | | | | |
| | Homo | | | | |
| | sapiens | | | | |

This information is presented graphically in the multiple sequence alignment given in Table 2F (with NOV2 being shown on line 1) as a ClustalW analysis comparing NOV2 with related protein sequences.

[image]

[image]

[image]

DOMAIN results for NOV2 were collected from the Conserved Domain Database (CDD) with Reverse Position Specific BLAST. This BLAST samples domains found in the Smart and Pfam collections. The NOV2 protein showed significant alignment with Smart00192 (LDLa, Low-density lipoprotein receptor domain class A, E=4e-09), Pfam 00057 (ldl_recept_a, Low-density lipoprotein receptor domain class A, E=4e-9), Smart 00042 (CUB, CUB domain, E=5e-08) and Pfam00431 (CUB, CUB domain, E=5e-06). Table 2G shows the results of domain analysis.

TABLE 2G
Domain analysis for NOV2

| Gene index identifier/Domain Name | Results |
|---|---|
| Gnl\|Smart\|smart00192, LDLa, Low density | CD-Length = 38 residues, 97.4% aligned |
| lipoprotein receptor family domain class A | Score = 56.6 bits (135), Expect = 4e−09 |
| Gnl\|Smart\|smart00192, LDLa, Low density | CD-Length = 38 residues, 97.4% aligned |
| lipoprotein receptor family domain class A | Score = 53.9 bits (128), Expect = 3e−08 |
| Gnl\|Pfam\|pfam00057, ldl_recept_a, Low | CD-Length = 39 residues, 92.3% aligned |
| density lipoprotein receptor family domain | Score = 56.6 bits (135), Expect = 4e−09 |
| class A | |
| Gnl\|Pfam\|pfam00057, ldl_recept_a, Low | CD-Length = 39 residues, 94.9% aligned |
| density lipoprotein receptor family domain | Score = 47.8 bits (112), Expect = 2e−06 |
| class A | |
| Gnl\|Smart\|smart00042, CUB | CD-Length = 114 residues, 97.4% aligned |
| | Score = 53.1 bits (126), Expect = 5e−08 |
| Gnl\|Smart\|smart00042, CUB | CD-Length = 114 residues, 97.4% aligned |
| | Score = 39.3 bits (90), Expect = 7e−04 |
| Gnl\|Pfam\|pfam0043l, CUB, CUB domain | CD-Length = 110 residues, 100.0% aligned |
| | Score = 46.6 bits (109), Expect = 5e−06 |

NOV2 has two LDL receptor family domain class A-like domains. The LDL receptor family domain class A is a Cysteine-rich repeat in the LDL receptor that plays a central role in mammalian cholesterol mechanism. Repeats of this domain are thought to be involved in ligand binding (Yamamoto et al. (1984) Cell 39:27-38; and Fass et al. (1997)Nature 388:691-693). Table 2H and Table 2I show the alignment of each of the two LDL receptor class A domains of NOV2 with a LDL receptor class A domain consensus sequence (SEQ ID NO:50).

[image]

NOV2 has two CUB-like domains (amino acids 34 to 88 and amino acids 192 to 246). The CUB domain is an extracellular domain of approximately 110 residues which is found in functionally diverse, mostly developmentally-regulated proteins. (See PROSITE: PDOC00908) For example, Spermadhesins contain only this domain. Amino acids 4 through 63 of NOV2 align with amino acids 192 through 246 of the 1 14 residue CUB domain. Table 2J and Table 2K depict the alignment of the each of the two CUB-like domain of NOV2 with a CUB domain consensus sequence (SEQ ID NO:53).

[image]

[image]

The similarities to the low-density lipoprotein receptor family domain class A and the CUB domain indicate that the NOV2 sequence has properties similar to those of other proteins known to contain these domains.

NOV2 has extensive homology with multiple LDL receptor related proteins from different organisms, including the human LDL receptor-related protein 3 (LRP-3), the mouse LDL receptor-related protein 10, the rat LDL receptor-related protein, and human TANGO 136 protein. Accordingly, NOV2 is a novel member of the LDL receptor family, which includes LDLR, LRP-2, LRP-3, LRP-5, LRP-6, and LR8B. Members of this family are endocytic receptors that bind and internalize ligands from the circulation and extracellular space. Thus, NOV2 has utility in that it functions similarly to other members of the low density lipoprotein receptor family.

LDL receptors binds plasma lipoproteins that contain apolipoprotein B-100 (apoB-100) or apoE on their surface. LDL receptor is critical for the uptake of these lipoproteins, and mutations in LDL receptor are the cause of familial hypercholesterolemia, a disorder characterized by high levels of cholesterol-rich LDL in the plasma. The elevation of plasma cholesterol levels in patients afflicted with familial hypercholesterolemia leads to atherosclerosis and increased risk for myocardial infarction. NOV2 potentially plays a role in disorders of lipoprotein metabolism and transport, e.g., cardiovascular diseases such as atherosclerosis. Accordingly, NOV2 nucleic acids, proteins and NOV2 antagonists and agonists are useful for treatment of disorders of lipoprotein metabolism and transport, e.g., cardiovascular diseases such as atherosclerosis. For example, a cDNA encoding the NOV2 protein may be useful in gene therapy, and the NOV2 protein may be useful when administrated to a subject in need thereof.

In vitro studies have shown that LRP-2 is capable of binding and mediating the cellular uptake of a large number of different ligands including apoE-enriched very low density lipoproteins (Willnow et al. (1992) J. Biol. Chem. 267:26172-26180), complexes of urokinase plasminogen activator and plasminogen activator inhibitor-1 (tPA:PAI-1) (Willnow et al., supra), lipoprotein lipase (Willnow et al., supra), and lactoferrin. A receptor associated protein known as RAP (Orlando et al. (1992) Proc. Natl. Acad. Sci. 89:6698-6702) inhibits the binding of these ligands to LRP-2. Some or all of these ligands may bind NOV2. Accordingly, NOV2 nucleic acids, polypeptides, antagonists and agonists are useful for treatment of clotting disorders, e.g., inhibiting clot formation or dissolving clots.

A few specific and physiologically relevant ligands for LRP-2 have been identified, including apolipoprotein J (apoJ)/clusterin (Kounnas et al. (1995) J. Biol. Chem. 22: 13070-13075) and thyroglobulin (Zheng et al. (1998) Endocrinology 139:1462-1465). ApoJ has been reported to bind several proteins, including the βA4 peptide of the Alzheimer's precursor protein, a subclass of high density lipoprotein, and the complement membrane attack complex C5-C9 (Kounnas et al., supra). The clearance of apoJcomplexed with these and other molecules is expected to occur via LRP-2. Thus, LRP-2 may play an important functional role in the clearance of these complexes. For example, LRP-2 may function to target lipoproteins for clearance or may inhibit the cytolytic activity of the complement membrane C5b-C9 by clearing the apoJ/C5b-C9 complex. The fact that LRP-2 can bind the apoJ/amyloid-B complex suggests that LRP-2 may be involved in regulating the pathogenesis of Alzheimer's disease. A role for LRP-2 in Alzheimer's disease is further supported by another study that showed that LRP-2 may be involved in transporting the apoJ/amyloid 13 complex across the blood-brain-barrier (Zlokovic et al. (1996) Proc. Natl. Acad. Sci. 93:422904234). Thus, NOV2 nucleic acids, proteins, agonists and antagonists are useful for the treatment of Alzheimer's disease and other neurodegenerative disorders, e.g., Huntington's disease and Parkinson's disease.

LRP-2 is involved in participating in the endocytosis of thyroglobulin, which results in the release of thyroid hormones (Zheng et al. (1998) Endocrinology 139:1462-65). NOV2 may also be involved in the regulating the release of thyroid hormones. Thus, NOV2 nucleic acids, proteins, agonists, and antagonists are useful for the treatment of thyroid disorders, e.g., thyroid hormone release disorders.

LRP-2 is also predicted to play a role as a drug receptor and is thought to be involved in the uptake of polybasic drugs, e.g., aprotinin, aminoglycosides and polymyxin B. The uptake of polybasic drugs can be toxic, e.g., the administration of aminoglycosides is often associated with nephro- and ototoxicity. NOV2 may also mediate uptake of polybasic drugs, and NOV2 nucleic acids, proteins, agonists and antagonists are useful for the modulating the uptake of such drugs. NOV2 can also be used to design less toxic versions of such drugs.

In addition, LRP-2 is involved in the pathogenesis of Heymann Nephritis nephropathy (HN), an autoimmune glomerular disease, which is similar to human membranous nephropathy. It is thought that LRP-2 is the major pathogenic antigen and forms an antigen-antibody complex between the glomerular basement membrane and the foot processes of glomerular epithelial cells. The presence of the antigen-antibody complex leads to extensive damage of the basement membrane and proteinuria (Farquhar et al. (1994) Ann. N.Y Acad. Sci. 97-106). Similar to LRP-2, NOV2 may play a pathogenic role in autoimmune glomerular disease. Thus, NOV2 nucleic acids, proteins, agonists and antagonists are useful for the treatment of autoimmune glomerular disease.

LRP-5 and LRP-6 are thought to function in endocytosis. Based on genetic evidence, LRP-5 and possibly LRP-6 are thought to play a role in the molecular pathogenesis of type I diabetes (Brown et al. (1998) Biochem. Biophys. Res. Comm. 248:879-888). NOV2 is also likely to play a role in type I diabetes. Thus, NOV2 nucleic acids, proteins, agonists and antagonists are useful for the treatment of type I diabetes.

LR8B is expressed in brain and might be involved in brain-specific lipid transport. Brain-specific lipid transport may involve apoE4, which is associated with Alzheimer's disease. NOV2 may also be involved in brain-specific lipid transport, and NOV2 nucleic acids, proteins, agonists and antagonists are useful for the treatment of Alzheimer's disease.

In general, the NOV2 compositions of the present invention will have efficacy for treatment of neurological disorders, e.g., neurodegenerative disorders and neuropsychiatric disorders. Examples of neurodegenerative disorders include Alzheimer's disease, Parkinson's disease, and Huntington's disease. Examples of neuropsychiatric disorders include schizophrenia, attention deficit disorder, unipolar affective (mood) disorder, bipolar affective (mood) disorders (e.g., severe bipolar affective disorder (BP-I) and bipolar affective disorder with hypomania and major depression (BP-II)), and schizoaffective disorders. Other LDL-receptor related diseases and disorders are contemplated.

In addition to the homology to the LDL receptor-related proteins, the NOV2 protein also has extensive homology to the Breast and Ovarian Cancer Associated Antigen protein (from the Patp result), and to a potential human tumor suppressor protein (from the Blast result). Accordingly, the NOV2 compositions of the present invention will have efficacy for treatment of cancer, particularly breast and ovarian cancer.

The novel nucleic acid encoding NOV2, and the NOV2 protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods and other diseases, disorders and conditions of the like. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below.

For example, the disclosed NOV2 protein has multiple hydrophilic regions, each of which can be used as an immunogen. In one embodiment, a contemplated NOV2 epitope is from about amino acids 310 to 360. In another embodiment, a NOV2 epitope is from about amino acids 380 to 430. In additional embodiments, a NOV2 epitope is from about amino acids 520 to 600. In a further embodiment, a NOV2 epitope is from about amino acids 600 to 625. These novel proteins can also be used to develop assay systems for functional analysis.

NOV3

NOV3 is a novel small inducible cytokine family protein and nucleic acid encoding it.

The novel nucleic acid of 1265 nucleotides (B80173.9.32, SEQ ID NO:7) encoding a small inducible cytokine-like protein is shown in Table 3A. An open reading frame (ORF) was identified beginning with an ATG initiation codon at nucleotides 61-63 and ending with a TGA codon at nucleotides 544-546. In Table 3A, the start and stop codons are in bold letters.

TABLE 3A
NOV3 Nucleotide Sequence (SEQ ID NO: 7)

GCACCTCCTCGCCAGCTCTTCCGCTCCTCTCACAGCCGCCAGACCCGCCTGCTGAGCCCCATGGCCCGCG
CTGCTCTCTCCGCCGCCCCCAGCAATCCCCGGCTCCTGCGAGTGGCACTGCTGCTCCTGCTCCTGGTAGC
CGCTGGCCGGCGCGCAGCAGGAGCGTCCGTGGCCACTGAACTGCGCTGCCAGTGCTTGCAGACCCTGCAG
GGAATTCACCCCAAGAACATCCAAAGTGTGAACGTGAAGTCCCCCGGACCCCAATGCGCTCAAACCGAAG
TCGACTTCGGTTTGAGCGCATTGGCTACCCCGGATATGACGTGGCGTATGTACTCGTGCCATAACCAAAA
TCTTCATAATATTCTCTTTCTGTCACAAATTTTTGGTAGTTTTTCAGGTTTTGCATCCATGACATCGGGA
TCCCACGACCCAATGCGCTCAAACCGAAGTCCACTCAAGAATGGGCGGAAAGCTTGCCTCAATCCTGCAT
CCCCCATAGTTAAGAAAATCATCGAAAAGATGCTGAACAGTGACAAATCCAACTGACCAGAAGGGAGGAG
GAAGCTCACTGGTGGCTGTTCCTGAAGGAGGCCCTGCCCTTATAGGAACAGAAGAGGAAAGAGAGACACA
GCTGCAGAGGCCACCTGGATTGTGCCTAATGTGTTTGAGCATCGCTTAGGAGAAGTCTTCTATTTATTTA
TTTATTCATTAGTTTTGAAGATTCTATGTTAATATTTTAGGTGTAAAATAATTAAGGGTATGATTAACTC
TACCTGCACACTGTCCTATTATATTCATTCTTTTTGAAATGTCAACCCCAAGTTAGTTCAATCTGGATTC
ATATTTAATTTGAAGGTAGAATGTTTTCAAATGTTCTCCAGTCATTATGTTAATATTTCTGAGGAGCCTG
CAACATGCCAGCCACTGTGATAGAGGCTGGCGGATCCAAGCAAATGGCCAATGAGATCATTGTGAAGGCA
GGGGAATGTATGTGCACATCTGTTTTGTAACTGTTTAGATGAATGTCAGTTGTTATTTATTGAAATGATT
TCACAGTGTGTGGTCAACATTTCTCATGTTGAAACTTTAAGAACTAAAATGTTCTAAATATCCCTTGGAC
ATTTTATGTCTTTCTTGTAAGGCATACTGCCTTGTTTAATGGTAGTTTTACAGTGTTTCTGGCTTAGAAC
AAAGGGGCTTAATTATTGATGTTTTCATAGAGAATATAAAAATAAAGCACTTATAGAAAAAAAAAAAAAA
AAAAA

The disclosed NOV3 polypeptide (SEQ ID NO:8) encoded by SEQ ID NO:7 is 161 amino acid residues and is presented using the one-letter code in Table 3B. The first 70 amino acids of the disclosed NOV3 protein were analyzed for signal peptide prediction and cellular localization. SignalP results predict that NOV3 is cleaved between position 34 and 35 of SEQ ID NO:8, i.e., at the slash in the amino acid sequence AAG-AS. Psort and Hydropathy profiles also predict that NOV3 contains a signal peptide and is likely to be localized at the mitochondrial inner membrane (certainty of 0.7182). Based on Hydropathy plot, NOV3 residues from about 8 to about 40, and from about 95 to about 118 are predicted to be transmembrane domains. Residues from about 45 to about 75, from about 83 to about 98 and from about 118 to about 148 are predicted to contain three hydrophilic regions.

TABLE 3B
Encoded NOV3 protein sequence (SEQ ID NO: 8).

MARAALSAAPSNPRLLRVALLLLLLVAAGRRAAG/ASVATELRCQCLQTLQGIHPKNIQSVNVKSPGPQCA
QTEVDFGLSALATPDMTWRMYSCHNQNLHNILFLSQIFGSFSGFASMTSGSHDPMRSNRSPLKNGRKACLN
PASPIVKKIIEKMLNSDKSN

Human tissues express identifiable SeqCalling™ fragments of NOV3 include NHFLS, HCN and HFLSRA.

A BLAST search was performed against public protein databases. The full amino acid sequence of the protein of the invention was found to have extensive homology with GRO family proteins including GRO1/Gro.alpha, GRO2/Gro.beta and GRO3/Gro.gamma. The GRO proteins belong to a super-family of related small inducible cytokines. (OMIM 155730, 139110 and 139111) For example, NOV3 has 79 of 161 residues (49%) identical to, and 82/161 residues (50%) positive with, the 107 amino acid residue human GROI protein (Gi|4504153|ref|NP_{—}001502.1) (E value=2e-23). GRO1 is also known as Gro.alpha, Melanoma Growth Stimulatory Activity (MGSA) and Neutrophil-Activation Protein 3 (NAP-3). The results of the BLAST search are summarized in Table 3C.

TABLE 3C
BLAST results for NOV3

| | | | | Pos- | |
|---|---|---|---|---|---|
| Gene Index/ | Protein/ | Length | Identity | itives | |
| Identifier | Organism | (aa) | (%) | (%) | Expect |
| Gi\|4504153\|ref\| | GRO1 | 107 | 79/161 | 82/161 | 2e−23 |
| NP_001502.1\| | Oncogene | | (49%) | (50%) | |
| | (melanoma | | | | |
| | growth | | | | |
| | stimulating | | | | |
| | activity, | | | | |
| | alpha) | | | | |
| | Homo | | | | |
| | sapiens | | | | |
| Gi\|640276\|pdb\|1 | Human | 73 | 66/126 | 69/126 | 6e−20 |
| MGS\|A | Melanoma | | (52%) | (54%) | |
| | Growth | | | | |
| | Stimulating | | | | |
| | Activity | | | | |
| | (MgsaGRO_{—} | | | | |
| | ALPHA) | | | | |
| | Homo | | | | |
| | sapiens | | | | |
| Gi\|999730\|pdb\|1 | Human | 72 | 65/125 | 68/125 | 2e−19 |
| MS\|A | Melanoma | | (52%) | (54%) | |
| | Growth | | | | |
| | Stimulating | | | | |
| | Activity | | | | |
| | (MgsaGRO_{—} | | | | |
| | ALPHA) | | | | |
| | Mutation | | | | |
| | with the | | | | |
| | last Asn | | | | |
| | truncated | | | | |
| Gi\|4504155\|ref\| | GRO2 | 107 | 69/161 | 76/161 | 2e−18 |
| NP_002080.1\| | Oncogene | | (42%) | (46%) | |
| | Homo | | | | |
| | sapiens | | | | |
| Gi\|13632683\|ref | GN03 | 107 | 66/161 | 75/161 | le−17 |
| \|XP_003508.2\| | Oncongene | | (40%) | (45%) | |
| | Homo | | | | |
| | sapiens | | | | |

This information is presented graphically in the multiple sequence alignment given in Table 3D (with NOV3 being shown on line 1) as a ClustalW analysis comparing NOV3 with related protein sequences. Interestingly, the ClustalW alignment reveals that the homology between NOV3 and related proteins are more extensive than the homology figures (around 50% for various proteins) listed in the BLAST results. The N-terminal stretch of 74 amino acids and a C-terminal stretch of 31 amino acids of the NOV3 protein have high level of homology with other related proteins, as indicated graphically by two almost solid identity blocks extending between NOV3 and other related proteins. For example, over the N-terminal stretch, NOV3 has 73 of 74 residues (99%) identical to the human GRO1 Oncoprotein, 68 of 74 (92%) identical to the human GRO2 protein, and 68 of 74 (92%) identical to the human GR03 protein. Over the C-terminal stretch, NOV3 has 29 of 30 residues (97%) identical to the human GRO1 Oncoprotein, 24 of 30 (80%) identical to the human GR02 protein, 22 of 30 residues (73%) identical to the human GRO3 protein.

[image]

[image]

DOMAIN results for NOV3 were collected from the Conserved Domain Database (CDD) with Reverse Position Specific BLAST. This BLAST samples domains found in the Smart and Pfam collections. The NOV3 protein aligned with a number of related domains in both collections. Table 3E summarizes the results of domain search.

TABLE 3E
Domain analysis for NOV3

| Gene index | |
|---|---|
| identifier/Domain Name | Results |
| Gn1\|Pfam\|pfam00048, IL8, | CD-Length = 67 residues, 83.6% |
| Small cytokines, inerleukin-8 | aligned Score = 37.7 bits (86), |
| like | Expect = 5e-04 |
| Gn1\|Pfam\|pfam00048, IL8, | CD-Length = 67 residues, only 34.3% |
| Small cytokines, inerleukin-8 | aligned |
| like | Score = 34.7 bits (78), Expect = 0.004 |
| Gn1\|Smart\|smart00199, SCY, | CD-Length = 60 residues, only 38.3% |
| Intercrine alpha family | aligned Score = 33.9 bits (76), Expect = |
| (chemokine CXC) | 0.007 |

NOV3 has two IL8-like domains (amino acids 36 to 88 and amino acids 132 to 154). Table 3F depicts the alignment of the cL8-like domains of NOV3 with a IL8 consensus sequence (SEQ ID NO:61).

NOV3 also has a SCY-like domain (amino acids 132-154). The SCY domain is found in intercrine alpha family, a family of cytokines involved in cell-specific chemotaxis, mediation of cell growth, and the inflammatory response. Table 3G depicts the alignment of the SCY-like domain of NOV2 with a SCY domain consensus sequence (SEQ ID NO:62).

[image]

As the BLAST result indicates, NOV3 protein shows good homology with GRO proteins, including GRO1/Gro.alpha, GRO2/Gro.beta and GRO3/Gro/.Gamma. The GRO genes belong to a gene super-family which encodes a set of related small inducible cytokines that includes NAP-1/IL-8 (hereinafter IL-8/GRO gene family) (Matsushima, K., et al., 1988, J. Exp. Med., 167:1883-1893; Schmid, J., et al., 1987, J. of Immunol., 139:250-256; Peveru, P., et al., 1988, J. Exp. Med., 167:1547-1559), and platelet basic protein (PBP). PBP is the precursor of connective tissue activating protein III (CTAP IlI), .beta.-thromboglobulin (Castor, C. W., et al., 1983, Proc. Nat. Acad. Sci., 80:76-769), platelet factor 4 (PF4) (Deuel, T. F, et al. 1977, Proc. Nat. Acad. Sci. 74:2256-2258), .gamma.-interferon-inducible peptide (.gamma.IP-10) (Luster, A. D., et al., 1985, Nature (London), 315:672-676), and macrophage inflammatory protein 2 (MIP-2) (Wolpe, S. D., et al., 1989, PNAS (USA), 86:612-616).

GRO1 was initially identified by its constitutive over-expression in spontaneously transformed Chinese hamster fibroblasts (Anisowicz, A., et al., 1987, PNAS , 84:7188-7192). A related gene was identified in v-src transformed chicken cells (Sugano, S., et al., 1987, Cell, 49:321-328; Bedard, P. A., et al., 1987, PNAS (USA), 84:6715-6719). In expression studies with normal fibroblasts, Gro showed early response kinetics similar to c-fos, leading to the name Gro (growth regulated) (Anisowicz, et al., 1987, supra). Later, a protein with melanoma stimulating activity (MGSA) (Richmond, A., et al., supra) was shown to be encoded by GRO1, and sequence similarity was reported with the murine early response gene KC (Oquendo, P., et al., 1989, J. Biol. Chem., 264:4133-4137).

Preliminary studies showed that the Gro ax gene was expressed in active ulcerative colitis disease, but not in the inactive tissue. (Isaacs, K., et al., “Profiles of cytokine activation in inflammatory bowel disease tissue: measurement of cDNA amplification”, American Gastroenterological Assoc. & American Assoc. for the Study of Liver Diseases, May 13-16, 1990, Texas (Abstract)). On the other hand, disparity in expression of the Gro .alpha. gene was less in the case of active versus inactive tissues from Crohn's disease. The expression of the Gro .alpha. gene in active intestinal inflammation suggests a role of these cytokines in the pathogenesis of inflammatory bowel disease. In addition, GRO genes exhibit differential expression patterns in various tumor cells compared to their normal counterparts. For example, Gro.gamma. was found in colonic epithelial tumor cells but not in adjacent normal epithelial cells. It has also been observed that Gro alpha. is over-expressed in other tumor cell lines such as CHEF/16 cells, src-transformed chicken fibroblasts, and human melanomas. See U.S. Pat. No. 5,994,060 Example 1. On the other hand, it has also been observed that Gro alpha. is expressed in normal growing mammary cells but was absent in many carcinomas (Anisowicz, A. et al., 1988, Proc. Nat. Acad. Sci. supra.). Thus, depending on the cell and tumor types in question, a tumor treatment regimen would involve varying the amount of Gro.beta. or .gamma. accessible to the cells. This can be achieved by either increasing or decreasing the amounts of the GRO proteins available to the cells, depending on whether the tumorigenesis is due to their over- or under- expression of the GRO genes.

The similarity information for the NOV3 protein and nucleic acid disclosed herein suggest that NOV3 is a novel member of the IL8/GRO protein family. The result of domain analysis that NOV3 contains two IL-8-like domains further demonstrates that NOV3 is a novel member of the IL8/GRO protein family. Consequently, NOV3 nucleic acids, proteins, agonists and antagonists may have potential diagnostic and therapeutic utilities in various diseases and disorders that involve IL-8/Gro family genes and/or other related pathologies, including but not limited to Crohn's disease, inflammatory bowel disease, ulcerative colitis and various types of cancers, specifically colon cancer. For example, a cDNA encoding the NOV3 protein may be useful in gene therapy, and the NOV3 protein may be useful when administered to a subject in need thereof.

In addition, several members of the IL8/GRO family of proteins were shown to have neutrophil activating functions. IL-8 was the first one to be identified to have potent neutrophil activating function. Walz, A. et al., Biochem. Biophys. Res. Commun. 149:755 (1987), Schroder, J. M. et al., Immunol. 139:3474 (1987), Yoshimura, T. et al., Proc. Natl. Acad. Sci. U.S.A. 84:9233 (1987) and Baggiolini, M. et al., J. Clin. Invest. 84:1045 (1989). Subsequently, two other proteins of this family, neutrophil-activating peptide 2 (NAP-2) and Gro.alpha. were demonstrated to have similar biological activities on human neutrophils. Walz, A. and M. Baggiolini, Biochem. Biophys. Res. Commun. 159:969 (1989), Walz, A. and M. Baggiolini, J. Exp. Med. 171:449 (1990), Richmond, A. et al., EMBO J. 7:2025 (1988), Moser, B. et al., J. Exp. Med. 171:1797 (1990) and Schroder, J.-M. et al., J. Exp. Med. 171:1091 (1990).

Because of the extensive sequence similarity of NOV3 with GRO proteins, NOV3 is also likely to have the function of neutrophil activation. Thus, the nucleic acids and proteins of NOV3 are useful in therapeutic applications implicated in various neutrophil deficient pathological disorders in mammals, preferably humans. More specifically, NOV3, like other neutrophil-activating proteins, will be useful in the treatment of conditions which are accompanied or caused, locally or systemically, by a modification of the number or activation state of the PMN (polymorphonuclear cells—neutrophils). An increase of the number or enhancement of the activation state of the PMN leads to clinical improvement in bacterial, mycoplasma, yeast, fungal, and in various vital infections.

By way of nonlimiting example, NOV3 nucleic acids and polypeptides will have efficacy for treatment of patients suffering from various disorders associated with abnormally high or low neutrophil count and/or generalized high/low neutrophil level, including, for example, inflammatory illnesses, hematopoietic deficits arising from chemotherapy or from radiation therapy and resulting disorders derived from the above conditions. NOV3 nucleic acids and polypeptides will also be useful in enhancing the success of bone marrow transplants and wound healing burn treatment. Other diseases and disorders involving neutrophil activation and disorders are contemplated.

In general, the NOV3 nucleic acids and protein are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon. The polypeptides can also be used as immunogens to produce antibodies specific for the invention, and as vaccines. They can also be used to screen for potential agonist and antagonist compounds.

The novel nucleic acid encoding the NOV3 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example, as mentioned above, the disclosed NOV3 protein has multiple hydrophilic regions, each of which can be used as an immunogen. The novel NOV3 protein can be used in assay systems for functional analysis of various human disorders, which will help in understanding of pathology of the disease and development of new drug targets for various disorders.

NOV4

A NOV4 sequence (83614984.0.5, SEQ ID NO:9) according to the invention includes a nucleic acid sequence of 642 nucleotides encoding a cell cycle and proliferation protein-related protein (“CCYPR”). Table 4A shows the nucleotide sequence of NOV4. An open reading frame was identified beginning with an ATG initiation codon at nucleotides 207-209 and ending with a TAA stop codon at nucleotides 539-541. The start and the stop codons are in bold letters. In another embodiment, NOV4 nucleic acid sequence contains a deletion of T at nucleotide 594, and an additional sequence of AAAAAAAAAAAAAAGC (SEQ ID NO:64) at the 3′ end.

TABLE 4A
NOV4 Nucleotide Sequence (SEQ ID NO:9).

CTTCAGTGTGCATGTTCCTTGGACACCTGCCTCAGTGTGCATGTTCACTGGGCATCTTCCCTTCGACCCCT
TTGCCCACGTGGTGACCGCTGGGGAGCTGTGAGAGTGTGAGGGGCACGTTCCAGCCGTCTGGACTCTTTCT
CTCCTACTGAGACGCAGCCTATAGGTCCGCAGGCCAGTCCTCCCAGGAACTGAAATAGTGAAATATGAGTT
GGCGAGGAAGATCAACATATAGGCCTAGGCCAAGAAGAAGTTTACAGCCTCCTGAGCTGATTGGGGCTATG
CTTGAACCCACTGATGAAGAGCCTAAAGAAGAGAAACCACCCACTAAAAGTCGGAATCCTACACCTGATCA
GAAGAGAGAAGATGATCAGGGTGCAGCTGAGATTCAAGTGCCTGACCTGGAAGCCGATCTCCAGGAGCTAT
GTCAGACAAAGACTGGGGATGGATGTGAAGGTGGTACTGATGTCAAGGGGAAGATTCTACCAAAAGCAGAG
CACTTTAAAATGCCAGAAGCAGGTGAAGGGAAATCACAGGTTTAAAGGAAGATAAGCTGAAACAACACAAA
CTGTTTTTATATTAGATATTTTACTTTAAAATATCTTAATAAAGTTTTAAGCTTTTCTCCAAAAAAAAAAA
AAA

The encoded protein having 111 amino acid residues is presented using the one-letter code in Table 4B (SEQ ID NO:10). The Psort profile for NOV4 predicts that this sequence is likely to be localized at the mitochondrial matrix space with a certainty of 0.4776. The Psort profile indicates that NOV4 is likely to have no N-terminal signal sequence. Based on Hydropathy plot, NOV4 contains no transmembrane domain.

TABLE 4B
Encoded NOV4 protein sequence (SEQ ID NO:10).

MSWRGRSTYRPRPRRSLQPPELIGAMLEPTDEEPKEEKPPTKSRNPTPDQ
KREDDQGAAEIQVPDLEADLQELCQTKTGDGCEGGTDVKGKILPKAEHFK
MPEAGEGKSQV

NOV4 was originally cloned from brain, fetal brain, pregnant uterus and placental JAR cells. Additional human tissues express identifiable SeqCalling™ fragments of NOV4. These tissues include pooled adrenal gland, placenta, pooled uterus, BeWo pool and brain.

In a search of the Patp database, which is a proprietary database that contains sequences published in patents and patent publications, NOV4 was identified as having 111 of 111 amino acid residue (100%) identical to, the 111 amino acid residue protein CCYPR-48, a human cell cycle and proliferation protein (Patp:AAB60500) (E Value=0). Table 4C shows the sequence alignment between NOV4 and CCYPR-48.

TABLE 4C
NOV4 alignment with CCYPR-48 (SEQ ID NO:65)

Score = 596 (209.8 bits), Expect = 3.5e − 57, P =°3.5e − 57
Identities = 111/111 (100%), Positives = 111/111 (100%), Frame = +3

| | | | | |
|---|---|---|---|---|
| Query: | 203 | MSWRGRSTYRPRPRRSLQPPELIGAMLEPTDEEPKEEKPPTKSRNPTPDQKREDDQGAAE | 386 | (SEQ ID NO:85) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1 | MSWRGRSTYRPRPRRSLQPPELIGAMLEPTDEEPKEEKPPTKSRNPTPDQKREDDQGAAE | 60 | (SEQ ID NO:66) |
| Query: | 387 | IQVPDLEADLQELCQTKTGDGCEGGTDVKGKILPKAEHFKMPEAGEGKSQV | 538 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 61 | IQVPDLEADLQELCQTKTGDGCEGGTDVKGKILPKAEHFKMPEAGEGKSQV | 111 | |

Possible SNPs found for NOV4 are listed in Table 4D.

TABLE 4D
cSNPs for NOV4

| Base Position | Wild | | Amino Acid |
|---|---|---|---|
| of cSNP | Type | Variant | Change |
| 255 | T | G | Leu -> Val |
| 243 | C | T | Pro -> Ser |
| 206 | T | A | None |

A BLAST search against public databases revealed that the disclosed NOV4 protein (SEQ ID NO:14) has 111 of 111 residues (100%) identical to an unnamed human protein (Gi|14328032|gb|AAH09232.1|AAH09232, E value=2e-31) that is similar to human G antigen 8. In addition, NOV4 also has homology to human Melanoma Associated Antigen GAGE-8 (ACC:076087, 117 aa, Expect=2.7e-17, Score=212 (74.6 bits).

This information is presented graphically in the multiple sequence alignment given in Table 4F (with NOV4 being shown on line 1) as a ClustalW analysis comparing NOV4 with the unnamed human that is similar to G antigen 8.

[image]

[image]

The similarity between the disclosed NOV4 and CCYPR-48, a human cell cycle and proliferation protein suggests that NOV4 may function as a member of a cell cycle and proliferation-like protein.

Cell division is the fundamental process by which all living things grow and reproduce. In unicellular organisms such as yeast and bacteria, each cell division doubles the number of organisms, while in multicellular species many rounds of cell division are required to replace cells lost by wear or by programmed cell death, and for cell differentiation to produce a new tissue or organ. Properly regulated cell division cycle is thus vital for many important biological processes, such as reproduction, differentiation and proliferation, apoptosis and aging and senescence. The consequences of defects in proper cell division cycle are diverse, depending on types of defects and types of cells in which the defects are located. For example, uncoordinated cell proliferation in many tissues can lead to formation of various forms of cancers. Not surprisingly, many oncoproteins are known to affect cell cycle controls, and many tumor-suppressor genes are also involved in regulating cell proliferation. For another example, defects in triggering apoptosis in immune cells that fail to distinguish self molecules from foreign molecules can lead to autoimmune disorders. In addition, failure to induce apoptosis in tumor cells and virus-infected cells may render an organism susceptible to tumors and infections.

Extensive sequence similarity exists between NOV4 and CCYPR-48, which is expressed in developmental tissues, suggesting that NOV4 may play a role in immune, developmental and cell signaling disorders, and cell proliferative disorders including cancer.

In addition, NOV4 is homologous to human Melanoma Associated Antigen GAGE-8. Many human tumors express antigens that are recognized in vitro by cytolytic T lymphocytes (CTLs) derived from the tumor-bearing patient. The GAGE (G antigen) gene family members encode such antigens. See OMIM 604132.

Taken together, the nucleic acids and proteins of NOV4 may be useful in potential therapeutic applications implicated in various immune, developmental and cell signaling disorders, and cell proliferative disorders including cancer. For example, a cDNA encoding the cell cycle and proliferation-like protein may be useful in gene therapy, and the cell cycle and proliferation-like protein may be useful when administered to a subject in need thereof. In the treatment of disorders associated with increased cell cycle and proliferation protein expression or activity, it is desirable to decrease the expression or activity of NOV4. In the treatment of disorders associated with decreased cell cycle and proliferation protein expression or activity, it is desirable to increase the expression or activity of NOV4. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The NOV4 compositions of the present invention will have efficacy for treatment of patients suffering from, for example, immune disorders, developmental disorders, cell-signaling disorders, cell proliferative disorders and cancers. Other pathologies and disorders are contemplated.

The novel nucleic acid encoding a cell cycle and proliferation-like protein, and the cell cycle and proliferation-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods and other diseases, disorders and conditions of the like. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below.

For example, the disclosed NOV4 protein has multiple hydrophilic regions, each of which can be used as an immunogen. In one embodiment, a contemplated NOV4 epitope is from about amino acids 8 to 25. In another embodiment, a NOV4 epitope is from about amino acids 25 to 65. In additional embodiments, NOV4 epitopes are from about amino acids 65 to 111. These novel proteins can also be used to develop assay system for functional analysis.

NOV5

A NOV5 sequence according to the invention includes a nucleic acid sequence encoding a polypeptide related to the cadherin family of proteins. A NOV5 nucleic acid and its encoded polypeptide includes the sequence shown in Tables 5A-5B. NOV5 nucleic acid and amino acid sequences are alternatively referred to as clone 34405797.0.15. A disclosed NOV5 nucleic acid of 3670 nucleotides is shown in Table 5A, and is identified as SEQ ID NO: 11. The disclosed NOV5 open reading frame (“ORF”) begins at the ATG initiation codon at nucleotides 50-52, shown in bold in Table 1A. The disclosed NOV5 ORF terminates at a TAG codon at nucleotides 3460-3462. Table 5A notes the putative untranslated regions 5′ to the start codon and 3′ to the stop codon with underlining, and the start and stop codons with bold lettering.

TABLE 5A
NOV5 Nucleotide Sequence

| | |
|---|---|
| CAATTGCTTTGCTGTTTTAACTTGCTCTGTGAGGGAAATCTCATAAACTGACCAATGCACCAAATGAATGCTAAAATGCA | (SEQ ID NO:11) |
| CTTTAGGTTTGTTTTTGCACTTCTGATAGTATCTTTCAACCACGATGTACTGGGCAAGAATTTGAAATACAGGATTTATG | |
| AGGAACAGAGGGTTGGATCAGTAATTGCAAGACTATCAGAGGATGTGGCTGATGTTTTATTGAAGCTTCCTAATCCTTCT | |
| ACTGTTCGATTTCGAGCCATGCAGAGGGGAAATTCTCCTCTACTTGTAGTAAACGAGGATAATGGGGAAATCAGCATAGG | |
| GGCTACAATTGACCGTGAACAACTGTGCCAGAAAAACTTGAACTGTTCCATAGAGTTTGATGTGATCACTCTACCCACAG | |
| AGCATCTGCAGCTTTTCCATATTGAAGTTGAAGTGCTGGATATTAATGACAATTCTCCCCAGTTTTCAAGATCTCTCATA | |
| CCTATTGAGATATCTGAGAGTGCAGCAGTTGGGACTCGCATTCCCCTGGACAGTGCATTTGATCCAGATGTTGGGGAAAA | |
| TTCCCTCCACACATACTCGCTCTCTGCCAATGATTTTTTTAATATCGAGGTTCGGACCAGGACTGATGGAGCCAAGTATG | |
| CAGAACTCATAGTGGTCAGAGAGTTAGATCGGGAGCTGAAGTCAAGCTACGAGCTTCAGCTCACTGCCTCAGACATGGGA | |
| GTACCTCAGAGGTCTGGCTCATCCATACTAAAAATAAGCATTTCAGACTCCAATGACAACAGCCCTGCTTTTGAGCAGCA | |
| ATCTTATATAATACAACTCTTAGAAAACTCCCCGGTTGGCACTTTGCTCTTAGATCTGAATGCCACGGATCCAGATGAGG | |
| GCGCTAATGGGAAAATTGTATATTCCTTCAGCAGTCATGTGTCTCCCAAAATTATGGAGACTTTTAAAATTGATTCTGAA | |
| AGAGGACATTTGACTCTTTTCAAGCAAGTGGATTATGAAATCACCAAATCCTATGAGATTGATGTTCAGGCTCAAGATTT | |
| GGGTCCAAATTCAATCCCAGCCCATTGCAAAATTATAATTAAGGTTGTGGATGTTAATGACAATAAACCTGAAATTAACA | |
| TCAACCTCATGTCCCCTGGAAAAGAAGAAATATCTTATATTTTTGAAGGGGATCCTATTGATACATTTGTTGCTTTGGTC | |
| AGAGTTCAGGACAAGGATTCTGGGCTGAATGGAGAAATAGTTTGTAAGCTTCATGGACATGGTCACTTTAAACTTCAGAA | |
| GACATATGAAAACAATTATTTAATCTTAACTAATGCCACACTGGATAGAGAAAAGAGATCTGAGTATAGTTTGACTGTAA | |
| TCGCTGAGGACAGGGGGACACCCAGTCTCTCTACAGTGAAACATTTTACAGTTCAAATCAATGATATCAATGACAATCCA | |
| CCCCACTTCCAGAGAAGCCGATATGAATTTGTAATTTCAGAAAATAACTCACCAGGGGCATATATCACCACTGTTACAGC | |
| CACAGATCCTGATCTTGGAGAAAATGGGCAAGTGACATACACCATCTTGGAGAGTTTTATTCTAGGAAGTTCCATAACTA | |
| CATATGTAACCATTGACCCATCTAATGGAGCCATCTATGCCCTCAGAATCTTTGATCATGAAGAAGTGAGTCAGATCACT | |
| TTTGTGGTAGAAGCAAGAGATGGAGGAAGCCCGAAGCAACTGGTAAGCAATACCACAGTTGTGCTCACCATCATTGACGA | |
| AAATGACAACGTTCCTGTGGTTATAGGGCCTGCATTGCGTAATAATACGGCAGAAATCACCATTCCCAAAGGGGCTGAAA | |
| GTGGCTTTCATGTCACAAGAATAAGGGCAATTGACAGAGACTCTGGTGTGAATGCTGAACTCAGCTGCGCCATAGTAGCA | |
| GGTAATGAGGAGAATATCTTCATAATTGATCCACGATCATGTGACATCCATACCAACGTTAGCATGGATTCTGTTCCCTA | |
| CACAGAATGGGAGCTGTCAGTTATCATTCAGGACAAAGGCAATCCTCAGCTACATACCAAAGTCCTTCTGAAGTGCATGA | |
| TCTTTGAATATGCAGAGTCGGTGACAAGTACAGCAATGACTTCAGTAAGCCAGGCATCCTTGGATGTCTCCATGATAATA | |
| ATTATTTCCTTAGGAGCAATTTGTGCAGTGTTGCTGGTTATTATGGTGCTATTTGCAACTAGGTGTAACCGCGAGAAGAA | |
| AGACACTAGATCCTATAACTGCAGGGTGGCCGAATCAACTTACCAGCACCACCCAAAAAGGCCATCCCGGCAGATTCACA | |
| AAGGGGACATCACATTGGTGCCTACCATAAATGGCACTCTGCCCATCAGATCTCATCACAGATCGTCTCCATCTTCATCT | |
| CCTACCTTAGAAAGAGGGCAGATGGGCAGCCGGCAGAGTCACAACAGTCACCAGTCACTCAACAGTTTGGTGACAATCTC | |
| ATCAAACCACGTGCCAGAGAATTTCTCATTAGAACTCACCCACGCCACTCCTGCTGTTGAGCAGGTCTCTCAGCTTCTTT | |
| CAATGCTTCACCAGGGGCAATATCAGCCAAGACCAAGTTTTCGAGGAAACAAATATTCCAGGAGCTACAGATATGCCCTT | |
| CAAGACATGGACAAATTTAGCTTGAAAGACAGTGGCCGTGGTGACAGTGAGGCAGGAGACAGTGATTATGATTTGGGGCG | |
| AGATTCTCCAATAGATAGGCTGTTGGGTGAAGGATTCAGCGACCTGTTTCTCACAGATGGAAGAATTCCAGCAGCTATGA | |
| GACTCTGCACGGAGGAGTGCAGGGTCCTGGGACACTCTGACCAGTGCTGGATGCCACCACTGCCCTCACCGTCTTCTGAT | |
| TATAGGAGTAACATGTTCATTCCAGGGGAAGAATTCCCAACGCAACCCCAGCAGCAGCATCCACATCAGAGTCTTGAGGA | |
| TGACGCTCAGCCTGCAGATTCCGGTGAAAAGAAGAAGAGTTTTTCCACCTTTGGAAAGGACTCCCCAAACGATGAGGACA | |
| CTGGGGATACCAGCACATCATCTCTGCTCTCGGAAATGAGCAGTGTGTTCCAGCGTCTCTTACCGCCTTCCCTGGACACC | |
| TATTCTGAATGCAGTGAGGTGGATCGGTCCAACTCCCTGGAGCGCAGGAAGGGACCCTTGCCAGCCAAAACTGTGGGTTA | |
| CCCACAGGGGGTAGCGGCATGGGCAGCCAGTACGCATTTTCAAAATCCCACCACCAACTGTGGGCCGCCACTTGGAACTC | |
| ACTCCAGTGTGCAGCCTTCTTCAAAATGGCTGCCAGCCATGGAGGAGATCCCTGAAAATTATGAGGAAGATGATTTTGAC | |
| AATGTGCTCAACCACCTCAATGATGGGAAACACGAACTCATGGATGCCAGTGAACTGGTGGCAGAGATTAACAAACTGCT | |
| TCAAGATGTCCGCCAGAGCTAGGAGATTTTAGCGAAGCATTTTTGTTTCCATGTATATGGAAATAGGGAACAACAACAAC | |
| AACAAAAAACCCTGAAAGAACTGGCATTGCCAAATAGTTGCATTTATCATAAATGTGTCTGTGTATATTGAATATTAAAT | |
| ACTGTATTTTCGTATGTACACAAAAAAAAAAAAAAAAAAAAAAAAAAAAAAAAAAAAAAAAAAAAAAAAG | |

The NOV5 protein encoded by SEQ ID NO:12 has 1135 amino acid residues and is presented using the one-letter code in Table 5B. The NOV5 polypeptide has a predicted molecular weight of 126.15 kDa. The Psort profile for NOV5 predicts that this sequence has a signal peptide and is likely to be localized at the plasma membrane with a certainty of 0.4600. The most likely cleavage site for a NOV5 peptide is between amino acids 27 and 28, i.e, at the slash in the amino acid sequence VLG-KN (underlined in Table 5B) based on the SignalP prediction results. NOV5 residues 675-710 are predicted to be the transmembrane domain and residues 710-1135 are predicted to form six domains, based on Hydropathy plot analysis.

TABLE 5B
Encoded NOV5 protein sequence

| | |
|---|---|
| MHQMNAKMHFRFVFALLEVSFNGDVLG/KNLKYRIYEEQRVGSVIARLSEDVADVLLKLPNPSTV | (SEQ ID NO:12) |
| RFRAMQRGNSPLLVVNEDNGEISIGATIDREQLCQKNLNCSIEFDVITLPTEHLQLFHIEVEVLD | |
| INDNSPQFSRSLIPIEISESAAVGTRIPLDSAFDPDVGENSLHTYSLSANDFFNIEVRTRTDGAK | |
| YAELIVVRELDRELKSSYELQLTASDMGVPQRSGSSILKISISDSNDNSPAFEQQSYIIQLLENS | |
| PVGTLLLDLNATDPDEGANGKIVYSFSSHVSPKIMETFKIDSERGHLTLFKQVDYEITKSYEIDV | |
| QAQDLGPNSIPAHCKIIIKVVDVNDNKPEININLMSPGKEEISYIFEGDPIDTFVALVRVQDKDS | |
| GLNGEIVCKLHGHGHFKLQKTYENNYLILTNATLDREKRSEYSLTVIAEDRGTPSLSTVKHFTVQ | |
| INDINDNPPHFQRSRYEFVISENNSPGAYITTVTATDPDLGENGQVTYTILESFILGSSITTYVT | |
| IDPSNGAIYALRIFDHEEVSQITFVVEARDGGSPKQLVSNTTVVLTIIDENDNVPVVIGPALRNN | |
| TAEITIPKGAESGFHVTRIRAIDRDSGVNAELSCAIVAGNEENIFIIDPRSCDIHTNVSMDSVPY | |
| TEWELSVIIQDKGNPQLHTKVLLKCMIFEYAESVTSTAMTSVSQASLDVSMIIIISLGAICAVLL | |
| VIMVLFATRCNREKKDTRSYNCRVAESTYQHHPKRPSRQIHKGDITLVPTINGTLPIRSHHRSSP | |
| SSSPTLERGQMGSRQSHNSHQSLNSLVTISSNHVPENFSLELTHATPAVEQVSQLLSMLHQGQYQ | |
| PRPSFRGNKYSRSYRYALQDMDKFSLKDSGRGDSEAGDSDYDLGRDSPISRLLGEGFSDLFLTDG | |
| RIPAAMRLCTEECRVLGHSDQCWMPPLPSPSSDYRSNMFIPGEEFPTQPQQQHPHQSLEDDAQPA | |
| DSGEKKKSFSTFGKDSPNDEDTGDTSTSSLLSEMSSVFQRLLPPSLDTYSECSEVDRSNSLERRK | |
| GPLPAKTVGYPQGVAAWAASTHFQNPTTNCGPPLGTHSSVQPSSKWLPAMEEIPENYEEDDFDNV | |
| LNHLNDGKHELMDASELVAEINKLLQDVRQS | |

NOV5 was originally cloned from fetal kidney tissue. Additional human tissues express identifiable SeqCalling™ fragments of NOV5. These tissues include bone, NHFLS, HCN, HFLSRA, kidney bone marrow, HFDPC, hair follicles, salivary gland, and NHMC-RM.

NOV5 is a cadherin-like protein. Cadherins are calcium-dependent adhesive proteins that mediate cell-to-cell interaction. See, e.g., Online Mendelian Inheritance in Man (“OMIM”) Identity. No. 601120 at URL http://www.ncbi.nlm.nih.gov/entrez/query.fcgi?db=OMIM. Cadherins constitute an expanding family of receptors involved in the structural and functional organization of cells in various tissues. See, e.g., Huber et al., 1996 Genomics 32: 21-28. Members of the family include epithelial cadherin (E-cadherin; OMIM ID. 192090), neural cadherin (N-cadherin; OMIM ID. 114020), placental cadherin (P-cadherin; OMIM ID. 114021), muscle cadherin (M-cadherin; OMIM ID. 114019), and vascular endothelial cadherin (VE-cadherin, or CDH5). Family members share a common domain structure and primary sequence homologies. Each cadherin type has a unique tissue-distribution pattern. In addition, multiple cadherin types may be found at the surface of a particular cell. See, e.g., Salomon et al., 1992 J. Cell Sci. 102: 7-17.

The full NOV5 amino acid sequence of the protein of the invention has 1102 of 1135 amino acid residues (97%) identical to, and positive with, the 1136 amino acid residue KIAA1562 protein from Homo sapiens (gi|10047189|dbj|BAB13388.1|(AB046782)) (E=0.0). Further, the NOV5 polypeptide has 679 of 709 amino acid residues (95%) identical to, and 676 of 709 residues (95%) positive with, the 709 amino acid residue DKFZp434B0923.1 hypothetical protein from Homo sapiens ( gi|1359910|pir||T46413) (E=0.0); and 71 of 242 amino acid residues (29%) identical to, and 116 of 242 residues (47%) positive with, a second hypothetical protein from Homo sapiens (gi|6808080|emb|CAB70755.1|(AL137471)) (E=2e⁻²¹). NOV5 protein has 390 of 1044 amino acid residues (37%) identical to, and 577 of 1044 residues (54%) positive with, the 1044 amino acid residue OL-protocadherin protein isoform from Mus musculus (gi|14210851|gb|AAK57195.1|AF334801_{—}1(AF334801)) (E=1e⁻¹⁸⁰) where Gaps=129/1044 (12%). NOV5 protein has 390 of 1038 amino acid residues (37%) identical to, and 574 of 1038 residues (54%) positive with, the 1093 amino acid residue KIAA1400 protein from Homo sapiens ( gi|7243181|dbj|BAA92638.1|(AB037821)) (E=1e⁻¹⁷⁹), where Gaps=129/1038 (12%). NOV5 protein has 1135 of 1135 amino acid residues (100%) identical to, and positive with, the 1135 amino acid residue qs14_{—}3 protein sequence from Homo sapiens (PCT Publication WO200009552-A1; patp accno:AAY94923) (E=0.0). NOV5 protein has 460 of 461 amino acid residues (99%) identical to, and positive with, the 461 amino acid residue vc35_{—}1 protein from Homo sapiens (PCT Publication WO200011015-A1; patp accno: AAY94991) (E=7.0e⁻²⁴⁹).

Any reference to NOV5 is assumed to encompass all variants.

The disclosed NOV5 protein has good identity with a number of cadherin proteins. The identity information used for ClustalW analysis is presented in Table 5C.

TABLE 5C
BLAST results for NOV5

| Gene Index/ | Protein/ | Length | Identity | Positives | |
|---|---|---|---|---|---|
| Identifier | Organism | (aa) | (%) | (%) | Expect |
| gi\|10047189\|dbj\|BAB | KIAA1562 | 1136 | 1102/1135 | 1102/1135 | 0.0 |
| 13388.1\| (AB046782) | protein Homo | | (97%) | (97%) | |
| | sapiens | | | | |
| gi\|11359910\|pir\|\|T4 | hypothetical | 709 | 676/709 | 676/709 | 0.0 |
| 6413 | protein | | (95%) | (95%) | |
| gi\|6808080\|emb\|CAB7 | DKFZp434B0923 | | | | |
| 0755.1\| (AL137471) | .1 Homo | | | | |
| | sapiens | | | | |
| gi\|14210851\|gb\|AAK5 | OL- | 1040 | 91/1044 | 577/1044 | e-180 |
| 7195.1\|AF334801 1 | protocadherin | | (37%) | (54%) | |
| (AF334801) | isoform Mus | | | | |
| | musculus | | | | |
| gi\|7243181\|dbj\|BAA9 | KIAA1400 | 1093 | 390/1038 | 574/1038 | e-179 |
| 2638.1\| (AB037821) | protein Homo | | (37%) | (54%) | |
| | sapiens | | | | |
| gi\|14210853\|gb\|AAK5 | OL- | 1030 | 390/1028 | 575/1028 | e-177 |
| 7196.1\|AF334802 1 | protocadherin | | (37%) | (54%) | |
| (AF334802) | isoform | | | | |
| | Gallus gallus | | | | |

This information is presented graphically in the multiple sequence alignment given in Table 5D (with NOV5 being shown on line (1) as a ClustalW analysis comparing NOV5 with related protein sequences.

[image]

[image]

[image]

[image]

DOMAIN results for NOV5 were collected from the Conserved Domain Database (CDD) with Reverse Position Specific BLAST. This BLAST samples domains found in the Smart and Pfam collections. The results for NOV5 are listed in Table 5E with the statistics and domain description.

The NOV5 protein region from amino acid residue 59 through 672 are strongly predicted (E=9e⁻⁴ to 7e⁻¹⁷) to contains six “CA, cadherin repeats” (SEQ ID NO:73, see Table 5F) and five “cadherin domain” regions of homology (SEQ ID NO:74, see Table 5G). As shown in Table 5E, below, the DOMAIN type (column 1) and residues (column 2) aligned with the designated NOV5 residues (column 3) with the corresponding Score (column 4) and E values (column 5).

TABLE 5E
DOMAIN results for NOV5

| | DOMAIN | | Score | |
|---|---|---|---|---|
| DOMAIN type | residues | NOV5 residues | (bits) | E value |
| CA, Cadherin repeats | 9-82 | 59-135 | 39.7 | 9e-04 |
| CA, Cadherin repeats | 2-82 | 160-244 | 72.8 | 9e-14 |
| CA, Cadherin repeats | 1-82 | 268-352 | 80.1 | 6e-16 |
| CA, Cadherin repeats | 1-82 | 382-463 | 79.3 | 1e-15 |
| CA, Cadherin repeats | 1-82 | 487-574 | 83.2 | 7e-17 |
| CA, Cadherin repeats | 1-70 | 603-672 | 55.5 | 2e-08 |
| Cadherin domain | 2-91 | 143-236 | 61.6 | 2e-10 |
| Cadherin domain | 1-91 | 251-344 | 76.6 | 6e-15 |
| Cadherin domain | 7-91 | 371-455 | 63.5 | 6e-11 |
| Cadherin domain | 1-92 | 470-567 | 68.6 | 2e-12 |
| Cadherin domain | 3-87 | 588-672 | 58.9 | 1e-09 |

TABLE 5F
Amino Acid sequence for CA (smart accno. 00112)

| | |
|---|---|
| VSATDADSGENGKVTYSILSGNDGGLFSIDPETGIITTTKPLDREEQSEYTLTVEATDGGGPPLSST | (SEQ ID NO:73) |
| ATVTVTVLDVNDNAP | |

TABLE 5G
Amino Acid sequence for the cadherin domain (pfam accno. 00028)

| | |
|---|---|
| YSASVPENAPVGTEVLTVTATDADLGPNGRIFYSILGGGPGGWFRIDPDTGDLSTTKPLDRESIGEY | (SEQ ID NO:74) |
| ELTVLATDSGGPPLSGTTTVTITVL | |

Cadherins are a family of animal glycoproteins responsible for calcium-dependent cell-cell adhesion. See, e.g., Takeichi 1987 Trends Genet. 2: 213-217; Takeichi 1990 Annu. Rev. Biochem. 59: 237-252. Cadherins preferentially form homomeric complexes between connecting cells; thus acting as both receptor and ligand. Several different isoforms are distributed in a tissue-specific manner in a wide variety of organisms. Cells containing different cadherins tend to segregate in vitro, while those that contain the same cadherins tend to preferentially aggregate together. This observation is linked to the finding that cadherin expression causes morphological changes involving the positional segregation of cells into layers, suggesting they may play an important role in the sorting of different cell types during morphogenesis, histogenesis and regeneration. They may also be involved in the regulation of tight and gap junctions, and in the control of intercellular spacing. Cadherins are evolutionarily related to the desmogleins, which are components of intercellular desmosome junctions involved in the interaction of plaque proteins.

Cadherins are glycoproteins involved in Ca²⁺-mediated cell-cell adhesion. Cadherin domains occur as repeats in the extracellular regions that are thought to mediate cell-cell contact when bound to calcium. Structurally, cadherins comprise a number of domains: these include a signal sequence; a propeptide of around 130 residues; an extracellular domain of around 600 residues; a single transmembrane domain; and a well-conserved C-terminal cytoplasmic domain of about 150 residues. The extracellular domain can often be subdivided into 5 parts, 4 of which are repeats of about 110 residues, and the fifth contains 4 conserved cysteines. This pattern is thought to include two conserved aspartic acid residues as well as two asparagines. See, generally the PROSITE entries PDOC00205, PS00232, PS50268, available at http://expasv.ch, and InterPro entries IPR000233 and IPR002126 available at http://wwv.ebi.ac.uk/interpro. The calcium-binding region of cadherins is thought to be located in the extracellular domain.

Included in this family are DSG2human: Desmoglein 2 (SwissProt No. Q14126); CADF_human: Muscle (M-cadherin) (CDHI4) (SwissProtNo. P55291); CADD_human: T-cadherin (truncated cadherin) (CDH13) (SwissProt No. P55290); CAD5_human: Vascular endothelial (VE-cadherin) (CDH5) (SwissProt No. P33151); CAD3_human: Placental (P-cadherin) (CDH3) (SwissProt No. P22223); DSG3_human: Desmoglein 3 (Pemphigus vulgaris antigen) (SwissProt No. P32926); CADC_human: Brain (BR-cadherin) (CDH12) (SwissProt No. P55289); CADB_human: Osteoblast (OB-cadherin) (CDH11) (SwissProt No. P55287); CAD8_human: Cadherin-8 (CDH8) (SwissProt No. P55286); CAD6_human: Kidney (K-cadherin) (CDH6) (SwissProt No. P55285); CAD4_human: Retinal (R-cadherin) (CDH4) (SwissProt No. P55283); CADH_rat: Liver-intestine (LI-cadherin) (SwissProt No. P55281); CADF_Xenopus laevis: EP-cadherin (SwissProt No. P33148); CAD1_human: Epithelial (E-cadherin) (a.k.a. uvomorulin or L-CAM) (CDH1) (SwissProt No. P12830); DSG1_human: Desmoglein 1 (desmosomal glycoprotein 1) (SwissProt No. Q02413); and CAD2_HUMAN: Neural (N-cadherin) (CDH2) (SwissProt No. P19022).

The nucleic acids and proteins of NOV5 have biological activities that would make them suitable for treating, preventing or ameliorating medical conditions in humans and animals; and so are useful in potential therapeutic applications implicated in various pathological disorders, described further below. For example, a cDNA encoding the cadherin-like protein is useful in gene therapy, and the vascular cadherin-like protein is useful when administered to a subject in need thereof. The NOV5 polynucleotides can be used as markers for tissues in which the protein is preferentially expressed, as molecular weight markers on Southern gels. NOV5 nucleic acid sequences of the invention can be used as chromosome markers or tags to identify chromosomes or to map gene positions, and as a source of diagnostic primers and probes.

The secreted NOV5 proteins of the invention include those that are thought to be only partially secreted, i.e., transmembrane proteins. The proteins of the invention may exhibit one or more activities selected from the following: cytokine activity; cell proliferation; virucide; antibacterial; antifungal; anti-inflammatory; dermatological; antidiabetic; antiasthmatic; antiarthritic; antirheumatic; protozoacide; antithyroid; tumor inhibitor; growth stimulant, hematopoietic stimulant; contraceptive; differentiation; immune modulation (i.e., immunostimulant; immunosuppressant); haematopoiesis regulation; tissue growth modulatory activity; activin/inhibin activity; chemotactic/chemokinetic activity; haemostatic and thrombolytic activity; anti-inflammatory activity; and tumor inhibition activity. The proteins may be administered to patients as vaccines, and the nucleotides may be used as part of a gene therapy regime.

NOV5 can be used in the treatment of immune deficiencies and disorders, such as severe combined immunodeficiency (SCID), as well as viral, bacterial, fungal and other infections. These infections include human immunodeficiency virus (HIV), hepatitis, herpes viruses, mycobacteria, Leismania species, malaria and candidiasis. NOV5 proteins can be used to treat autoimmune disorders such as connective tissue disease, multiple sclerosis, systemic lupus erythematosis, rheumatoid arthritis, autoimmune pulmonary inflammation, Guillain-Barre syndrome, autoimmune thyroiditis, insulin dependent diabetes mellitus, myasthenia gravis, graft-versus-host-disease and autoimmune inflammatory eye disease. NOV5 proteins can also be used to treat allergic conditions, such as asthma and anemia. NOV5 proteins can additionally be used to treat cancer; cardiovascular disorders; blood disorders; hemophilia; neurodegenerative disease; genetic disorders; hemophilia; cardiovascular diseases; cancer; bacterial, fungal and viral infections, especially HIV. In various further embodiments, NOV5 can be used for treating wounds, burns, ulcers, osteoporosis, osteoarthritis, periodontal diseases, Alzheimer's disease, Parkinson's disease, Huntington's disease and amyotrophic lateral sclerosis (“ALS”). NOV5 proteins with activin/inhibin activity may additionally be useful as contraceptives.

The polypeptides can be used as immunogens to produce antibodies specific for the invention, and as vaccines. They can also be used to screen for potential agonist and antagonist compounds. For example, a cDNA encoding the cadherin-like protein may be useful in gene therapy, and the receptor-like protein may be useful when administered to a subject in need thereof. The novel nucleic acid encoding cadherin-like protein, and the cadherin-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV5 protein has multiple hydrophilic regions, each of which can be used as an immunogen. in one embodiment, a contemplated NOV5 epitope is from about amino acids 10 to 40. In another embodiment, a NOV5 epitope is from about amino acids 110 to 130. In additional embodiments, NOV5 epitopes are from amino acids 150 to 175, 190 to 200, 240-270 and from amino acids 280 to 320. This novel protein also has value in development of powerful assay system for functional analysis of various human disorders, which will help in understanding of pathology of the disease and development of new drug targets for various disorders.

Taqman data for NOV5 is included in Example 1.

NOV6

NOV 6 represents novel members of a family of related lysozyme C-1 precursor-like proteins, which are related to the glycoside hydrolase family. Included within NOV6 are four different family members designated NOV6a, NOV6b, NOV6c, and NOV6d. Each of these is discussed in detail below.

NOV6a 1-3

Disclosed herein are three related members of the NOV6 family, designated NOV6a1, NOV6b1, and NOV6c1. The nucleotide sequences of each of these NOV6as differ, but each of NOV6a 1, 2, and 3 code for the same protein. These sequences are discussed in detail below.

NOV6a1

NOV6a1 was initially identified by searching CuraGen's Human SeqCalling database for DNA sequences that translate into proteins with similarity to a protein family of interest. NOV6 a was derived by laboratory cloning of cDNA fragments covering the full length and/or part of the DNA sequence of the invention, and/or by in silico prediction of the full length and/or part of the DNA sequence of the invention from public human sequence databases. The laboratory cloning was performed using one or more of the methods summarized below:

SeqCalling™ Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, or steroids. The cDNA thus derived was then sequenced using CuraGen's proprietary SeqCalling technology.

RACE: Techniques based on the polymerase chain reaction such as rapid amplification of cDNA ends (RACE) were used to isolate or complete the predicted sequence of the cDNA of the invention. Usually multiple clones were sequenced from one or more human samples, to derive the sequences for fragments. Human samples from different donors from testis and fetal brain were used for the RACE reaction.

Sequence traces were evaluated manually and edited for corrections if appropriate. Fragment sequences were assembled with other fragments derived by RACE and by SeqCalling and with public ESTs using bioinformatics programs and were included in CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

SeqCalling assembly sequences were initially identified by searching CuraGen Corporation's Human SeqCalling database for DNA sequences which translate into proteins with similarity to LYSOZYME C-1 PRECURSOR and/or members of the LYSOZYME C-1 PRECURSOR family. One or more SeqCalling assemblies in 144861150 were identified as having suitable similarity. One or more of these assemblies were analyzed further to identify any open reading frames encoding novel full length proteins as well as novel splice forms of these genes. The resulting DNA sequence and protein sequence for a novel LYSOZYME C-1 PRECURSOR-like gene or one of its splice forms are reported here as CuraGen Ace. No. 5603288.0.20_da1, or NOV6a1.

The regions defined by all approaches were then manually integrated and manually corrected for apparent inconsistencies that may have arisen, for example, from miscalled bases in the original fragments used, or from discrepancies between predicted homology to a protein of similarity to derive the final sequence of the NOV6a1 reported here. When necessary, the process to identify and analyze SeqCalling assemblies, ESTs and genomic clones was reiterated to derive the full length sequence.

The disclosed novel NOV6a1 nucleic acid of 907 nucleotides (designated 5603288.0.20da1 or NOV6a1) is shown in Table 6A. An open reading begins with an ATG initiation codon at nucleotides 311-313 and ends with a TGA codon at nucleotides 788-790.

TABLE 6A
NOV6a1 Nucleotide Sequence (SEQ ID NO:13)

| | |
|---|---|
| CCCTCCTGGCTGCTCACGGCACGGCCTTCCCTCTGGCGCTTCCATTCTCCCCATCCTAATACG | (SEQ ID NO:13) |
| ACTCACTATAGGGCTCGAGCGGCCGCCCGGGCAGGTAAAAGATTCTAGCAGGCGGCAATTTCG | |
| CACTTTGAACTTGGAGGGCAGCAACAGAGTTGCAGGTGTAAAATAACGGGAAGGCGGGATGCG | |
| TGGCTAAATTGCTCTGCGTGCACAAAGAGTAGGAGAGCCCAGAGTTCCAGAATGCCCCTAATT | |
| CCGAACACCACAGGGTGAGTCTGGAGCAAGTCACCTGGGAGGGCTTACAGGTGCCATAATGAA | |
| GGCCTGGGGCACTGTGGTAGTGACCTTGGCCACGCTGATGGTTGTCACTGTGGATGCCAAGAT | |
| CTATGAACGCTGCGAGCTGGCGGCAAGACTGGAGAGAGCAGGGCTGAACGGCTACAAGGGCTA | |
| CGGCGTTGGAGACTGGCTGTGCATGGCTCATTATGAGAGTGGCTTTGACACCGCCTTCGTGGA | |
| CCACAATCCTGATGGCAGCAGTGAATATGGCATTTTCCAACTGAATTCTGCCTGGTGGTGTGA | |
| CAATGGCATTACACCCACCAAGAACCTCTGCCACATGGATTGTCATGACCTGCTCAATCGCCA | |
| TATTCTGGATGACATCAGGTGTGCCAAGCAGATTGTGTCCTCACAGAATGGGCTTTCTGCCTG | |
| GACTTCTTGGAGGCTACACTGTTCTGGCCATGATTTATCTGAATGGCTCAAGGGGTGTGATAT | |
| GCATGTGAAAATTGATCCAAAAATTCATCCATGACTCAGATTCGAAGAGACAGATTTTATCTT | |
| CCTTTCATTTCTTTCTCTTGTGCATTTAATAAAGGATGGTATCTATAAACAATGCAAAAAAAA | |
| AAAAAAAAAAAAAAAAAAAAAAAAA | |

The disclosed nucleic acid sequence has 263 of 420 nucleotides (62%) identical to a 603 base pair mRNA from Presbytis entellus (GENBANK-ID:PELYSOC|acc:X60235.1 mRNA P.entellus mRNA for lysozyme C), E value=3.5e-¹⁶).

The NOV6a protein encoded by SEQ ID NO:13 has 159 amino acid residues, and is presented using the one-letter code in Table 6B (SEQ ID NO:14). The SignalP, Psort and/or Hydropathy profile for NOV6a predict that NOV6a is likely to be localized extracellularly with a certainty of 0.6850, or the endoplasmic reticulum, with a certainty of 0.6400. A cleavage site is indicated at the slash in the sequence VDA-KI, between amino acids 21 and 22 in Table 6B. The hydropathy profile of the NOV6a lysozyme precursor-like protein indicates that this sequence has a strong signal peptide, supporting extracellular localization.

TABLE 6B
Encoded NOV6a protein sequence.

(SEQ ID NO:14)

MKAWGTVVVTLATLMVVTVDA/KIYERCELAARLERAGLNGYKGYGVGDW
LCMAHYESGFDTAFVDHNPDGSSEYGIFQLNSAWWCDNGITPTKNLCHMD
CHDLLNRHILDDIRCAKQIVSSQNGLSAWTSWRLHCSGHDLSEWLKGCDM
HVKIDPKIHP

The full amino acid sequence of the disclosed NOV6a protein was found to have 75 of 147 amino acid residues (51%) identical to, and 103 of 147 amino acid residues (70%) similar to, the 147 amino acid residue protein lysozyme C-I precursor protein from Anas platyrhynchos (domestic duck): ptnr:SWISSPROT-ACC:P00705 LYSOZYME C-1 PRECURSOR (EC 3.2.1.17) (1,4-BETA-N-ACETYLMURAMIDASE C), Expect=5.8e-40.

NOV6a2

NOV6a2 was initially identified by searching CuraGen's Human SeqCalling database for DNA sequences that translate into proteins with similarity to a protein family of interest.

The cDNA coding for the NOV6a2 (CG52754-03) sequence was cloned by the polymerase chain reaction (PCR) using the following primers: ACTATGGAAAATTTGAACACCAGTTC (SEQ ID NO:75) and CTATGCTGAGTCTGTGCTCCTG (SEQ ID NO:76). Primers were designed based on in silico predictions of the full length or some portion (one or more exons) of the cDNA/protein sequence of the invention. These primers were used to amplify a cDNA from a pool containing expressed human sequences derived from the following tissues: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea and uterus.

Multiple clones were sequenced and these fragments were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

The PCR product derived by exon linking, covering the entire open reading frame, was cloned into the pCR2.1 vector from Invitrogen to provide clone 123499::GMAC044846_A.698445.C13.

The novel nucleic acid of 506 nucleotides (designated CG52754-03 or NOV6a2) encoding a novel Lysozyme C-1 Precursor-like protein is shown in Table 6C. An open reading frame was identified beginning at nucleotides 5-7 and ending at nucleotides 482-484. The start and stop codons of the open reading frame are highlighted in bold type in the Table. Putative untranslated regions (underlined), if any, are found upstream from the initiation codon and downstream from the termination codon. In a search of sequence databases, it was found, for example, that the NOV6a2 nucleic acid sequence of this invention has 263 of 420 bases (62%) identical to a gb:GENBANK-ID:PELYSOC|acc:X60235.1 mRNA from Presbytis entellus (P.entellus mRNA for lysozyme c), E=3.9 e-16.

TABLE 6C
NOV6a2 Nucleotide Sequence (SEQ ID NO:15)

| | |
|---|---|
| CATAATGAAGGCCTGGGGCACTGTGGTAGTGACCTTGGCCACGCTGATGGTTGTCACTGT | (SEQ ID NO:15) |
| GGATGCCAAGATCTATGAACGCTGCGAGCTGGCGGCAAGACTGGAGAGAGCAGGGCTGAA | |
| CGGCTACAAGGGCTACGGCGTTGGAGACTGGCTGTGCATGGCTCATTATGAGAGTGGCTT | |
| TGACACCGCCTTCGTGGACCACAATCCTGATGGCAGCAGTGAATATGGCATTTTCCAACT | |
| GAATTCTGCCTGGTGGTGTGACAATGGCATTACACCCACCAAGAACCTCTGCCACATGGA | |
| TTGTCATGACCTGCTCAATCGCCATATTCTGGATGACATCAGGTGTGCCAAGCAGATTGT | |
| GTCCTCACAGAATGGGCTTTCTGCCTGGACTTCTTGGAGGCTACACTGTTCTGGCCATGA | |
| TTTATCTGAATGGCTCAAGGGGTGTGATATGCATGTGAAAATTGATCCAAAAATTCATCC | |
| ATGACTCAGATTCGAAGAGACAGATA | |

This nucleic acid sequence differs firom that of NOV6a1 by having 306 fewer nucleotides at the 5′ end, a base change from T to A (at position 812 of NOV6al or 506 of NOV6a2) and 95 fewer nucleotides at the 3′ end. The encoded NOV6a protein is the same.

The disclosed lysozyme c-1 precursor-like gene is expressed in at least the following tissues: cartilage, spleen, lung, kidney, white blood cells, plasma, saliva, milk and tears. Expression information was derived from the tissue sources of the sequences that were included in the derivation of the sequence of CuraGen Acc. No. CG52754-03.

The PSORT data suggests that theNOV6a2 lysozyme c-1 precursor-like protein may be localized at the plasma membrane and that the protein of CuraGen Acc. No. CG52754-03 is similar to the lysozyme c-1 precursor family, some members of which are secreted. Therefore it is likely that this protein is localized to the same sub-cellular compartment.

NOV6a3

NOV6a3 was identified by subjecting a previously identified clone, 30412306_{—}0_{—}100_dal (B), see NOV6d, below, to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated Accession Number 30412306_{—}0_{—}100_dal (NOV6a3).

The cDNA coding for the sequence was cloned by polymerase chain reaction (PCR) using the following primers: TGACCTTGGCCACGCTGATG (SEQ ID NO:77) and TCAATTTTCACATGCATATCACACCCC (SEQ ID NO:78) on the following pools of human cDNAs: Pool 1—Adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus.

Primers were designed based on in silico predictions for the full length or part (one or more exons) of the DNA/protein sequence of the invention or by translated homology of the predicted exons to closely related human sequences or to sequences from other species. Usually multiple clones were sequenced to derive the sequence which was then assembled similar to the SeqCalling process. In addition, sequence traces were evaluated manually and edited for corrections if appropriate.

Physical clone: The PCR product derived by exon linking was cloned into the pCR2.1 vector from Invitrogen. The bacterial clone 118885::30412306_{—}0_{—}100_dal.698324.C1 has an insert covering the entire open reading frame cloned into the pCR2.1 vector from Invitrogen.

The novel nucleic acid of 487 nucleotides (designated 30412306_{—}0_{—}100_dal or NOV6a3) encoding a novel Lysozyme C-1 Precursor-like protein is shown in Table 6D. An open reading frame was identified beginning with an ATG at nucleotides 1-3 and ending with a TGA at nucleotides 478-480. In a search of sequence databases, it was found, for example, that the NOV6a3 nucleic acid sequence of this invention has 263 of 420 bases (62%) identical to a gb:GENBANK-ID:PELYSOC|acc:X60235.1 mRNA from Presbytis entellus (P.entellus mRNA for lysozyme c), E=3.5 e-16.

TABLE 6D
NOV6a3 Nucleotide Sequence (SEQ ID NO:16)

| | |
|---|---|
| ATGAAGGCCTGGGGCACTGTGGTAGTGACCTTGGCCACGCTGATGGTTGTCACTGTGGATGCC | (SEQ ID NO:16) |
| AAGATCTATGAACGCTGCGAGCTGGCGGCAAGACTGGAGAGAGCAGGGCTGAACGGCTACAAG | |
| GGCTACGGCGTTGGAGACTGGCTGTGCATGGCTCATTATGAGAGTGGCTTTGACACCGCCTTC | |
| GTGGACCACAATCCTGATGGCAGCAGTGAATATGGCATTTTCCAACTGAATTCTGCCTGGTGG | |
| TGTGACAATGGCATTACACCCACCAAGAACCTCTGCCACATGGATTGTCATGACCTGCTCAAT | |
| CGCCATATTCTGGATGACATCAGGTGTGCCAAGCAGATTGTGTCCTCACAGAATGGGCTTTCT | |
| GCCTGGACTTCTTGGAGGCTACACTGTTCTGGCCATGATTTATCTGAATGGCTCAAGGGGTGT | |
| GATATGCATGTGAAAATTGATCCAAAAATTCATCCATGACTCAGAT | |

This nucleic acid sequence differs from that of NOV6a1 by having 310 fewer nucleotides at the 5′ end and 110 fewer nucleotides at the 3′ end. The encoded NOV6a protein is the same.

The following SNPs have been identified for NOV6a3: In the following positions, one or more consensus positions (Cons. Pos.) of the nucleotide sequence have been identified as SNPs. “Depth” represents the number of clones covering the region of the SNP. The Putative Allele Frequency (Putative Allele Freq.) is the fraction of all the clones containing the SNP. A dash (“-”), when shown, means that a base is not present. The sign “>” means “is changed to”.

TABLE 6E
NOV6a3 SNPs

| | |
|---|---|
| Cons.Pos.: 14 Depth: 49 | Cons.Pos.: 230 Depth: 68 Change: T>C |
| Change: G>− | Putative Allele Freq.: 0.029 |
| Putative Allele Freq.: 0.041 | |
| Cons.Pos.: 142 Depth: 70 | Cons.Pos.: 255 Depth: 68 Change: T>C |
| Change: G>A | Putative Allele Freq.: 0.029 |
| Putative Allele Freq.: 0.029 | |
| Cons.Pos.: 176 Depth: 69 | Cons.Pos.: 343 Depth: 69 Change: I>C |
| Change: C>T | Putative Allele Freq.: 0.029 |
| Putative Allele Freq.: 0.029 | |
| Cons.Pos.: 206 Depth: 69 | Cons.Pos.: 375 Depth: 69 Change: G>A |
| Change: T>A | Putative Allele Freq.: 0.029 |
| Putative Allele Freq.: 0.029 | |
| Cons.Pos.: 207 Depth: 69 | ConsPos.: 437 Depth: 69 Change: A>− |
| Change: G>A | Putative Allele Freq.: 0.029 |
| Putative Allele Freq.: 0.029 | |

The disclosed NOV6a3 Lysozyme C-1 Precursor-like protein is expressed in at least the following tissues: cartilage, spleen, lung, kidney, white blood cells, plasma, saliva, milk and tears. This information was derived by determining the tissue sources of the sequences that were included in the invention including but not limited to SeqCalling sources, Public EST sources, Literature sources, and/or RACE sources.

NOV6b

NOV6b is a novel member of the lysozyme C-1 precursor-like family of proteins, which are related to the glycoside hydrolase family. The cDNA coding for the NOV6b (5603288.0.1, CG52754-01) sequence was cloned by the polymerase chain reaction (PCR). Primers were designed based on in silico predictions of the full length or some portion (one or more exons) of the cDNA/protein sequence of the invention. These primers were used to amplify a cDNA from a pool containing expressed human sequences derived from fetal brain and kidney tissue.

Multiple clones were sequenced and these fragments were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed. Such sequences were included in the derivation of NOV6b only when the extent of identity in the overlap region with one or more SeqCalling assemblies was high. The extent of identity may be, for example, about 90% or higher, preferably about 95% or higher, and even more preferably close to or equal to 100%. When necessary, the process to identify and analyze SeqCalling fragments and genomic clones was reiterated to derive the full length sequence.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence. The Seq Calling Fragments of the clone were provided by the following human tissues: 10 human total RNAs from Clonetech (brain, fetal brain, liver, fetal liver, skeletal muscle, pancreas, kidney, heart, lung, and placenta.

The DNA sequence for the disclosed lysozyme C-1 precursor-like gene is reported here as CuraGen Acc. No.5603288.0.1, CG52754-01, or NOV6b. The disclosed novel NOV6b nucleic acid of 646 nucleotides (SEQ ID NO:17) is shown in Table 6F. An open reading frame begins at nucleotide 83 and ends with at nucleotide 559.

NOV6b differs from NOV6a1 in the following ways: NOV6b has 228 fewer nucleotides at the 5′ UTR and 33 fewer nucleotides at the 3′ UTR and one base change: G429 >T (numbered with respect to NOV6a1), resulting in a one amino acid change: G40>V. Other than this one amino acid change, NOV6a and NOV6b proteins are the same.

TABLE 6F
NOV6b Nucleotide Sequence (SEQ ID NO:17)

| | |
|---|---|
| CAGAGTTCCAGAATGCCCCTAATTCCGAACACCACAGGGTGAGTCTGGAGCAAGTCACCTG | (SEQ ID NO:17) |
| GGAGGGCTTACAGGTGCCATAATGAAGGCCTGGGGCACTGTGGTAGTGACCTTGGCCACGC | |
| TGATGGTTGTCACTGTGGATGCCAAGATCTATGAACGCTGCGAGCTGGCGGCAAGACTGGA | |
| GAGAGCAGGGCTGAACGTCTACAAGGGCTACGGCGTTGGAGACTGGCTGTGCATGGCTCAT | |
| TATGAGAGTGGCTTTGACACCGCCTTCGTGGACCACAATCCTGATGGCAGCAGTGAATATG | |
| GCATTTTCCAACTGAATTCTGCCTGGTGGTGTGACAATGGCATTACACCCACCAAGAACCT | |
| CTGCCACATGGATTGTCATGACCTGCTCAATCGCCATATTCTGGATGACATCAGGTGTGCC | |
| AAGCAGATTGTGTCCTCACAGAATGGGCTTTCTGCCTGGACTTCTTGGAGGCTACACTGTT | |
| CTGGCCATGATTTATCTGAATGGCTCAAGGGGTGTGATATGCATGTGAAAATTGATCCAAA | |
| AATTCATCCATGACTCAGATTCGAAGAGACAGATTTTATCTTCCTTTCATTTCTTTCTCTT | |
| GTGCATTTAATAAAGGATGGTATCTATAAACAATGC | |

NOV6b most likely has a cleavage site between positions 21 and 22, as indicated by the slash between VDA/KI in Table 6G.

TABLE 6G
Encoded NOV6b protein sequence (SEQ ID NO:18).

(SEQ ID NO:18)

MKAWGTVVVTLATLMVVTVDA/KIYERCELAARLERAGLNVYKGYGVGDW
LCMAGYESGFDTAFVDHNPDGSSEYGIFQLNSAWWCDNGITPTKNLCHMD
CHDLLNRHILDDIRCAKQIVSSQNGLSAWTSQRLHCSGHDLSEWLKGCDM
HVKIDPKIHP.

The full amino acid sequence of the disclosed NOV6b protein was found to have 75 of 147 amino acid residues (51%) identical to, and 103 of 147 amino acid residues (70%) positive with, the 147 amino acid residue protein SWISSPROT-ACC:P00705 LYSOZYME C-1 PRECURSOR (EC 3.2.1.17) (1,4-BETA-N-ACETYLMURAMIDASE C)—Anas platyrhynchos (Domestic duck) (E value=1.0e-39). Additionally, NOV 6b has similarity with the amino acid sequence of bare-faced crassow lysozyme: Expect=2.7e-39 Identities=68/129 (52%); Positives=95/129 (73%) with PIR-ID:JE0185 lysozyme (EC 3.2.1.17)—bare-faced crassow A.

High scoring similarities with human proteins include: the 148 amino acid protein SPTREMBL-ACC:O75951 LYSOZYME HOMOLOG -Expect=1.4e-33, Identities=62/141 (43%), Positives=94/141 (66%).

NOV6c

NOV6c is a novel member of the lysozyme C-1 precursor-like family of proteins, which are related to the glycoside hydrolase family. The sequence coding for the NOV6c (CG52754-02) sequence was derived by laboratory cloning of cDNA fragments, by in silico prediction of the sequence. cDNA fragments covering either the full length of the DNA sequence, or part of the sequence, or both, were cloned. In silico prediction was based on sequences available in Curagen's proprietary sequence databases or in the public human sequence databases, and provided either the full length DNA sequence, or some portion thereof.

The cDNA coding for the CG52754-02 sequence was cloned by the polymerase chain reaction (PCR) using the primers: TCTGAGGCAATGAATGGAATGAATCAC (SEQ ID NO:79) and CCAAGCCATTTACAAAATCTTTGTAAAATGC (SEQ ID NO:80). Primers were designed based on in silico predictions of the full length or some portion (one or more exons) of the cDNA/protein sequence of the invention. These primers were used to amplify a cDNA from a pool containing expressed human sequences derived from the following tissues: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea and uterus.

Multiple clones were sequenced and these fragments were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

The PCR product derived by exon linking, covering the entire open reading frame, was cloned into the pCR2.1 vector from Invitrogen to provide clone 121210::GMAC055861_A.698425.C9.

The disclosed NOV6c nucleic acid of 507 nucleotides (designated CuraGen Acc. No. CG52754-02) encoding a novel lysozyme c-I precursor-like protein is shown in Table 6H. An open reading frame was identified beginning at nucleotides 5-7 and ending at nucleotides 446-448. The start and stop codons of the open reading frame are highlighted in bold type. Putative untranslated regions (underlined), if any, are found upstream from the initiation codon and downstream from the termination codon. In a search of sequence databases, it was found, for example, that the nucleic acid sequence of this invention has 266 of 420 bases (63%) identical to a gb:GENBANK-ID:PELYSOC|acc:X60235.1 mRNA from Presbytis entellus (P.entellus mRNA for lysozyme c), E=3.5 e-16.

TABLE 6H
NOV6c Nucleotide Sequence (SEQ ID NO:19)

| | |
|---|---|
| CATAATGAAGGCCTGGGGCACTGTGGTAGTGACCTTGGCCACGCTGATGGTTGTCACTGT | (SEQ ID NO:19) |
| GGATGCCAAGATCTATGAACGCTGCGAGCTGGCGGCAAGACTGGAGAGAGCAGGGCTGAA | |
| CGGCTACAAGGGCTACGGCGTTGGAGACTGGCTGTGCATGGCTCATTATGAGAGTGGCTT | |
| TGACACCGCCTTCGTGGACCACAATCCTGATGGCAGCAGTGAATATGGCATTTTCCAACT | |
| GAATTCTGCCTGGTGGTGTGACAATGGCATTACACCCACCAAGAACCTCTGCCACATGGA | |
| TTGTCATGACCTGCTCAATCGCCATATTCTGGATGACATCAGGTGTGCCAAGCAGATTGT | |
| GTCCTCACAGAATGGGCTTTCTGCCTGGACTTCTTGGAGGCTACACTGTTCTGGCCATGA | |
| TTTATCTGAATGGCTCAAGGGGGTGTGATATGCATGTGAAAATTGATCCAAAAATTCATC | |
| CATGACTCAGATTCGAAGAGACAGATT | |

The encoded NOV6c protein having 147 amino acid residues is presented using the one-letter code in Table 61.

TABLE 6I
Encoded NOV6c protein sequence (SEQ ID NO:20).

| | |
|---|---|
| MKAWGTVVVTLATLMVVTVDA/KIYERCELAARLERAGLNGYKGYGVGDWLCMAHYESGFD | (SEQ ID NO:20) |
| TAFVDHNPDGSSEYGIFQLNSAWWCDNGITPTKNLCHMDCHDLLNRHILDDIRCAKQIVS | |
| SQNGLSAWTSWRLHCSGHDLSEWLKGV. | |

NOV6c differs from NOV6a by a C147>V amino acid change and 12 fewer amino acids at the C terminus. The full amino acid sequence of the disclosed NOV6c protein was found to have 74 of 146 amino acid residues (50%) identical to, and 102 of 146 amino acid residues (69%) similar to, the 147 amino acid residue ptnr:SWISSPROT-ACC:P00705 protein from Anas platyrhynchos (Domestic duck) (LYSOZYME C-1 PRECURSOR (EC 3.2.1.17) (1,4-BETA-N-ACETYLMURAMIDASE C)), E=5.8 e-39.

The LYSOZYME C-1 PRECURSOR-like gene disclosed in this invention is expressed in at least the following tissues: cartilage, spleen, lung, kidney, white blood cells, plasma, saliva, milk and tears. Expression information was derived from the tissue sources of the sequences that were included in the derivation of the sequence of CuraGen Acc. No. CG52754-02, NOV6c.

NOV6d

NOV6d is a novel member of the lysozyme C-1 precursor-like family of proteins, which are related to the glycoside hydrolase family. The sequence coding for the disclosed NOV6d (30412306_{—}0_{—}100_dal(B)) was derived by laboratory cloning of CDNA fragments covering the full length and/or part of the DNA sequence of the invention, and/or by in silico prediction of the full length and/or part of the DNA sequence of the invention from public human sequence databases. The laboratory cloning was performed by the method(s) summarized below:

SeqCallingTM Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human sample(s). Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

The CDNA coding for the sequence was cloned by polymerase chain reaction (PCR) using the following primers: TGACCTTGGCCACGCTGATG (SEQ ID NO: 81) and TCAATTTTCACATGCATATCACACCCC (SEQ ID NO:82) on the following pools of human cDNAs: Pool 1—Adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus. Pool2—Cancer tissue pool and Pool 3—Developmental pool.

Primers were designed based on in silico predictions for the full length or part (one or more exons) of the DNA/protein sequence of the invention or by translated homology of the predicted exons to closely related human sequences or to sequences from other species. Usually multiple clones were sequenced to derive the sequence which was then assembled similar to the SeqCalling process. In addition, sequence traces were evaluated manually and edited for corrections if appropriate.

The sequence identified by exon linking was extended in silico using information from at least some of the following sources: SeqCalling assemblies 144861150 144861122 144861127, and genomic clones gen:gb_AL356797 HTG Homo sapiens|Homo sapiens chromosome X clone RP11-404P16, 28 unordered pieces, 158749 bp splicing genomic regions: 39614-39602.

The disclosed NOV6d nucleic acid of 461 nucleotides (designated CuraGen Acc. No. 30412306_{—}0_{—}100_dal(B)) encoding a novel Lysozyme C-1 Precursor-like protein is shown in Table 6J. An open reading frame was identified beginning with an ATG initiation codon at nucleotides 1-3 and ending with a TGA codon at nucleotides 459-461. In a search of sequence databases, it was found, for example, that the nucleic acid sequence of this invention has 263 of 420 bases (62%) identical to a gb:GENBANK-ID:PELYSOC|acc:X60235 mRNA from Presbytis entellus (P.entellus mRNA for lysozyme c), E=3.2e-16.

TABLE 6J
NOV6d Nucleotide Sequence (SEQ ID NO:21)

(SEQ ID NO:21)

ATGAAGGCCTGGGGCACTGTGGTAGTGACCTTGGCCACGCTGATGGTTGT
CACTGTGGATGCCAAGATCTATGAACGCTGCGAGCTGGCGGCAAGACTGG
AGAGAGCAGGGCTGAACGGCTACAAGGGCTACGGCGTTGGAGACTGGCTG
TGCATGGCTCATTATGAGAGTGGCTTTGACACCGCCTTCGTGGACCACAA
TCCTGATGGCAGCAGTGAATATGGCATTTTCCAACTGAATTCTGCCTGGT
GGTGTGACAATGGCATTACACCCACCAAGAACCTCTGCCACATGGATTGT
CATGACCTGCTCAATCGCCATATTCTGGATGACATCAGGTGTGCCAAGCA
GATTGTGTCCTCACAGAATGGGCTTTCTGCCTGGACTTCTTGGAGGCTAC
ACTGTTCTGGCCATGATTTATCTGAATGGCTCAAGGGGTGTGATATGCAT
GTGAAAATTGA

The encoded NOV6d protein having 153 amino acid residues is presented using the one-letter code in Table 6K. NOV6d differs from NOV6a by a six amino acid deletion at the C-terminus.

TABLE 6K
Encoded NOV6d protein sequence (SEQ ID NO:22).

(SEQ ID NO:22)

MKAWGTVVVTLATLMVVTVDA/KITERCELAARLERAGLNGYKGYGVGDW
LCMAHTESGFDTAFVDHNPDGSSEYGIFQLNSAWWCDNGITPTKNLCHMD
CHDLLNRHILDDIRCAKQIVSSQNGLSAWTSWRLHCSGHDLSEWLKGCDM
HVKI.

The full amino acid sequence of the protein of the invention was found to have 75 of 147 amino acid residues (51%) identical to, and 103 of 147 amino acid residues (70%) similar to, the 147 amino acid residue ptnr:SWISSPROT-ACC:P00705 protein from Anas platyrhynchos (Domestic duck) (LYSOZYME C-1 PRECURSOR (EC 3.2.1.17) (1,4-BETA-N-ACETYLMURAMIDASE C), E=5.8 e-40.

The disclosed NOV6d Lysozyme C-1 Precursor-like protein is expressed in at least the following tissues: cartilage, spleen, lung, kidney, white blood cells, plasma, saliva, milk and tears. This information was derived by determining the tissue sources of the sequences that were included in the invention, including literature references. In addition, the sequence is predicted to be expressed in the following tissues because of the expression pattern of GENBANK-ID: gb:GENBANK-ID:PELYSOC|acc:X6023 5, a closely related P. entellus mRNA for lysozyme c homolog in species Presbytis entellus.

The NOV6 proteins of the invention show good similarity to known proteins. For example, a BLAST against patp:AAY71103, a 159 amino acid human hydrolase protein-1 (HYDRL-1) produced 152/153 (99%) identity, and 153/153 (99%) positives (E=2.6e-84). WO 00/28045. AAY71103 is described as a human hydrolase protein useful for diagnosing, treating and preventing a variety of disorders and is characterized as having homology to lysozyme-c precursor from Colobus species and Nasalis larvatis and to lysozyme from Paralichthys olivaceus. The alignment is shown in Table 6L.

TABLE 6L
Comparison of NOV6a with AAY71103.

Top Plus Strand HSPs:
Score = 852 (299.9 bits), Expect = 2.6e-84, P = 2.6e-84
Identities = 152/153 (99%), Positives = 152/153 (99%) , Frame = + 1

| | | | | |
|---|---|---|---|---|
| Query: | 1 | MKAWGTVVVTLATLMVVTVDAKIYERCELAARLERAGLNGYKGYGVGDWLCMAHYESGFD | 180 | |
| Sbjct: | 1 | MKAWGTVVVTLATLMVVTVDAKIYELCELAARLERAGLNGYKGYGVGDWLCMAHYESGFD | 60 | |
| Query: | 181 | TAFVDHNPDGSSEYGIFQLNSAWWCDNGITPTKNLCHMDCHDLLNRHILDDIRCAKQIVS | 360 | |
| Sbjct: | 61 | TAFVDHNPDGSSEYGIFQLNSAWWCDNGITPTKNLCHMDCHDLLNRHILDDIRCAKQIVS | 120 | |
| Query: | 361 | SQNGLSAWTSWRLHCSGHDLSEWLKGCDMHVKI | 459 | (SEQ ID NO:83) |
| Sbjct: | 121 | SQNGLSAWTSWRLHCSGHDLSEWLKGCDMHVKI | 153 | (SEQ ID NO:14) |

The disclosed NOV6 protein (SEQ ID NO: 14) has good identity with lysozyme-like proteins. The identity information used for ClustalW analysis is presented in Table 6M.

TABLE 6M
BLAST results for NOV6

| Gene Index/ | | Length | Identity | Positives | |
|---|---|---|---|---|---|
| Identifier | Protein/Organism | (aa) | (%) | (%) | Expect |
| gi\|14456630\|emb\|CAC | novel protein | 159 | 144/159 | 144/159 | 1e-77 |
| 41950.1\| (Z98304) | similar to | | (90%) | (90%) | |
| dJ54B20.3 | lysozyme C | | | | |
| | (1,4-beta-N- | | | | |
| | acetylmuramidase) | | | | |
| | Homo sapiens | | | | |
| gi\|126592\|sp\|P00705 | LYSOZYME C-1 | 147 | 68/127 | 95/127 | 7e-36 |
| \|LYC-1 ANAPL | PRECURSOR (1,4- | | (53%) | (74%) | |
| | BETA-N- | | | | |
| | ACETYLMURAMIDASE | | | | |
| | C) lysozyme (EC | | | | |
| | 3.2.1.17) c | | | | |
| | precursor-duck | | | | |
| gi\|547878\|sp\|P00702 | LYSOZYME C | 147 | 66/127 | 94/127 | 5e-35 |
| \|LYC PHACO | PRECURSOR (1,4- | | (51%) | (73%) | |
| | BETA-N- | | | | |
| | ACETYLMURAMIDASE | | | | |
| | C) | | | | |
| gi\|350714\|prf∥0802 | lysozyme DL1 Anas | 129 | 68/127 | 95/127 | 6e-35 |
| 160A | platyrhynchos | | (53%) | (74%) | |
| gi\|350715\|prf∥0802 | lysozyme DL3 Anas | 129 | 68/127 | 95/127 | 8e-35 |
| 160B | platyrhynchos | | (53%) | (74%) | |

This inforrnation is presented graphically in the multiple sequence alignment given in Table 6N (with NOV6a being shown on line 1) as a ClustalW analysis comparing NOV6 with related protein sequences.

[image]

[image]

The best hits from a BLASTP Non-Redundant Composite database include: the 147 amino acid protein, ptnr:SWISSPROT-ACC:P00705 LYSOZYME C-1 PRECURSOR (EC 3.2.1.17) (1,4-BETA-N-ACETYLMURAMIDASE C) from Anas platyrhynchos (Domestic duck). Expect=6.3e-40, Identities=75/147 (51%), Positives=103/147 (70%); the 129 amino acid protein ptnr:pir-id:JE0185 lysozyme (EC 3.2.1.17) from bare-faced crassow, Expect=1.7e-39, Identities=68/129 (52%), Positives=95/129 (73%); and the 147 amino acid protein ptnr:SWISSPROT-ACC:P00702 LYSOZYME C PRECURSOR (EC 3.2.1.17) (1,4-BETA-N-ACETYLMURAMIDASE C) from Phasianus colchicus (Ring-necked pheasant), Expect=5.6e-39, Identities=66/143 (46%), Positives=101/143 (70%).

Additional Blast X information for NOV6 is shown in Table 6O.

TABLE 6O
BLASTX results for NOV6

| | | | Smallest |
|---|---|---|---|
| | | | Sum |
| | Reading | High | Prob. |
| Sequences producing High-scoring Segment Pairs | Frame | Score | P (N) |
| ptnr:SWISSPROT-ACC:P00705 LYSOZYME C-1 PRECURSOR . . . | +2 | 427 | 3.8e−39 |
| ptnr:pir-id:JE0185 lysozyme (EC 3.2.1.17) - bare-faced . . . | +2 | 423 | 1.0e−38 |
| ptnr:SWISSPROT-ACC:P00702 LYSOZYME C PRECURSOR . . . | +2 | 418 | 3.4e−38 |
| ptnr:SWISSPROT-ACC:P00707 LYSOZYME C (EC 3.2.1.17) . . . | +2 | 417 | 4.3e−38 |
| ptnr:pir-id:LZFER lysozyme (EC 3.2.1.17) c precursor . . . | +2 | 413 | 1.1e−37 |
| ptnr:SWISSPROT-ACC:P00706 LYSOZYME C-3 (EC 3.2.1.17) . . . | +2 | 410 | 2.4e−37 |
| ptnr:SWISSPROT-ACC:P49663 LYSOZYME C (EC 3.2.1.17) . . . | +2 | 409 | 3.0e−37 |

The presence of identifiable domains in NOV6 was determined by searches using algorithms such as PROSITE, Blocks, Pfam, ProDomain, Prints and then determining the Interpro number by crossing the domain match (or numbers) using the Interpro website (http:www.ebi.ac.uk/interpro/).

DOMAIN results for NOV6 were collected from the Conserved Domain Database (CDD) with Reverse Position Specific BLAST. This BLAST samples domains found in the Smart and Pfam collections. The results are listed in Table 6P and Table 6Q, with the statistics and domain description. The results indicate that this protein contains the lysozyme C domain from the alpha lactalbumin family. Amino acids 22-147 NOV6a align with amino acids 1-125 of the smart00263 domain, indicated in Table 6P as SEQ ID NO:89 Amino acids 22-147 NOV6a align with amino acids 1-122 of the pfam00062 domain, indicated in Table 6Q as SEQ ID NO:90.

The good E values for NOV6a (1e-34 and 3 e-33) indicate that the sequence of NOV6 has properties similar to those of other proteins known to contain this domain.

[image]

The Interpro entry IPR001916; Lactalbmn_lysozyme (matches 173 proteins) corresponds to the Glycoside hydrolase family 22. Signature sequences include: PS00128; lactalbumin_lysozyme (149 proteins); PR00135; lyzlact (140 proteins); PF00062; lys (171 proteins); SM00263; LYZ1 (156 proteins); IPR000545; Lactalbumin (48 proteins); and IPR000974; Lysozyme (98 proteins). Glycoside hydrolase family 22 [http://afmb.cnrs-mrs.fr/˜pedro/CAZY/ghf_{—}22.html] comprises enzymes with two known activities; lysozyme type C (EC 3.2.1.17) and alpha-lactalbumins.

The domain results illustrated above also demonstrate that NOV6 contains the lysozyme C domain from the alpha lactalbumin family. Lysozyme type C and alpha-lactalbumin are similar both in terms of primary sequence and structure, and probably evolved from a common ancestral protein. Approximately 35 to 40% of the residues are conserved in both of the proteins, as well as the positions of the four disulfide bonds in each. Disulfide bonds are between Cysteine n and Cysteine n+2, e.g., the first and third cysteines. See, Prosite PDOC00119 for a diagram of the signature sequence.

Lysozyme catalyses the hydrolysis of bacterial cell wall polysaccharides; it has also been recruited for a digestive role in certain ruminants and colobine monkeys (Irwin and Wilson, J. Biol. Chem. 264:11387-11393, 1989). Another significant difference between the two enzymes is that all lactalbumins have the ability to bind calcium (Stuart et al., Nature 324:84-87, 1986), while this property is restricted to only a few lysozymes (Nitta et al., FEBS Lett., 223:405-408, 1987).

Lysozyme catalyzes the hydrolysis of certain mucopolysaccharides of bacterial cell walls. Specifically, it catalyzes the hydrolysis of the bacterial cell wall beta(1-4) glycosidic linkages between N-acetylmuramic acid and N-acetylglucosamine. It is found in spleen, lung, kidney, white blood cells, plasma, saliva, milk and tears. Fleming and Allison (Brit. J. Exp. Path.3: 252- 260, 1922) demonstrated an unusually high concentration in cartilage, indeed the highest of any tissue. Its role in cartilage is unknown. Neufeld (Personal Communication. Bethesda, Md., 1972) suggested that a genetic defect of lysozyme might underlie a skeletal dysplasia. Spitznagel et al. ((Abstract) J. Clin. Invest. 51: 93A only, 1 972) observed a patient with selective deficiency of a particular type of neutrophil granule which resulted in about 50% reduction in lysozyme levels. The patient showed increased susceptibility to infection.

Prieur et al. (Am. J. Path. 77: 283-296,1974) described inherited lysozyme deficiency in rabbits. No abnormality of cartilage or bone was noted (Greenwald et al., Biochim. Biophys. Acta 385: 435-437,1975). Older mutant rabbits showed increased susceptibility to infections, especially subcutaneous abscesses (Personal Communication. Pullman, Wash., May 13, 1975.). Camara et al. (Lab. Invest. 63: 544-550,1990) identified 2 isozymes of rabbit lysozyme and showed that their distribution was tissue specific. Leukocytic and gastrointestinal isozymes were clearly distinguished, and a possible lymphoepithelial isozyme that resembled the gastrointestinal isozyme electrophoretically and chromatographically but not kinetically was demonstrated. Mutant, lysozyme-deficient rabbits completely lacked a detectable leukocytic isozyme but had gastrointestinal and lymphoepithelial isozymes indistinguishable from those of normal rabbits. By electrophoretic methods, the mutant rabbits were shown to lack a protein band corresponding to that of the leukocytic isozyme in normal rabbits.

Yoshimura et al. (Biochem. Biophys. Res. Commun. 150:794-801,1988) isolated a cDNA encoding human lysozyme from a human placenta cDNA library. The 1.5-kb cDNA coded for a signal peptide consisting of 18 amino acids and for mature lysozyme. The amino acid sequence of the mature lysozyme, deduced from the nucleotide sequence, was identical with the published sequence. Human lysozyme has 130 amino acid residues and 4 disulfide bonds (Taniyama et al., J. Biol. Chem. 266: 6456-6461, 1991). Peters et al. ((Abstract) Cytogenet. Cell Genet. 51: 1059 only,1989) described the isolation of 2 overlapping genomic clones containing 25 kb of the human lysozyme gene region. They also isolated a full-length human lysozyme cDNA clone from a human placental cDNA library. They reported on the nucleotide sequence of the entire structural gene and the cDNA clone. Using a panel of somatic cell hybrids, Peters et al. (Biochemistry 38: 6419-6427,1989) assigned the lysozyme gene to human chromosome 12.

Canet et al. (1999) studied the unfolding and refolding properties of human lysozyme and 2 of its amyloidogenic variants, ile56 to thr and asp67 to his, by stopped-flow fluorescence and hydrogen exchange pulse labeling coupled with mass spectrometry. Their results suggested that the amyloidogenic nature of the lysozyme variants arises from a decrease in the stability of the native fold relative to partially folded intermediates. The origin of this instability was different in the 2 variants, being caused in one case primarily by a reduction in the folding rate and in the other by an increase in the unfolding rate. In both cases, this resulted in a low population of soluble partially folded species that can aggregate in a slow and controlled manner to form amyloid fibrils.

In the human, mutations in the LYZ gene in renal amyloidosis represented the first link of lysozyme to genetic disease (see OMIM 153450, 153450.0001 and 153450.0002). See generally, Prosite PDC00119, Interpro entries IPR001916, IPR000974 and IPR000545.

The disclosed NOV6 lysozyme C-1 precursor-like protein maps to chromosome Xp11.1-11.3. This information was assigned using OMIM, the electronic northern bioinformatic tool implemented by CuraGen Corporation, public ESTs, public literature references and/or genomic clone homologies. This was executed to derive the chromosomal mapping of the SeqCalling assemblies, Genomic clones, literature references and/or EST sequences that were included in the invention.

The disclosed NOV6a lysozyme C-1 precursor-like protein is expressed in at least the following tissues: cartilage, testis, fetal brain, spleen, lung, kidney, white blood cells, plasma, saliva, milk, tears. This information was derived by determining the tissue sources of the sequences that were included in the invention including but not limited to SeqCalling sources, Public EST sources, Genomic Clone sources, Literature sources, and/or RACE sources.

The protein similarity information, expression pattern, and map location for the lysozyme C-1 precursor-like protein and nucleic acid disclosed herein suggest that this lysozyme C-1 precursor may have important structural and/or physiological functions characteristic of the lysozyme C-1 precursor family, and the related glycoside hydrolase family. Therefore, the novel nucleic acids and NOV6 proteins of the invention, or fragments thereof, are useful in potential diagnostic and therapeutic applications and as a research tool. These include serving as a specific or selective nucleic acid or protein diagnostic and/or prognostic marker, wherein the presence or amount of the nucleic acid or the protein are to be assessed, swell as potential therapeutic applications such as the following: (i)a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The NOV6 nucleic acids and proteins of the invention are useful in potential diagnostic and therapeutic applications implicated in various diseases and disorders described below and/or other pathologies. For example, the compositions of the present invention will have efficacy for treatment of patients suffering from: susceptibility to infection, amyloidosis; blood disorders including hemophilia and hypercoagulation; salivitory disorders, digestive disorders, inflammatory processes, muscle, bone and tendon disorders; idiopathic thrombocytopenic purpura; immunodeficiencies; graft versus host; infection; systemic lupus erythematosus; autoimmune disease; asthma, emphysema; scleroderma; allergy; ARDS; diabetes; renal artery stenosis; interstitial nephritis; glomerulonephritis; polycystic kidney disease; Renal tubular acidosis; IgA nephropathy; hypercalceimia; Lesch-Nyhan syndrome; renal amyloidosis; arthritis; tendonitis; reproductive disorders, chorioathetosis with mental retardation and abnormal behavior; renal cell carcinoma, papillary, 2; renpenning syndrome-1; sarcoma, synovial; arthrogryposis, X-linked (spinal muscular atrophy, infantile, X-linked) mental retardation syndrome; X-linked, siderius type mental retardation; X-linked 14 mental retardation; X-linked nonspecific, type 50 mental retardation; X-linked, syndromic 7 mental retardation; retinitis pigmentosa-2; Von Hippel-Lindau (VHL) syndrome, Alzheimer's disease, stroke, tuberous sclerosis, hypercalceimia, Parkinson's disease, Huntington's disease, cerebral palsy, epilepsy, multiple sclerosis, ataxia-telangiectasia, leukodystrophies, behavioral disorders, addiction, anxiety, pain and other diseases, disorders and conditions of the like.

The polypeptides can be used as immunogens to produce antibodies specific for the invention, and as vaccines. They can also be used to screen for potential agonist and antagonist compounds. For example, a cDNA encoding the lysozyme C-like protein may be useful in gene therapy, and the Lysozyme C-like protein may be useful when administered to a subject in need thereof. These materials are further useful in the generation of antibodies that bind immuno-specifically to the novel NOV6 substances for use in therapeutic or diagnostic methods. These antibodies may be generated according to methods known in the art, using prediction from hydrophobicity charts, as described in the “Anti-NOVX Antibodies” section below.

NOV6b has been analyzed for tissue expression profiles as described in Example 1 below.

NOV7

NOV7 includes a family of three similar nucleic acids and three similar proteins, designated NOV7a, NOV7b, and NOV7c, disclosed below. The disclosed nucleic acids encode proteins belonging to the immunoglobulin superfamily.

NOV7a

The disclosed NOV7a nucleic acid of 3430 nucleotides (also referred to as CG5 373-10) is shown in Table 7A. An ORF begins with an ATG initiation codon at nucleotides 351-353 and ends with a TGA codon at nucleotides 2490-2492. A putative untranslated region upstream from the initiation codon and downstream from the termination codon is underlined in Table 7A, and the start and stop codons are in bold letters. The NOV7a gene maps to chromosome 21q21. This assignment was made using mapping information associated with genomic clones, public genes and ESTs sharing sequence identity with the disclosed sequence and CuraGen Corporation's Electronic Northern bioinformatic tool.

TABLE 7A
NOV7a Nucleotide Sequence (SEQ ID NO:23)

CTCTCCGATACTTTCTCCCAAGGGTCAGCTGCTTCTTCATTCCAAGTGGA
CAAGGAGCCAGCTGCTCACTGTCCTTGAGAGACTTCAGCGAGAGACCAGG
GTGTCCAGGCTCCATGCAGGAAAGCCATGCGTATAAATTCCACCTCTGAG
CCAGGCCTCACCAGCAAGCCCACTCTTAAGCCCTTGACTTGGGCTCCAGG
GGCCATGGGAAGGAGAAACGGACCCAGACCCGCTTCAGCCAGGAGCCAGC
TGACCAGACGGTGGTGGCTGGACAGCGGGCCGTGCTCCCCTGTGTGCTGC
TCAACTACTCTGGAATTGTGCAATGGACCAAGGACGGGCTGGCCCTGGGC
ATGGGCCAGGCCCTCAAAGCCTGGCCACGGTACCGGGTTGTGGGCTCCGC
AGACGCTGGGCAGTACAACCTGGAGATCACAGATGCTGAGCTCTCTGACG
ACGCCTCTTACGAGTGCCAGGCCACGGAGGCCGCCCTGCGCTCTCGGCGG
GCCAAACTCACCGTGCTCATCCCCCCAGAGGACACCAGGATTGACGGAGG
CCCTGTGATTCTACTGCAGGCAGGCACCCCCCACAACCTCACATGCCGGG
CCTTCAATGCGAAGCCTGCTGCCACCATCATCTGGTTCCGGGACGGGACG
CAGCAGGAGGGCGCTGTGGCCAGCACGGAATTGCTGAAGGATGGGAAGAG
GGAGACCACCGTGAGCCAACTGCTTATTAACCCCACGGACCTGGACATAG
GGCGTGTCTTCACTTGCCGAAGCATGAACGAAGCCATCCCTAGTGGCAAG
GAGACTTCCATCGAGCTGGATGTGCACCACCCTCCTACAGTGACCCTGTC
CATTGAGCCACAGACGGTGCAGGAGGGTGAGCGTGTTGTCTTTACCTGCC
AGGCCACAGCCAACCCCGAGATCTTGGGCTACAGGTGGGCCAAAGGGGGT
TTCTTGATTGAAGACGCCCACGAGAGTCGCTATGAGACAAATGTGGATTA
TTCCTTTTTCACGGAGCCTGTGTCTTGTGAGGTTCACAACAAAGTGGGAA
GCACCAATGTCAGCACTTTAGTAAATGTCCACTTTGCTCCCCGGATTGTA
GTTGACCCCAAACCCACAACCACAGACATTGGCTCTGATGTGACCCTTAC
CTGTGTCTGGGTTGGGGAAATCCCCCCCTCACTCTCACCTGGACCAAAAA
GGACTCAAATATTGGGGCCCTGGCTTCTTGGTTCCCCACCCGAGGCTGCT
CTCTCTGCCCAGGTCCTGAGTAACAGCAACCAGCTGCTGCTGAAGTCGGT
GACTCAGGCAGACGCTGGCACCTACACCTGCCGGGCCATCGTGCCTCGAA
TCGGAGTGGCTGAGCGGGAGGTGCCGCTCTATGTGAACGGGCCCCCCATC
ATCTCCAGTGAGGCAGTGCAGTATGCTGTGAGGGGTGACGGTGGCAAGGT
GGAGTGTTTCATTGGGAGCACACCACCCCCAGACCGCATAGCATGGGCCT
GGAAGGAGAACTTCTTGGAGGTGGGGACCCTGGAACGCTATACAGTGGAG
AGGACCAACTCAGGCAGTGGGGTGCTATCCACGCTCACCATCAACAATGT
CATGGAGGCCGACTTTCAGACTCACTACAACTGCACCGCCTGGAACAGCT
TCGGGCCAGGCACAGCCATCATCCAGCTGGAAGAGCGAGAGGTGTTACCT
GTGGGCATCATAGCTGGGGCCACCATCGGCGCGAGCATCCTGCTCATCTT
CTTCTTCATCGCCTTGGTATTCTTCCTCTACCGGCGCCGCAAAGGCAGTC
GCAAAGACGTGACCCTGAGGAAGCTGGATATCAAGGTGGAGACAGTGAAC
CGAGAGCCACTTACGATGCATTCTGACCGGGAGGATGACACCGCCAGCGT
CTCCACAGCAACCCGGGTCATGAAGGCCATCTACTCGTCGTTTAAGGATG
ATGTGGATCTGAAGCAGGACCTGCGCTGCGACACCATCGACACCCGGGAG
GAGTATGAGATGAAGGACCCCACCAATGGCTACTACAACGTGCGTGCCCA
TGAAGACCGCCCGTCTTCCAGGGCAGTGCTCTATGCTGACTACCGTGCCC
CTGGCCCTGCCCGCTTCGACGGCCGCCCCTCATCCCGTCTCTCCCACTCC
AGCGGCTATGCCCAGCTCAACACCTATAGCCGGGGCCCTGCCTCTGACTA
TGGCCCTGAGCCCACACCCCCTGGCCCTGCTGCCCCAGCTGGCACTGACA
CAACCAGCCAGCTGTCCTACGAGAACTATGAGAAGTTCAACTCCCATCCC
TTCCCTGGGGCAGCTGGGTACCCCACCTACCGACTGGGCTACCCCCAGGC
CCCACCCTCTGGCCTGGAGCGGACCCCATATGAGGCGTATGACCCCATTG
GCAAGTACGCCACAGCCACTCGATTCTCCTACACCTCCCAGCACTCGGAC
TACGGCCAGCGATTCCAGCAGCGCATGCAGACTCACGTGTAGGGGCCAGA
GCCTGGCTGGGGCATCTCTGCGGGGCAGAGGAGAAGGCTTTCGCAGCTGT
TCCCTGATATTCAGGGACATTGCTCATTGCTCCCTTCTCGGACCAGCCTT
CTTCCTCCCACCATGGCAGGTGGGGAGCAGGTCTCCCAGAGACACCCCGT
CCCGAGGATGGTGCTCTGTGCATGCCCCAGCCTCCTGGGCCTGCCCTTCC
CTCTTCTTCGGGAGGATGTGTCTCTTCTGACCTGCACTCTTGCCTGACCC
TAGAATGGGGACAGGGAAAGTGAAGGTTAGGGAAAGCAGAGGGGGGCACT
TTTTAGCATTCCCTTTCTATCCCACCCCTCTGATCTCCCATAAGTGGAAA
TGGGGGTACCCAGGGATGGGCAGGCTTTGGCCTAGGGACATGAAGTATGG
GAGTGGGTGGCTGTGGCACAGACAGGTGGAAAACGGGATAGCCTGGCCAG
TCCCTCTGTTGTCTGCATTCGTGCCCTGGGTGCCTCTCTCCTTCCTCAGG
GTACTGCAGAAGGGAGCGAACAGGGTACTGTTCGCTCTTGTCTACAGAAC
AGCCCTGGCACTGCATTCAAATCCAGTCTTCATTCAGCTGGGATCAAAAT
GCCAGTCACCTTGGCTACCCACTGTGGACAGCTGTCTGTCAGCATGCAGA
GGGATCCAGGAATCCCCCCGGCAGCACGGCCCGCTTTCCTTCTCCTCCAT
GCTGGGCCAGCCAGATAAGTCAGGGTCCTGGTGGAGAAAGAAAGGCTAGG
ACCATGTCCTCATTGACCCAGATACTGCTGTGTGCTGCACAGCAGTGAAC
CAACACTAGAGGGAGCCACACAAGCCTCCTCTCCCCAGTCTGCCCCACTT
CCTGGCTTTAACTCTTGAGCTGGTTTGGGGAGTGGTGAGGTAGGGGTGGG
GGTGCTGTAGGCTCTTTTTCAAAAAAAAAC

The NOV7a protein encoded by SEQ ID NO:24 has 713 amino acid residues and is presented using the one-letter code in Table 7B. The Psort profile for NOV7a predicts that this sequence is likely to be localized at the plasma membrane with a certainty of 0.7000.

TABLE 7B
Encoded NOV7a protein sequence (SEQ ID NO:24)

MGQALKAWPRYRVVGSADAGQYNLEITDAELSDDASYECQATEAALRSRR
AKLTVLIPPEDTRIDGGPVILLQAGTPHNLTCRAFNAKPAATIIWFRDGT
QQEGAVASTELLKDGKRETTVSQLLINPTDLDIGRVFTCRSMNEAIPSGK
ETSIELDVHHPPTVTLSIEPQTVQEGERVVFTCQATANPEILGYRWAKGG
FLIEDAHESRYETNVDYSFFTEPVSCEVHNKVGSTNVSTLVNVHFAPRIV
VDPKPTTTDIGSDVTLTCVWVGEIPPSLSPGPKRTQILGPWLLGSPPEAA
LSAQVLSNSNQLLLKSVTQADAGTYTCRAIVPRIGVAEREVPLYVNGPPI
ISSEAVQYAVRGDGGKVECFIGSTPPPDRIAWAWKENFLEVGTLERYTVE
RTNSGSGVLSTLTINNVMEADFQTHYNCTAWNSFGPGTAIIQLEEREVLP
VGIIAGATIGASILLIFFFIALVFFLYRRRKGSRKDVTLRKLDIKVETVN
REPLTMHSDREDDTASVSTATRVMKAIYSSFKDDVDLKQDLRCDTIDTRE
EYEMKDPTNGYYNVRAHEDRPSSRAVLYADYRAPGPARFDGRPSSRLSHS
SGYAQLNTYSRGPASDYGPEPTPPGPAAPAGTDTTSQLSYENYEKFNSHP
FPGAAGYPTYRLGYPQAPPSGLERTPYEAYDPIGKYATATRFSYTSQHSD
YGQRFQQRMQTHV

The disclosed nucleic acid sequence for NOV7a has 2649 of 2659 bases (99%) identical to Homo sapiens cDNA FLJ12646 fis, clone NT2RM4001987 weakly similar to Neural cell adhesion molecule 1, large isoform precursor (GENBANK-ID:AK022708|acc:AK022708.1) Similarly, the full NOV7a amino acid sequence was found to have 556 of 572 amino acid residues (97%) identical to, and 558 of 572 amino acid residues (97%) similar to, the 571 amino acid residue to Homo sapiens cDNA FLJ12646 FIS, Clone NT2RM4001987 weakly similar to Neural cell adhesion molecule 1, large isoform precursor (SPTREMBL-ACC:Q9H9N1). Cell adhesion molecules are a subset of the immunoglobulin superfamily of proteins.

The presence of identifiable domains in the protein disclosed herein was determined by searches using domain databases such as Pfam, PROSITE, ProDom, Blocks or Prints and then identified by the Interpro domain accession number. Significant domains are summarized in Table 7C.

TABLE 7C
DOMAIN results for NOV7a
Scores for sequence family classification (score includes all domains)

| Model | Description | Score | E-value | N |
|---|---|---|---|---|
| ig | Immunoglobulin domain | 56.6 | 5.3e-16 | 4 |
| PKD | PKD domain | −3.5 | 2.6 | 1 |
| Adeno_E3_CR2 | Adenovirus E3 region protein | −4.6 | 8.4 | 1 |
| | CR2 | | | |

Based on its relatedness to the neural cell adhesion molecule and the presence of immunoglobulin-like domains, the NOV7a protein is a novel cell adhesion molecule belonging to the immunoglobulin superfamily of proteins.

Possible cSNPs for NOV 7a include those found in Table 7D.

TABLE 7D
cSNPs for NOV7a

| Base Position of | | | Amino Acid |
|---|---|---|---|
| cSNP | Wild Type | Variant | Change |
| 2917 | C | A | none |
| 1945 | A | I | Lys to Met |
| 928 | C | A | Gly to Asp |
| 917 | C | G | none |
| 655 | A | G | Gln to Arg |
| 472 | C | I | Ala to Val |
| 659 | C | A | none |
| 713 | G | A | none |
| 767 | C | T | none |

The NOV7a gene is expressed in adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea and uterus. Accordingly, a NOV7a nucleic acid is useful in identifying these tissue types.

NOV7b

The disclosed NOV7b nucleic acid of 3379 nucleotides (also referred to as 20421338_{—}1 or CG51373-03) is shown in Table 7E. An ORF begins with an ATG initiation codon at nucleotides 351-353 and ends with a TGA codon at nucleotides 2439-2441.

TABLE 7E
NOV7b Nucleotide Sequence (SEQ ID NO:25)

| | |
|---|---|
| 1 | CTCTCCGATACTTTCTCCCAAGGGTCAGCTGCTTCTTCATTCCAA |

GTGGACAAGGAGCCAGCTGCTCACTGTCCTTGAGA

| | |
|---|---|
| 81 | GACTTCAGCGAGAGACCAGGGTGTCCAGGCTCCATGCAGGAAAGC |

CATGCGTATAAATTCCACCTCTGAGCCAGGCCTCA

| | |
|---|---|
| 161 | CCAGCAAGCCCACTCTTAAGCCCTTGACTTGGGCTCCAGGGGCCA |

TGGGAAGGAGAAACGGACCCAGACCCGCTTCAGCC

| | |
|---|---|
| 241 | AGGAGCCAGCTGACCAGACGGTGGTGGCTGGACAGCGGGCCGTGC |

TCCCCTGTGTGCTGCTCAACTACTCTGGAATTGTG

| | |
|---|---|
| 321 | CAATGGACCAAGGACGGGCTGGCCCTGGGCATGGGCCAGGCCCTC |

AAAGCCTGGCCACGGTACCGGGTTGTGGGCTCCGC

| | |
|---|---|
| 401 | AGACGCTGGGCAGTACAACCTGGAGATCACAGATGCTGAGCTCTC |

TGACGACGCCTCTTACGAGTGCCAGGCCACGGAGG

| | |
|---|---|
| 481 | CCGCCCTGCGCTCTCGGCGGGCCAAACTCACCGTGCTCATCCCCC |

CAGAGGACACCAGGATTGACGGAGGCCCTGTGATT

| | |
|---|---|
| 561 | CTACTGCAGGCAGGCACCCCCCACAACCTCACATGCCGGGCCTTC |

AATGCGAAGCCTGCTGCCACCATCATCTGGTTCCG

| | |
|---|---|
| 641 | GGACGGGACGCAGCAGGAGGGCGCTGTGGCCAGCACGGAATTGCT |

GAAGGATGGGAAGAGGGAGACCACCGTGAGCCAAC

| | |
|---|---|
| 721 | TGCTTATTAACCCCACGGACCTGGACATAGGGCGTGTCTTCACTT |

GCCGAAGCATGAACGAAGCCATCCCTAGTGGCAAG

| | |
|---|---|
| 801 | GAGACTTCCATCGAGCTGGATGTGCACCACCCTCCTACAGTGACC |

CTGTCCATTGAGCCACAGACGGTGCAGGAGGGTGA

| | |
|---|---|
| 881 | GCGTGTTGTCTTTACCTGCCAGGCCACAGCCAACCCCGAGATCTT |

GGGCTACAGGTGGGCCAAAGGGGGTTTCTTGATTG

| | |
|---|---|
| 961 | AAGACGCCCACGAGAGTCGCTATGAGACAAATGTGGATTATTCCT |

TTTTCACGGAGCCTGTGTCTTGTGAGGTTCACAAC

| | |
|---|---|
| 1041 | AAAGTGGGAAGCACCAATGTCAGCACTTTAGTAAATGTCCACTTT |

GCTCCCCGGATTGTAGTTGACCCCAAACCCACAAC

| | |
|---|---|
| 1121 | CACAGACATTGGCTCTGATGTGACCCTTACCTGTGTCTGGGTTGG |

GAATCCCCCCCTCACTCTCACCTGGACCAAAAAGG

| | |
|---|---|
| 1201 | ACTCAAATATGGTCCTGAGTAACAGCAACCAGCTGCTGCTGAAGT |

CGGTGACTCAGGCAGACGCTGGCACCTACACCTGC

| | |
|---|---|
| 1281 | CGGGCCATCGTGCCTCGAATCGGAGTGGCTGAGCGGGAGGTGCCG |

CTCTATGTGAACGGGCCCCCCATCATCTCCAGTGA

| | |
|---|---|
| 1361 | GGCAGTGCAGTATGCTGTGAGGGGTGACGGTGGCAAGGTGGAGTG |

TTTCATTGGGAGCACACCACCCCCAGACCGCATAG

| | |
|---|---|
| 1441 | CATGGGCCTGGAAGGAGAACTTCTTGGAGGTGGGGACCCTGGAAC |

GCTATACAGTGGAGAGGACCAACTCAGGCAGTGGG

| | |
|---|---|
| 1521 | GTGCTATCCACGCTCACCATCAACAATGTCATGGAGGCCGACTTT |

CAGACTCACTACAACTGCACCGCCTGGAACAGCTT

| | |
|---|---|
| 1601 | CGGGCCAGGCACAGCCATCATCCAGCTGGAAGAGCGAGAGGTGTT |

ACCTGTGGGCATCATAGCTGGGGCCACCATCGGCG

| | |
|---|---|
| 1681 | CGAGCATCCTGCTCATCTTCTTCTTCATCGCCTTGGTATTCTTCC |

TCTACCGGCGCCGCAAAGGCAGTCGCAAAGACGTG

| | |
|---|---|
| 1761 | ACCCTGAGGAAGCTGGATATCAAGGTGGAGACAGTGAACCGAGAG |

CCACTTACGATGCATTCTGACCGGGAGGATGACAC

| | |
|---|---|
| 1841 | CGCCAGCGTCTCCACAGCAACCCGGGTCATGAAGGCCATCTACTC |

GTCGTTTAAGGATGATGTGGATCTGAAGCAGGACC

| | |
|---|---|
| 1921 | TGCGCTGCGACACCATCGACACCCGGGAGGAGTATGAGATGAAGG |

ACCCCACCAATGGCTACTACAACGTGCGTGCCCAT

| | |
|---|---|
| 2001 | GAAGACCGCCCGTCTTCCAGGGCAGTGCTCTATGCTGACTACCGT |

GCCCCTGGCCCTGCCCGCTTCGACGGCCGCCCCTC

| | |
|---|---|
| 2081 | ATCCCGTCTCTCCCACTCCAGCGGCTATGCCCAGCTCAACACCTA |

TAGCCGGGGCCCTGCCTCTGACTATGGCCCTGAGC

| | |
|---|---|
| 2161 | CCACACCCCCTGGCCCTGCTGCCCCAGCTGGCACTGACACAACCA |

GCCAGCTGTCCTACGAGAACTATGAGAAGTTCAAC

| | |
|---|---|
| 2241 | TCCCATCCCTTCCCTGGGGCAGCTGGGTACCCCACCTACCGACTG |

GGCTACCCCCAGGCCCCACCCTCTGGCCTGGAGCG

| | |
|---|---|
| 2321 | GACCCCATATGAGGCGTATGACCCCATTGGCAAGTACGCCACAGC |

CACTCGATTCTCCTACACCTCCCAGCACTCGGACT

| | |
|---|---|
| 2401 | ACGGCCAGCGATTCCAGCAGCGCATGCAGACTCACGTGTAGGGGC |

CAGAGCCTGGCTGGGGCATCTCTGCGGGGCAGAGG

| | |
|---|---|
| 2481 | AGAAGGCTTTCGCAGCTGTTCCCTGATATTCAGGGACATTGCTCA |

TTGCTCCCTTCTCGGACCAGCCTTCTTCCTCCCAC

| | |
|---|---|
| 2561 | CATGGCAGGTGGGGAGCAGGTCTCCCAGAGACACCCCGTCCCGAG |

GATGGTGCTCTGTGCATGCCCCAGCCTCCTGGGCC

| | |
|---|---|
| 2641 | TGCCCTTCCCTCTTCTTCGGGAGGATGTGTCTCTTCTGACCTGCA |

CTCTTGCCTGACCCTAGAATGGGGACAGGGAAAGT

| | |
|---|---|
| 2721 | GAAGGTTAGGGAAAGCAGAGGGGGGCACTTTTTAGCATTCCCTTT |

CTATCCCACCCCTCTGATCTCCCATAAGTGGAAAT

| | |
|---|---|
| 2801 | GGGGGTACCCAGGGATGGGCAGGCTTTGGCCTAGGGACATGAAGT |

ATGGGAGTGGGTGGCTGTGGCACAGACAGGTGGAA

| | |
|---|---|
| 2881 | AACGGGATAGCCTGGCCAGTCCCTCTGTTGTCTGCATTCGTGCCC |

TGGGTGCCTCTCTCCTTCCTCAGGGTACTGCAGAA

| | |
|---|---|
| 2961 | GGGAGCGAACAGGGTACTGTTCGCTCTTGTCTACAGAACAGCCCT |

GGCACTGCATTCAAATCCAGTCTTCATTCAGCTGG

| | |
|---|---|
| 3041 | GATCAAAATGCCAGTCACCTTGGCTACCCACTGTGGACAGCTGTC |

TGTCAGCATGCAGAGGGATCCAGGAATCCCCCCGG

| | |
|---|---|
| 3121 | CAGCACGGCCCGCTTTCCTTCTCCTCCATGCTGGGCCAGCCAGAT |

AAGTCAGGGTCCTGGTGGAGAAAGAAAGGCTAGGA

| | |
|---|---|
| 3201 | CCATGTCCTCATTGACCCAGATACTGCTGTGTGCTGCACAGCAGT |

GAACCAACACTAGAGGGAGCCACACAAGCCTCCTC

| | |
|---|---|
| 3281 | TCCCCAGTCTGCCCCACTTCCTGGCTTTAACTCTTGAGCTGGTTT |

GGGGAGTGGTGAGGTAGGGGTGGGGGTGCTGTAGG

| | |
|---|---|
| 3361 | CTCTTTTTCAAAAAAAAAC |

The NOV7b gene is expressed in lymph node, ovary, fetal lung, fetal kidney and adrenal gland. Accordingly, a NOV7b nucleic acid is useful in identifying these tissue types.

The encoded NOV7b protein is presented in Table 7F. The disclosed protein is 696 amino acids long and is denoted by SEQ ID NO:26. Like NOV7a, the Psort profile for NOV7b predicts that this is likely to be localized at the plasma membrane with a certainty of 0.7000.

Table 7F
Encoded NOV7b protein sequence (SEQ ID NO:26)

| | |
|---|---|
| 1 | MGQALKAWPRYRVVGSADAGQYNLEITDAELSDDASYECQATEAALR |

SRRAKLTVLIPPEDTRIDGGPVILLQAGTPHNL

| | |
|---|---|
| 81 | TCRAFNAKPAATIIWFRDGTQQEGAVASTELLKDGKRETTVSQLLIN |

PTDLDIGRVFTCRSMNEAIPSGKETSIELDVHH

| | |
|---|---|
| 161 | PPTVTLSIEPQTVQEGERVVFTCQATANPEILGYRWAKGGFLIEDAH |

ESRYETNVDYSFFTEPVSCEVHNKVGSTNVSTL

| | |
|---|---|
| 241 | VNVHFAPRIVVDPKPTTTDIGSDVTLTCVWVGNPPLTLTWTKKDSNM |

VLSNSNQLLLKSVTQADAGTYTCRAIVPRIGVA

| | |
|---|---|
| 321 | EREVPLYVNGPPIISSEAVQYAVRGDGGKVECFIGSTPPPDRIAWAW |

KENFLEVGTLERYTVERTNSGSGVLSTLTINNV

| | |
|---|---|
| 401 | MEADFQTHYNCTAWNSFGPGTAIIQLEEREVLPVGIIAGATIGASIL |

LIFFFIALVFFLYRRRKGSRKDVTLRKLDIKVE

| | |
|---|---|
| 481 | TVNREPLTMHSDREDDTASVSTATRVMKAIYSSFKDDVDLKQDLRCD |

TIDTREEYEMKDPTNGYYNVRAHEDRPSSRAVL

| | |
|---|---|
| 561 | YADYRAPGPARFDGRPSSRLSHSSGYAQLNTYSRGPASDYGPEPTPP |

GPAAPAGTDTTSQLSYENYEKFNSHPFPGAAGY

| | |
|---|---|
| 641 | PTYRLGYPQAPPSGLERTPYEAYDPIGKYATATRFSYTSQHSDYGQR |

FQQRMQTHV

In a search of sequence databases, it was found, for example, that the NOV7b polypeptide sequence has 410 of 410 amino acids (100%) identical to irregular chiasm c-roughest protein precursor from homo sapiens (GenBank Accession No. BAA91850).

Possible cSNPs for NOV 7b include those found in Table 7G.

TABLE 7G
cSNPs for NOV7b

| Base Position of | | | Amino Acid |
|---|---|---|---|
| cSNP | Wild Type | Variant | Change |
| 2866 | C | A | none |
| 1894 | A | T | Lys to Met |
| 928 | G | A | Gly to Asp |
| 917 | C | G | none |
| 655 | A | G | Gln to Arg |
| 472 | C | T | Ala to Val |
| 659 | G | A | none |
| 713 | 6 | A | none |
| 767 | C | T | none |

NOV7c

The disclosed NOV7c nucleic acid of 1145 nucleotides (also referred to as 2041338_{—}2 or CG51373-04) is shown in Table 7H. An ORF begins with an ATG initiation codon at nucleotides 351-353 and ends with a TGA codon at nucleotides 918-920.

TABLE 7H
NOV7c Nucleotide Sequence (SEQ ID NO:27)

| | |
|---|---|
| 1 | CTCTCCGATACTTTCTCCCAAGGGTCAGCTGCTTCTTCATTCCAA |

GTGGACAAGGAGCCAGCTGCTCACTGTCCTTGAGA

| | |
|---|---|
| 81 | GACTTCAGCGAGAGACCAGGGTGTCCAGGCTCCATGCAGGAAAGC |

CATGCGTATAAATTCCACCTCTGAGCCAGGCCTCA

| | |
|---|---|
| 161 | CCAGCAAGCCCACTCTTAAGCCCTTGACTTGGGCTCCAGGGGCCA |

TGGGAAGGAGAAACGGACCCAGACCCGCTTCAGCC

| | |
|---|---|
| 241 | AGGAGCCAGCTGACCAGACGGTGGTGGCTGGACAGCGGGCCGTGC |

TCCCCTGTGTGCTGCTCAACTACTCTGGAATTGTG

| | |
|---|---|
| 321 | CAATGGACCAAGGACGGGCTGGCCCTGGGCATGGGCCAGGCCCTC |

AAAGCCTGGCCACGGTACCGGGTTGTGGGCTCCGC

| | |
|---|---|
| 401 | AGACGCTGGGCAGTACAACCTGGAGATCACAGATGCTGAGCTCTC |

TGACGACGCCTCTTACGAGTGCCAGGCCACGGAGG

| | |
|---|---|
| 481 | CCGCCCTGCGCTCTCGGCGGGCCAAACTCACCGTGCTCATCCCCC |

CAGAGGACACCAGGATTGACGGAGGCCCTGTGATT

| | |
|---|---|
| 561 | CTACTGCAGGCAGGCACCCCCCACAACCTCACATGCCGGGCCTTC |

AATGCGAAGCCTGCTGCCACCATCATCTGGTTCCG

| | |
|---|---|
| 641 | GGACGGGACGCAGCAGGAGGGCGCTGTGGCCAGCACGGAATTGCT |

GAAGGATGGGAAGAGGGAGACCACCGTGAGCCAAC

| | |
|---|---|
| 721 | TGCTTATTAACCCCACGGACCTGGACATAGGGCGTGTCTTCACTT |

GCCGAAGCATGAACGAAGCCATCCCTAGTGGCAAG

| | |
|---|---|
| 801 | GAGACTTCCATCGAGCTGGATGTGCACCGTGAGTGGGCTGGGGGG |

AGCAGTCTGGAGCAGGGGGGTGGAAGAAGGGGTGT

| | |
|---|---|
| 881 | GTTTGAGAAGCACACTCTTAGTTTGAGAAACACAAACTAAGAGTC |

CCCCTATGGTCCCCAGGACAAACGCTTGCCTTCTT

| | |
|---|---|
| 961 | CACATCTTTCATTCCCTGGATTGAACCATGGGGACTAAGGGCTGG |

TAGAGCATTGGCTGTGGAGTCAGGCAGTCCCCAGG

| | |
|---|---|
| 1041 | TCTAAACCAGCCTGTTATTAGTCAATGGTTTACACTCTCTGGGCC |

TCGGTTTCCAGTTCTGTATACTGTATATTGCAAAA

| | |
|---|---|
| 1121 | GATAAAATACTGGCCTACAGCCCCA |

The NOV7c gene is expressed in lymph node, ovary and adrenal gland. Accordingly, a NOV7c nucleic acid is useful in identifying these tissue types.

The NOV7c protein encoded by SEQ ID NO:28 has 189 amino acid residues and is presented using the one-letter code in Table 7I. The Psort profile for NOV7c predicts that this is likely to be localized at the cytoplasm with a certainty of 0.4500.

TABLE 7I
Encoded NOV7c protein sequence (SEQ ID NO:28)

| | |
|---|---|
| 1 | MGQALKAWPRYRVVGSADAGQYNLEITDAELSDDASYECQATEAALR |

SRRAKLTVLIPPEDTRIDGGPVILLQAGTPHNL

| | |
|---|---|
| 81 | TCRAFNAKPAATIIWFRDGTQQEGAVASTELLKDGKRETTVSQLLINP |

TDLDIGRVFTCRSMNEAIPSGKETSIELDVHR

| | |
|---|---|
| 161 | EWAGGSSLEQGGGRRGVFEKHTLSLRNTN |

In a search of sequence databases, it was found, for example, that the NOV7c polypeptide sequence has 45 of 135 amino acids (33%) identical to F22162_{—}1 from homo sapiens (GenBank Accession No. Q9Y4A4).

Possible cSNPs for NOV 7c include those found in Table 7J.

TABLE 7J
cSNPs for NOV7c

| Base Position of | | | Amino Acid |
|---|---|---|---|
| cSNP | Wild Type | Variant | Change |
| 655 | A | 6 | Gln to Arg |
| 472 | C | I | Ala to Val |
| 659 | G | A | none |
| 713 | G | A | none |
| 767 | C | T | none |
| 1071 | 1 | A | none |
| 1076 | T | C | none |

Unless specifically addressed as NOV7a, NOV7b or NOV7c, any reference to NOV7 is assumed to encompass all variants. Residue differences between any NOVX variant sequences herein are written to show the residue in the “a” variant and the residue position with respect to the “a”varient. NOV7 residues in all following sequence alignments that differ between the individual NOV7 variants are highlighted with a box and marked with the (o) symbol above the variant residue in all alignments herein. For example, the protein shown in line 1 of Table 7L depicts the sequence for NOV7a, and the positions where NOV7b differs are marked with a (o) symbol and are highlighted with a box.

The disclosed NOV7 protein has good identity with a number of immunoglobin superfamily proteins. The identity information used for ClustalW analysis is presented in Table

TABLE 7K
BLAST results for NOV7

| Gene Index/ | Protein/ | Length | Identity | Positives | Ex- |
|---|---|---|---|---|---|
| Identifier | Organism | (aa) | (%) | (%) | pect |
| gi\|10434261\|dbj\| | unnamed | 571 | 514/572 | 518/572 | 0.0 |
| BAB1 4192.1\| | protein | | (89%) | (89%) | |
| (AK022708) | product | | | | |
| | Homo | | | | |
| | sapiens | | | | |
| gi\|8922705\|ref\| | hypo- | 410 | 367/409 | 367/409 | 0.0 |
| NP 06 0710.1\| | thetical | | (89%) | (89%) | |
| | protein | | | | |
| | FLJ10845 | | | | |
| | Homo | | | | |
| | sapiens | | | | |
| gi\|14017951\|dbj\| | KIAA1867 | 779 | 283/622 | 372/622 | e−146 |
| BAB4 7496.1\| | protein | | (45%) | (59%) | |
| (AB058770) | Homo | | | | |
| | sapiens | | | | |
| gi\|13639054\|ref\| | hypo- | 296 | 254/296 | 254/296 | e−142 |
| XP 0 02177.3\| | thetical | | (85%) | (85%) | |
| | protein | | | | |
| | FLJ10845 | | | | |
| | Homo | | | | |
| | sapiens | | | | |
| gi\|12043535\|emb\| | dumb- | 959 | 144/462 | 212/462 | 2e−46 |
| CAB9 6574.2\| | founded | | (31% | (45%) | |
| (AJ289882) | Droso- | | | | |
| | phila- | | | | |
| | melano- | | | | |
| | gaster | | | | |

This information is presented graphically in the multiple sequence alignment given in Table 7L (with NOV7a being shown on line 1) as a ClustalW analysis comparing NOV7 with related protein sequences.

[image]

[image]

[image]

DOMAIN results for NOV7a were collected from the Conserved Domain Database (CDD) with Reverse Position Specific BLAST. This BLAST samples domains found in the Smart and Pfam collections. The results for NOV7a are listed in Table 7M with the statistics and domain description.

TABLE 7M
DOMAIN results for NOV7

| PSSMs producing | Score | E |
|---|---|---|
| significant alignments: | (bits) | value |
| gnl\|Smart\|smart00409 | 43.9 | 3e−05 |
| IG, Immunoglobulin | | |
| gnl\|Smart\|smart00409 | 43.5 | 4e−05 |
| IG, Immunoglobulin | | |
| gnl\|Smart\|smart00409 | 40.8 | 2e−04 |
| IG, Immunoglobulin | | |
| gnl\|Pfam\|pfam00047 | 37.4 | 0.003 |
| ig, Immunoglobulin domain | | |
| gnl\|Smart\|smart00408 | 37.0 | 0.004 |
| IGc2, Immunoglobulin C-2 Type | | |
| gnl\|Smart\|smart00408 | 35.8 | 0.008 |
| IGc2, Immunoglobulin C-2 Type | | |

The alignment with smart00409 is shown in Table 7N. The similarity of NOV7 with the immmunoglobulin domain indicates that the NOV7 sequence has properties similar to those of other proteins known to contain this domain.

[image]

Based on sequence homology with other immunoglobulin superfamily members, as well as domain information, the disclosed NOV7 proteins are likely to be involved in protein-protein and protein-ligand interactions.

The nucleic acids and proteins of NOV7 are useful in potential therapeutic applications implicated in various pathological disorders, described further below. For example, a cDNA encoding the immunoglobulin superfamily-like protein may be useful in gene therapy, and the immunoglobulin superfamily-like protein may be useful when administered to a subject in need thereof.

The nucleic acids and proteins of the invention have applications in the diagnosis and/or treatment of various diseases and disorders. For example, the compositions of the present invention will have efficacy for the treatment of patients suffering from, CNS diseases such as Parkinson's disease and Alzheimer's disease, corpus callosum agenesis, retardation, adducted thumbs, spastic paraparesis, hydrocephalus, agenesis or hypoplasia of corpus callosum, primitive neuroectodermal tumors, human neuroteratocarcinoma and other cancers, human type 2 lissencephaly, recurrent seizures and hippocampal sclerosis as well as other diseases, disorders and conditions.

The polypeptides can be used as immunogens to produce antibodies specific for the invention, and as vaccines. They can also be used to screen for potential agonist and antagonist compounds. For example, a cDNA encoding the immunoglobulin superfamily-protein may be useful in gene therapy, and the receptor-like protein may be useful when administered to a subject in need thereof. The novel nucleic acid encoding immunoglobulin superfamily-like proteins, and the immunoglobulin superfamily-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV7 protein has multiple hydrophilic regions, each of which can be used as an immunogen. This novel protein also has value in development of powerful assay system for functional analysis of various human disorders, which will help in understanding of pathology of the disease and development of new drug targets for various disorders.

TaqMan data are presented in Example 1.

### EXAMPLE 1

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR; TAQMAN®). RTQ PCR was performed on a Perkin-Elmer Biosystems ABI PRISM® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources) and Panel 4 (containing cells and cell lines from normal cells and cells related to inflammatory conditions).

First, the RNA samples were normalized to constitutively expressed genes such as β-actin and GAPDH. RNA (˜50 ng total or ˜1 ng polyA+) was converted to cDNA using the TAQMAN® Reverse Transcription Reagents Kit (PE Biosystems, Foster City, Calif.; Catalog No. N808-0234) and random hexamers according to the manufacturer's protocol. Reactions were performed in 20 ul and incubated for 30 min. at 48° C. cDNA (5 ul) was then transferred to a separate plate for the TAQMAN® reaction using β-actin and GAPDH TAQMAN® Assay Reagents (PE Biosystems; Catalog Nos. 4310881E and 4310884E, respectively) and TAQMAN® universal PCR Master Mix (PE Biosystems; Catalog No. 4304447) according to the manufacturer's protocol. Reactions were performed in 25 ul using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. The average CT values obtained for β-actin and GAPDH were used to normalize RNA samples. The RNA sample generating the highest CT value required no further diluting, while all other samples were diluted relative to this sample according to their β-actin /GAPDH average CT values.

Normalized RNA (5 ul) was converted to cDNA and analyzed via TAQMAN® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration =250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1X TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

In the results for Panel 1, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue). In addition, RNA and cDNA samples were obtained from various human tissues derived from autopsies performed on elderly people or sudden death victims (accidents, etc.). These tissue were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s: 1 8s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-I 8 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10-5 M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10-5 M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD1106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C overnight. The precipitated RNA was spun down at 9,000 rpm for 15 min in a Sorvall SS34 rotor and washed in 70% ethanol. The pellet was redissolved in 300 pl of RNAse-free water and 35 μl buffer (Promega) 5 μl DTT, 7 μl RNAsin and 8 VlI DNAse were added. The tube was incubated at 37 degrees C for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

NOV1a

Expression of gene 20421338.0.44 (NOV1a) was assessed using the primer-probe set Ag689, described in Table A. Results of the RTQ-PCR runs are shown in Tables B, C, D, and E.

| | | | | |
|---|---|---|---|---|
| | | | | Start |
| Primers | Sequences | TM | Length | Position |

(SEQ ID NO:98)

| | | | | |
|---|---|---|---|---|
| Forward | 5′-GATGTGCACCACCCTCCT | 59.5 | 19 | 240 |

A-3′
(SEQ ID NO:99)

| | | | | |
|---|---|---|---|---|
| Probe | TET-5′-AGTGACCCTGTCCA | 69.7 | 25 | 260 |

TTGAGCCACAG-3′-TAMRA
(SEQ ID NO:100)

| | | | | |
|---|---|---|---|---|
| Reverse | 5′-CCTGGCAGGTAAAGACAA | 58.8 | 20 | 305 |

TTGAGCCACAG-3′-TAMRA

TABLE B
Panel 1.2

| | Relative Expression (%) | |
|---|---|---|
| Tissue Name | 1.2tm884t_ag689 | 1.2tm897t_ag689 |
| Endothelial cells | 13.0 | 12.8 |
| Endothelial cells (treated) | 8.0 | 7.3 |
| Pancreas | 5.4 | 5.7 |
| Pancreatic ca. CAPAN 2 | 21.8 | 7.6 |
| Adrenal Gland (new lot*) | 9.9 | 19.6 |
| Thyroid | 9.2 | 8.8 |
| Salivary gland | 5.9 | 6.2 |
| Pituitary gland | 4.7 | 5.1 |
| Brain (fetal) | 6.3 | 9.2 |
| Brain (whole) | 2.5 | 2.8 |
| Brain (amygdala) | 0.8 | 1.3 |
| Brain (cerebellum) | 1.1 | 0.9 |
| Brain (hippocampus) | 6.6 | 2.2 |
| Brain (thalamus) | 1.4 | 2.5 |
| Cerebral Cortex | 1.7 | 3.3 |
| Spinal cord | 2.3 | 2.8 |
| CNS ca. (glio/astro) U87-MG | 34.2 | 28.3 |
| CNS ca. (glio/astro) U-118-MG | 25.7 | 22.7 |
| CNS ca. (astro) SW1783 | 12.2 | 11.8 |
| CNS ca.* (neuro; met) SK-N-AS | 39.5 | 39.0 |
| CNS ca. (astro) SF-539 | 26.8 | 29.5 |
| CNS ca (astro) SNB-75 | 27.5 | 25.5 |
| CNS ca. (glio) SNB-19 | 57.8 | 41.5 |
| CNS ca. (glio) U251 | 97.3 | 43.2 |
| CNS ca. (glio) SF-295 | 52.8 | 38.4 |
| Heart | 11.8 | 12.1 |
| Skeletal Muscle (new lot*) | 6.7 | 7.3 |
| Bone marrow | 0.4 | 0.5 |
| Thymus | 2.3 | 1.8 |
| Spleen | 2.5 | 2.5 |
| Lymph node | 3.5 | 4.6 |
| Colorectal | 1.1 | 1.0 |
| Stomach | 4.9 | 6.0 |
| Small intestine | 8.0 | 8.1 |
| Colon ca. SW480 | 5.5 | 2.6 |
| Colon ca.* (SW480 met)SW620 | 6.3 | 4.2 |
| Colon ca. HT29 | 2.2 | 2.0 |
| Colon ca. HCT-116 | 4.8 | 3.6 |
| Colon ca. CaCo-2 | 17.9 | 14.8 |
| 83219 CC Well to | 1.4 | 2.0 |
| Mod Diff (ODO3866) | | |
| Colon ca. HCC-2998 | 1.8 | 1.7 |
| Gastric ca* (liver met) NCI-N87 | 2.5 | 2.4 |
| Bladder | 12.9 | 14.8 |
| Trachea | 2.2 | 3.6 |
| Kidney | 12.9 | 11.9 |
| Kidney (fetal) | 44.8 | 44.8 |
| Renal ca. 786-0 | 64.6 | 36.1 |
| Renal ca. A498 | 54.7 | 57.0 |
| Renal ca. RXF 393 | 12.1 | 14.4 |
| Renal ca. ACHN | 66.9 | 60.3 |
| Renal ca. UO-31 | 95.3 | 51.4 |
| Renal ca. TK-10 | 77.4 | 50.0 |
| Liver | 4.2 | 4.4 |
| Liver (fetal) | 2.0 | 2.0 |
| Liver ca. (hepatoblast) HepG2 | 0.0 | 0.0 |
| Lung | 5.4 | 6.3 |
| Lung (fetal) | 8.0 | 8.4 |
| Lung ca. (small cell) LX-1 | 3.5 | 1.9 |
| Lung ca. (small cell) NCI-H69 | 6.6 | 5.2 |
| Lung ca. (s. cell var.) SHP-77 | 0.0 | 0.2 |
| Lung ca. (large cell) NCI-H460 | 29.5 | 28.3 |
| Lung ca. (non-sm. cell) A549 | 8.8 | 7.5 |
| Lung ca. (non-s.cell) NCI-H23 | 10.1 | 5.1 |
| Lung ca (non-s.cell) HOP-62 | 50.0 | 36.3 |
| Lung ca. (non-s.cl) NCI-H522 | 6.6 | 6.1 |
| Lung ca. (squam.) SW 900 | 10.1 | 8.1 |
| Lung ca. (squam.) NCI-H596 | 10.8 | 17.1 |
| Mammary gland | 22.7 | 25.7 |
| Breast ca.* (pl. effusion) MCF-7 | 0.0 | 0.0 |
| Breast ca.* (pl. ef) MDA-MB-231 | 11.2 | 12.4 |
| Breast ca.* (pl. effusion) T47D | 2.6 | 2.6 |
| Breast ca. BT-549 | 24.0 | 26.8 |
| Breast ca. MDA-N | 26.8 | 23.5 |
| Ovary | 22.4 | 19.9 |
| Ovarian ca. OVCAR-3 | 56.3 | 63.3 |
| Ovarian ca. OVCAR-4 | 57.4 | 40.9 |
| Ovarian ca. OVCAR-5 | 50.0 | 52.5 |
| Ovarian ca. OVCAR-8 | 19.9 | 15.4 |
| Ovarian ca. IGROV-1 | 100.0 | 100.0 |
| Ovarian ca.* (ascites) SK-OV-3 | 97.9 | 88.3 |
| Uterus | 10.4 | 8.5 |
| Placenta | 13.5 | 14.4 |
| Prostate | 3.0 | 3.3 |
| Prostate ca.* (bone met)PC-3 | 41.5 | 27.5 |
| Testis | 4.2 | 4.1 |
| Melanoma Hs688(A).T | 16.7 | 11.4 |
| Melanoma* (met) Hs688(B).T | 14.9 | 14.1 |
| Melanoma UACC-62 | 52.8 | 54.0 |
| Melanoma M14 | 24.3 | 13.4 |
| Melanoma LOX IMVI | 21.9 | 11.4 |
| Melanoma* (met) SK-MEL-S | 10.6 | 6.6 |
| Adipose | 2.2 | 2.6 |

TABLE C
Panel 2D

| | Relative Expression (%) | |
|---|---|---|
| Tissue Name | 2Dtm2692t_ag689 | 2Dtm2919t_ag689 |
| Normal Colon GENPAK 061003 | 25.9 | 27.0 |
| 83219 CC Well to Mod Diff (ODO3866) | 9.9 | 7.4 |
| 83220 CC NAT (ODO3866) | 6.9 | 2.7 |
| 83221 CC Gr.2 rectosigmoid (ODO3868) | 4.0 | 2.4 |
| 83222 CC NAT (ODO3868) | 4.5 | 3.7 |
| 83235 CC Mod Diff (ODO3920) | 3.4 | 3.0 |
| 83236 CC NAT (ODO3920) | 5.8 | 6.2 |
| 83237 CC Gr.2 ascend colon (ODO3921) | 22.5 | 16.7 |
| 83238 CC NAT (ODO3921) | 8.2 | 5.9 |
| 83241 CC from Partial Hepatectomy (ODO4309) | 18.9 | 21.6 |
| 83242 Liver NAT (ODO4309) | 3.6 | 2.2 |
| 87472 Colon mets to lung (OD04451-01) | 8.1 | 5.8 |
| 87473 Lung NAT (OD04451-02) | 7.3 | 4.7 |
| Normal Prostate Clontech A+ 6546-1 | 6.0 | 3.8 |
| 84140 Prostate Cancer (OD04410) | 12.2 | 11.7 |
| 84141 Prostate NAT (OD04410) | 13.8 | 17.8 |
| 87073 Prostate Cancer (OD04720-01) | 11.3 | 9.0 |
| 87074 Prostate NAT (OD04720-02) | 19.2 | 18.2 |
| Normal Lung GENPAK 061010 | 18.9 | 17.7 |
| 83239 Lung Met to Muscle (ODO4286) | 45.7 | 35.1 |
| 83240 Muscle NAT (ODO4286) | 13.0 | 10.8 |
| 84136 Lung Malignant Cancer (OD03126) | 18.9 | 12.4 |
| 84137 Lung NAT (OD03126) | 26.6 | 17.7 |
| 84871 Lung Cancer (OD04404) | 46.7 | 36.1 |
| 84872 Lung NAT (OD04404) | 27.0 | 20.9 |
| 84875 Lung Cancer (OD04565) | 21.8 | 18.3 |
| 84876 Lung NAT (OD04565) | 14.7 | 13.1 |
| 85950 Lung Cancer (OD04237-01) | 6.4 | 4.4 |
| 85970 Lung NAT (OD04237-02) | 23.3 | 22.8 |
| 83255 Ocular Mel Met to Liver (ODO4310) | 14.1 | 14.0 |
| 83256 Liver NAT (ODO4310) | 3.4 | 2.4 |
| 84139 Melanoma Mets to Lung (OD04321) | 39.8 | 45.1 |
| 84138 Lung NAT (OD04321) | 17.4 | 11.7 |
| Normal Kidney GENPAK 061008 | 44.4 | 42.9 |
| 83786 Kidney Ca, Nuclear grade 2 (OD04338) | 34.6 | 29.3 |
| 83787 Kidney NAT (OD04338) | 26.1 | 25.5 |
| 83788 Kidney Ca Nuclear grade ½ (OD04339) | 28.9 | 23.7 |
| 83789 Kidney NAT (OD04339) | 27.9 | 29.5 |
| 83790 Kidney Ca, Clear cell type (OD04340) | 44.8 | 34.9 |
| 83791 Kidney NAT (OD04340) | 37.1 | 37.6 |
| 83792 Kidney Ca, Nuclear grade 3 (OD04348) | 39.2 | 38.7 |
| 83793 Kidney NAT (OD04348) | 29.1 | 35.1 |
| 87474 Kidney Cancer (OD04622-01) | 37.6 | 37.1 |
| 87475 Kidney NAT (OD04622-03) | 6.8 | 4.9 |
| 85973 Kidney Cancer (OD04450-01) | 28.3 | 29.9 |
| 85974 Kidney NAT (OD04450-03) | 26.6 | 25.9 |
| Kidney Cancer Clontech 8120607 | 27.4 | 20.3 |
| Kidney NAT Clontech 8120608 | 13.7 | 13.6 |
| Kidney Cancer Clontech 8120613 | 2.4 | 1.4 |
| Kidney NAT Clontech 8120614 | 17.0 | 12.9 |
| Kidney Cancer Clontech 9010320 | 50.3 | 49.3 |
| Kidney NAT Clontech 9010321 | 31.4 | 29.1 |
| Normal Uterus GENPAK 061018 | 11.9 | 9.9 |
| Uterus Cancer GENPAK 064011 | 18.3 | 11.3 |
| Normal Thyroid Clontech A+ 6570-1 | 7.5 | 7.5 |
| Thyroid Cancer GENPAK 064010 | 22.2 | 15.6 |
| Thyroid Cancer INVITROGEN A302152 | 6.0 | 4.5 |
| Thyroid NAT INVITROGEN A302153 | 12.3 | 7.6 |
| Normal Breast GENPAK 061019 | 33.0 | 24.8 |
| 84877 Breast Cancer (OD04566) | 2.9 | 5.1 |
| 85975 Breast Cancer (OD04590-01) | 15.9 | 14.9 |
| 85976 Breast Cancer Mets (OD04590-03) | 26.4 | 20.0 |
| 87070 Breast Cancer Metastasis (OD04655-05) | 2.7 | 1.9 |
| GENPAK Breast Cancer 064006 | 17.6 | 14.3 |
| Breast Cancer Res. Gen. 1024 | 25.5 | 27.0 |
| Breast Cancer Clontech 9100266 | 16.3 | 19.6 |
| Breast NAT Clontech 9100265 | 24.0 | 25.0 |
| Breast Cancer INVITROGEN A209073 | 33.9 | 32.8 |
| Breast NAT INVITROGEN A2090734 | 25.3 | 30.8 |
| Normal Liver GENPAK 061009 | 1.9 | 1.5 |
| Liver Cancer GENPAK 064003 | 1.2 | 1.0 |
| Liver Cancer Research Genetics RNA 1025 | 3.1 | 3.6 |
| Liver Cancer Research Genetics RNA 1026 | 9.8 | 8.8 |
| Paired Liver Cancer Tissue Research | 6.2 | 3.6 |
| Genetics RNA 6004-T | | |
| Paired Liver Tissue Research Genetics | 2.8 | 2.0 |
| RNA 6004-N | | |
| Paired Liver Cancer Tissue Research | 9.5 | 10.7 |
| Genetics RNA 6005-T | | |
| Paired Liver Tissue Research Genetics | 3.4 | 4.3 |
| RNA 6005-N | | |
| Normal Bladder GENPAK 061001 | 23.3 | 17.9 |
| Bladder Cancer Research Genetics RNA 1023 | 11.3 | 9.2 |
| Bladder Cancer INVITROGEN A302173 | 7.1 | 12.6 |
| 87071 Bladder Cancer (OD04718-01) | 9.5 | 5.7 |
| 87072 Bladder Normal Adjacent (OD04718-03) | 39.0 | 28.7 |
| Normal Ovary Res. Gen. | 42.3 | 41.2 |
| Ovarian Cancer GENPAK 064008 | 100.0 | 100.0 |
| 87492 Ovary Cancer (OD04768-07) | 71.7 | 62.0 |
| 87493 Ovary NAT (OD04768-08) | 21.8 | 19.2 |
| Normal Stomach GENPAK 061017 | 11.6 | 9.4 |
| Gastric Cancer Clontech 9060358 | 5.9 | 5.1 |
| NAT Stomach Clontech 9060359 | 5.6 | 3.8 |
| Gastric Cancer Clontech 9060395 | 23.0 | 18.2 |
| NAT Stomach Clontech 9060394 | 12.2 | 9.3 |
| Gastric Cancer Clontech 9060397 | 22.7 | 16.3 |
| NAT Stomach Clontech 9060396 | 3.1 | 1.8 |
| Gastric Cancer GENPAK 064005 | 10.7 | 7.4 |

TABLE D
Panel 3D

| | Relative |
|---|---|
| | Expression (%) |
| Tissue Name | 3dx4tm6101t_ag689₁₃ b2 |
| 94905_Daoy_Medulloblastoma/Cerebellum_sscDNA | 10.9 |
| 94906_TE671_Medulloblastom/Cerebellum_sscDNA | 5.7 |
| 94907_D283 Med_Medulloblastoma/Cerebellum_sscDNA | 39.7 |
| 94908_PFSK-1_Primitive Neuroectodermal/Cerebellum_sscDNA | 1.3 |
| 94909_XF-498_CNS_sscDNA | 39.1 |
| 94910_SNB-78_CNS/glioma_sscDNA | 12.8 |
| 94911_SF-268_CNS/glioblastoma_sscDNA | 7.0 |
| 94912_T98G_Glioblastoma_sscDNA | 26.5 |
| 96776_SK-N-SH_Neuroblastoma (metastasis)_sscDNA | 7.3 |
| 94913_SF-295_CNS/glioblastoma_sscDNA | 34.1 |
| 94914_Cerebellum_sscDNA | 0.4 |
| 96777_Cerebellum_sscDNA | 0.1 |
| 94916_NCI-H292_Mucoepidermoid lung carcinoma_sscDNA | 13.0 |
| 94917_DMS-114_Small cell lung cancer_sscDNA | 3.3 |
| 94918_DMS-79_Small cell lung cancer/neuroendocrine_sscDNA | 0.5 |
| 94919_NCI-H146_Small cell lung cancer/neuroendocrine_sscDNA | 0.0 |
| 94920_NCI-H526_Small cell lung cancer/neuroendocrine_sscDNA | 2.6 |
| 94921_NCI-N417_Small cell lung cancer/neuroendocrine_sscDNA | 3.7 |
| 94923_NCI-H82_Small cell lung cancer/neuroendocrine_sscDNA | 4.7 |
| 94924_NCI-H157_Squamous cell lung cancer (metastasis)_sscDNA | 19.0 |
| 94925_NCI-H1155_Large cell lung cancer/neuroendocrine_sscDNA | 1.3 |
| 94926_NCI-H1299_Large cell lung cancer/neuroendocrine_sscDNA | 20.4 |
| 94927_NCI-H727_Lung carcinoid_sscDNA | 2.0 |
| 94928_NCI-UMC-11_Lung carcinoid_sscDNA | 0.0 |
| 94929_LX-1_Small cell lung cancer_sscDNA | 1.2 |
| 94930_Colo-205_Colon cancer_sscDNA | 0.0 |
| 94931_KM12_Colon cancer_sscDNA | 0.0 |
| 94932_KM20L2_Colon cancer_sscDNA | 0.7 |
| 94933_NCI-H716_Colon cancer_sscDNA | 0.0 |
| 94935_SW-48_Colon adenocarcinoma_sscDNA | 0.3 |
| 94936_SW1116_Colon adenocarcinoma_sscDNA | 0.0 |
| 94937_LS 174T_Colon adenocarcinoma_sscDNA | 0.0 |
| 94938_SW-948_Colon adenocarcinoma_sscDNA | 1.1 |
| 94939_SW-480_Colon adenocarcinoma_sscDNA | 0.7 |
| 94940_NCI-SNU-5_Gastric carcinoma_sscDNA | 2.7 |
| 94941_KATO III_Gastric carcinoma_sscDNA | 100.0 |
| 94943_NCI-SNU-16_Gastric carcinoma_sscDNA | 10.1 |
| 94944_NCI-SNU-1_Gastric carcinoma_sscDNA | 0.0 |
| 94946_RF-1_Gastric adenocarcinoma_sscDNA | 0.0 |
| 94947_RF-48_Gastric adenocarcinoma_sscDNA | 0.0 |
| 96778_MKN-45_Gastric carcinoma_sscDNA | 0.9 |
| 94949_NCI-N87_Gastric carcinoma_sscDNA | 0.4 |
| 94951_OVCAR-5_Ovarian carcinoma_sscDNA | 1.2 |
| 94952_RL95-2_Uterine carcinoma_sscDNA | 3.3 |
| 94953_HelaS3_Cervical adenocarcinoma_sscDNA | 2.4 |
| 94954_Ca Ski_Cervical epidermoid carcinoma (metastasis)_sscDNA | 12.5 |
| 94955_ES-2_Ovarian clear cell carcinoma_sscDNA | 4.1 |
| 94957_Ramos/6h stim_“; Stimulated with PMA/ionomycin 6h_sscDNA | 0.0 |
| 94958_Ramos/14h stim_“; Stimulated with PMA/ionomycin 14h_sscDNA | 0.0 |
| 94962_MEG-01_Chronic myelogenous leukemia (megokaryoblast)_sscDNA | 1.3 |
| 94963_Raji_Burkitt's lymphoma_sscDNA | 0.0 |
| 94964_Daudi_Burkitt's lymphoma_sscDNA | 0.0 |
| 94965_U266_B-cell plasmacytoma/myeloma_sscDNA | 0.0 |
| 94968_CA46_Burkitt's lymphoma_sscDNA | 0.0 |
| 94970_RL_non-Hodgkin's B-cell lymphoma_sscDNA | 0.0 |
| 94972_JM1_pre-B-cell lymphoma/leukemia_sscDNA | 0.0 |
| 94973_Jurkat_T cell leukemia_sscDNA | 1.8 |
| 94974_TF-1 Erythroleukemia_sscDNA | 0.0 |
| 94975_HUT 78_T-cell lymphoma_sscDNA | 0.0 |
| 94977_U937_Histiocytic lymphoma_sscDNA | 0.0 |
| 94980_KU-812_Myelogenous leukemia_sscDNA | 0.0 |
| 94981_769-P Clear cell renal carcinoma_sscDNA | 30.2 |
| 94983_Caki-2_Clear cell renal carcinoma_sscDNA | 14.1 |
| 94984_SW 839_Clear cell renal carcinoma_sscDNA | 4.3 |
| 94986_G401_Wilms' tumor_sscDNA | 9.1 |
| 94987_Hs766T_Pancreatic carcinoma (LN metastasis)_sscDNA | 19.6 |
| 94988_CAPAN-1_Pancreatic adenocarcinoma (liver metastasis)_sscDNA | 4.2 |
| 94989_SU86.86_Pancreatic carcinoma (liver metastasis)_sscDNA | 7.1 |
| 94990_BxPC-3_Pancreatic adenocarcinoma_sscDNA | 2.0 |
| 94991_HPAC_Pancreatic adenocarcinoma_sscDNA | 1.8 |
| 94992_MIA PaCa-2_Pancreatic carcinoma_sscDNA | 4.0 |
| 94993_CFPAC-1_Pancreatic ductal adenocarcinoma_sscDNA | 26.0 |
| 94994_PANC-1_Pancreatic epithelioid ductal carcinoma_sscDNA | 12.1 |
| 94996_T24_Bladder carcinma (transitional cell)_sscDNA | 0.0 |
| 94997_5637_Bladder carcinoma_sscDNA | 3.5 |
| 94998_HT-1197_Bladder carcinoma_sscDNA | 0.6 |
| 94999_UM-UC-3_Bladder carcinma (transitional cell)_sscDNA | 1.7 |
| 95000_A204_Rhabdomyosarcoma_sscDNA | 5.8 |
| 95001_HT-1080_Fibrosarcoma_sscDNA | 15.4 |
| 95002_MG-63_Osteosarcoma (bone)_sscDNA | 14.5 |
| 95003_SK-LMS-1_Leiomyosarcoma (vulva)_sscDNA | 9.5 |
| 95004_SJRH30_Rhabdomyosarcoma (met to bone marrow)_sscDNA | 4.8 |
| 95005_A431_Epidermoid carcinoma_sscDNA | 2.9 |
| 95007_WM266-4_Melanoma_sscDNA | 22.6 |
| 95010_DU 145_Prostate carcinoma (brain metastasis)_sscDNA | 0.0 |
| 95012_MDA-MB-468_Breast adenocarcinoma_sscDNA | 7.7 |
| 95013_SCC-4_Squamous cell carcinoma of tongue_sscDNA | 0.0 |
| 95014_SCC-9_Squamous cell carcinoma of tongue_sscDNA | 0.0 |
| 95015_SCC-15_Squamous cell carcinoma of tongue_sscDNA | 0.0 |
| 95017_CAL 27_Squamous cell carcinoma of tongue_sscDNA | 5.6 |

TABLE E
Panel 4D

| | Relative |
|---|---|
| | Expression |
| | (%) |
| Tissue Name | 4dx4tm4997t_ag689_a2 |
| 93768_Secondary Th1_anti-CD28/anti-CD3 | 0.0 |
| 93769_Secondary Th2_anti-CD28/anti-CD3 | 0.0 |
| 93770_Secondary Tr1_anti-CD28/anti-CD3 | 0.0 |
| 93573_Secondary Th1_resting day 4-6 in IL-2 | 0.0 |
| 93572_Secondary Th2_resting day 4-6 in IL-2 | 0.0 |
| 93571_Secondary Tr1_resting day 4-6 in IL-2 | 0.0 |
| 93568_primary Th1_anti-CD28/anti-CD3 | 0.1 |
| 93569_primary Th2_anti-CD28/anti-CD3 | 0.0 |
| 93570_pnimary Tr1_anti-CD28/anti-CD3 | 0.2 |
| 93565_primary Th1_resting dy 4-6 in IL-2 | 0.0 |
| 93566_primary Th2_resting dy 4-6 in IL-2 | 0.0 |
| 93567_primary Tr1_resting dy 4-6 in IL-2 | 0.0 |
| 93351_CD45RA CD4 lymphocyte_anti-CD28/anti-CD3 | 19.7 |
| 93352_CD45RO CD4 lymphocyte_anti-CD28/anti-CD3 | 0.0 |
| 93251_CD8 Lymphocytes_anti-CD28/anti-CD3 | 0.0 |
| 93353_chronic CD8 Lymphocytes 2ry_resting dy 4-6 in IL-2 | 0.0 |
| 93574_chronic CD8 Lymphocytes 2ry_activated CD3/CD28 | 0.0 |
| 93354_CD4_none | 0.0 |
| 93252_Secondary Th1/Th2/Tr1_anti-CD95 CH11 | 0.0 |
| 93103_LAK cells_resting | 0.0 |
| 93788_LAK cells_IL-2 | 0.0 |
| 93787_LAK cells_IL-2 + IL-12 | 0.0 |
| 93789_LAK cells_IL-2 + IFN gamma | 0.0 |
| 93790_LAK cells_IL-2 + IL-18 | 0.0 |
| 93104_LAK cells_PMA/ionomycin and IL-18 | 0.0 |
| 93578_NK Cells IL-2_resting | 0.0 |
| 93109_Mixed Lymphocyte Reaction_Two Way MLR | 0.0 |
| 93110_Mixed Lymphocyte Reaction_Two Way MLR | 0.0 |
| 93111_Mixed Lymphocyte Reaction_Two Way MLR | 0.0 |
| 93112_Mononuclear Cells (PBMCs)_resting | 0.0 |
| 93113_Mononuclear Cells (PBMCs)_PWM | 0.0 |
| 93114_Mononuclear Cells (PBMCs)_PHA-L | 0.1 |
| 93249_Ramos (B cell)_none | 0.0 |
| 93250_Ramos (B cell)_ionomycin | 0.0 |
| 93349_B lymphocytes_PWM | 0.0 |
| 93350_B lymphoytes_CD40L and IL-4 | 0.2 |
| 92665_EOL-1 (Eosinophil)_dbcAMP differentiated | 0.0 |
| 93248_EOL-1 (Eosinophil)_dbcAMP/PMAionomycin | 0.0 |
| 93356_Dendritic Cells_none | 0.0 |
| 93355_Dendritic Cells_LPS 100 ng/ml | 0.0 |
| 93775_Dendritic Cells_anti-CD40 | 0.0 |
| 93774_Monocytes_resting | 0.0 |
| 93776_Monocytes_LPS 50 ng/ml | 0.0 |
| 93581_Macrophages_resting | 0.0 |
| 93582_Macrophages_LPS 100 ng/ml | 0.0 |
| 93098_HUVEC (Endothelial)_none | 36.5 |
| 93099_HUVEC (Endothelial)_starved | 35.4 |
| 93100_HUVEC (Endothelial)_IL-1b | 5.9 |
| 93779_HUVEC (Endothelial)_IFN gamma | 9.9 |
| 93102_HUVEC (Endothelial)_TNF alpha + IFN gamma | 17.1 |
| 93101_HUVEC (Endothelial)_TNF alpha + IL4 | 11.2 |
| 93781_HUVEC (Endothelial)_IL-11 | 7.2 |
| 93583_Lung Microvascular Endothelial Cells_none | 20.0 |
| 93584_Lung Microvascular Endothelial Cells_TNFa (4 ng/ml) and IL1b (1 | 19.4 |
| ng/ml) | |
| 92662_Microvascular Dermal endothelium_none | 34.5 |
| 92663_Microsvasular Dermal endothelium_TNFa (4 ng/ml) and IL1b (1 ng/ml) | 21.9 |
| 93773_Bronchial epithelium_TNFa (4 ng/ml) and IL1b (1 ng/ml)** | 37.1 |
| 93347_Small Airway Epithelium_none | 16.4 |
| 93348_Small Airway Epithelium_TNFa (4 ng/ml) and IL1b (1 ng/ml) | 68.0 |
| 92668_Coronery Artery SMC_resting | 47.5 |
| 92669_Coronery Artery SMC_TNFa (4 ng/ml) and IL1b (1 ng/ml) | 25.5 |
| 93107_astrocytes_resting | 39.3 |
| 93108_astrocytes_TNFa (4 ng/ml) and IL1b (1 ng/ml) | 21.8 |
| 92666_KU-812 (Basophil)_resting | 0.0 |
| 92667_KU-812 (Basophil)_PMA/ionoycin | 0.0 |
| 93579_CCD1106 (Keratinocytes)_none | 18.7 |
| 93580_CCD1106 (Keratinocytes)_TNFa and IFNg** | 4.4 |
| 93791_Liver Cirrhosis | 2.4 |
| 93792_Lupus Kidney | 2.8 |
| 93577_NCI-H292 | 14.9 |
| 93358_NCI-H292_IL-4 | 16.6 |
| 93360_NCI-H292_IL-9 | 19.2 |
| 93359_NCI-H292_IL-13 | 9.3 |
| 93357_NCI-H292_IFN gamma | 9.9 |
| 93777_HPAEC_- | 9.2 |
| 93778_HPAEC_IL-1 beta/TNA alpha | 13.4 |
| 93254_Normal Human Lung Fibroblast_none | 39.7 |
| 93253_Normal Human Lung Fibroblast_TNFa (4 ng/ml) and IL-lb (1 ng/ml) | 29.9 |
| 93257_Normal Human Lung Fibroblast_IL-4 | 70.9 |
| 93256_Normal Human Lung Fibroblast_IL-9 | 58.7 |
| 93255_Normal Human Lung Fibroblast_IL-13 | 34.4 |
| 93258_Normal Human Lung Fibroblast_IFN gamma | 77.0 |
| 93106_Dermal Fibroblasts CCD1070_resting | 100.0 |
| 93361_Dermal Fibroblasts CCD1070_TNF alpha 4 ng/ml | 80.5 |
| 93105_Dermal Fibroblasts CCD1070_IL-1 beta 1 ng/ml | 40.6 |
| 93772_dermal fibroblast_IFN gamma | 22.7 |
| 93771_dermal fibroblast_IL-4 | 48.7 |
| 93259_IBD Colitis 1** | 2.8 |
| 93260_IBD Colitis 2 | 0.4 |
| 93261_IBD Crohns | 0.8 |
| 735010_Colon_normal | 2.5 |
| 735019_Lung_none | 27.5 |
| 64028-1_Thymus_none | 17.2 |
| 64030-1_Kidney_none | 8.3 |

Panel 1.2 Summary: Expression of NOV1a in this panel is highest in a number of cancer cell lines, including ovarian cancer, prostate cancer, melanoma, renal carcinoma, and CNS cancers (CT values<29). Moderate to low expression of this gene is detected in most normal tissues with the highest expression in fetal kidney (CT values 29-35). The results obtained from the two separate RTQ-PCR experiments using Ag689 are roughly in agreement. Thus, the data reveal that this gene is expressed quite highly in samples derived from cancer cell lines and not expressed as highly in samples derived from normal tissues. In addition, within the normal kidney samples there seems to be a consistent difference between adult and fetal tissues. Taking into account that cell lines are, on the whole, more proliferative than tissues and that fetal tissues also show a higher proliferative compartment than their adult counterparts, it is speculated that this gene might be involved in cell proliferation. Antibodies or small molecule drugs targeting NOV1a might therefore be useful for the treatment of diseases that show increased cellular proliferation, such as cancer.

Panel 2D Summary: The level of expression of NOV1a appeared lower in this panel than was observed for Panel 1.2 (CT values 30-35). The results obtained from the two separate RTQ-PCR experiments using Ag689 are roughly in agreement. The data in panel 2D shows a wide expression profile across many of the samples. Specifically, however, there appears to be preponderance for NOV1a expression in certain cancer samples when compared to their normal adjacent controls; specific examples include gastric cancer, ovarian cancer and a couple of colon cancers. Therefore, inhibition of the NOVI a gene product might provide an effective treatment for gastric cancer, ovarian cancer and certain colon cancers. In addition, this gene may be useful as a marker for diagnosing these diseases.

Panel 3D Summary: Expression of NOV1a in panel 3D was highest in a gastric cancer cell line. Consistent to what was observed with panel 1.2D, expression of this gene was detected in a number of cancer cell lines. Analysis of the samples in panel 3D reveals that this gene is expressed by a particular set of cancer cell lines, but not all. Particularly, the NOVI a transcript seems to be curiously absent from leukemia/lymphoma cell lines and colon cancer cell lines, given its broad expression pattern. The lack of NOV1a transcript in these tissues may indicate that the gene product is not important for these cells.

In conclusion, taken together, the data from panels 1.2, 2D, and 3D indicate that this gene may play a role in cell proliferation. Therefore, inhibition of expression or function of this gene may be a therapeutic avenue for the treatment of cancer or other disease that involve cell proliferation.

Panel 4D Summary: The expression of the NOV1a transcript is limited to fibroblasts, endothelial cells, keratinocytes and CD45RA (naïve) T cells. In the fly, this transmembrane glycoprotein functions as a cell adhesion molecule and is involved in several important developmental processes including axonal pathfinding in the optic lobe, programmed cell death and pigment cell differentiation in the pupal retina (Ref. An Acad Bras Cienc. 2000 Sep;72(3):381-8. PMID: 11028102). Adhesion, apoptosis and differentiation of the cell types that express this molecule take place during immune responses and inflammation. Antibody therapeutics designed with the protein encoded for by this transcript could reduce or eliminate inflammation by blocking adhesion interactions between leukocytes and the endothelium. Likewise, an antagonistic soluble protein therapuetic would function by binding the ligand and prevent it from interacting with NOV1a. Protein and antibody therapeutics designed with the NOV1a protein could also block apoptosis induced by the activation of this molecule. These therapies could be important in the treatment of psoriasis, allergies, delayed type hypersensitivity, emphysema, and asthma.

NOV1B

Expression of the NOV1b gene was assessed using the primer-probe set Ag271, described in Table F. Results of the RTQ-PCR runs are shown in Table G.

TABLE F
Probe Name: Ag271

| | | | | |
|---|---|---|---|---|
| | | | | Start |
| Primers | Sequences | TM | Length | Position |

(SEQ ID NO:101)

| | | | |
|---|---|---|---|
| Forward | 5′-ACCTGGACATAGGGCGTGTC | 21 | 160 |

T-3′
(SEQ ID NO:102)

| | | | |
|---|---|---|---|
| Probe | FAM-5′-CGAAGCATGAACGAAGC | 26 | 189 |

CATCCCTAG-3′-TAMRA
(SEQ ID NO:103)

| | | | |
|---|---|---|---|
| Reverse | 5′-TCGATGGAAGTCTCCTTGCC- | 20 | 216 |

3′

TABLE G
Panel 1

| | Relative | Relative |
|---|---|---|
| | Expression (%) | Expression (%) |
| Tissue Name | 1xtm371f_ag271 | 1xtm418f_ag271 |
| Endothelial cells | 11.0 | 5.0 |
| Endothelial cells (treated) | 3.8 | 4.3 |
| Pancreas | 2.7 | 3.1 |
| Pancreatic ca. CAPAN 2 | 38.2 | 24.0 |
| Adipose | 92.0 | 39.8 |
| Adrenal gland | 14.1 | 8.2 |
| Thyroid | 8.6 | 6.1 |
| Salivary gland | 3.5 | 4.1 |
| Pituitary gland | 9.5 | 10.2 |
| Brain (fetal) | 7.4 | 9.0 |
| Brain (whole) | 0.8 | 3.7 |
| Brain (amygdala) | 0.1 | 1.7 |
| Brain (cerebellum) | 4.8 | 10.5 |
| Brain (hippocampus) | 0.2 | 1.2 |
| Brain (substantia nigra) | 0.9 | 3.5 |
| Brain (thalamus) | 0.7 | 2.7 |
| Brain (hypothalamus) | 0.5 | 3.4 |
| Spinal cord | 2.1 | 4.0 |
| CNS ca. (glio/astro) U87-MG | 30.8 | 24.0 |
| CNS ca. (glio/astro) U-118-MG | 28.5 | 24.8 |
| CNS ca. (astro) SW1783 | 21.0 | 17.1 |
| CNS ca.* (neuro; met) SK-N-AS | 24.1 | 17.6 |
| CNS ca. (astro) SF-539 | 35.6 | 27.4 |
| CNS ca. (astro) SNB-75 | 56.3 | 65.1 |
| CNS ca. (glio) SNB-19 | 42.6 | 53.6 |
| CNS ca. (glio) U251 | 39.8 | 26.8 |
| CNS ca. (glio) SF-295 | 43.2 | 33.4 |
| Heart | 15.2 | 4.5 |
| Skeletal muscle | 0.1 | 1.9 |
| Bone marrow | 1.1 | 1.7 |
| Thymus | 28.1 | 18.9 |
| Spleen | 2.5 | 5.1 |
| Lymph node | 6.8 | 6.0 |
| Colon (ascending) | 3.4 | 3.2 |
| Stomach | 11.6 | 12.0 |
| Small intestine | 5.9 | 8.7 |
| Colon ca. SW480 | 0.0 | 1.8 |
| Colon ca.* (SW480 met)SW620 | 1.5 | 2.4 |
| Colon ca. HT29 | 0.0 | 1.7 |
| Colon ca. HCT-116 | 13.0 | 10.4 |
| Colon ca. CaCo-2 | 27.9 | 21.8 |
| Colon ca. HCT-15 | 1.7 | 5.0 |
| Colon ca. HCC-2998 | 0.4 | 1.2 |
| Gastric ca.* (liver met) NCI-N87 | 1.1 | 3.1 |
| Bladder | 30.8 | 15.9 |
| Trachea | 7.2 | 7.0 |
| Kidney | 14.8 | 8.9 |
| Kidney (fetal) | 53.6 | 55.9 |
| Renal ca. 786-0 | 94.6 | 96.6 |
| Renal ca. A498 | 65.5 | 65.5 |
| Renal ca. RXF 393 | 41.8 | 27.7 |
| Renal ca. ACHN | 66.9 | 65.1 |
| Renal ca. UO-31 | 46.0 | 41.8 |
| Renal ca. TK-10 | 57.0 | 56.6 |
| Liver | 2.0 | 3.3 |
| Liver (fetal) | 2.0 | 2.4 |
| Liver ca. (hepatoblast) HepG2 | 0.0 | 0.0 |
| Lung | 27.9 | 8.0 |
| Lung (fetal) | 23.8 | 11.9 |
| Lung ca. (small cell) LX-1 | 0.0 | 1.4 |
| Lung ca. (small cell) NCI-H69 | 2.9 | 4.2 |
| Lung ca. (s. cell var.) SHP-77 | 0.0 | 0.4 |
| Lung ca. (large cell) NCI-H460 | 29.5 | 27.0 |
| Lung ca. (non-sm. cell) A549 | 6.6 | 7.1 |
| Lung ca. (non-s. cell) NCI-H23 | 6.6 | 7.1 |
| Lung ca (non-s. cell) HOP-62 | 19.1 | 15.8 |
| Lung ca. (non-s. cl) NCI-H522 | 4.3 | 5.4 |
| Lung ca. (squam.) SW 900 | 15.8 | 17.1 |
| Lung ca. (squam.) NCI-H596 | 8.3 | 8.7 |
| Mammary gland | 47.6 | 45.1 |
| Breast ca.* (pl. effusion) MCF-7 | 0.0 | 0.0 |
| Breast ca.* (pl. ef) MDA-MB-231 | 17.1 | 15.1 |
| Breast ca.* (pl. effusion) T47D | 3.1 | 5.3 |
| Breast ca. BT-549 | 70.7 | 65.1 |
| Breast ca. MDA-N | 43.8 | 25.7 |
| Ovary | 54.0 | 39.5 |
| Ovarian ca. OVCAR-3 | 32.8 | 32.3 |
| Ovarian ca. OVCAR-4 | 39.2 | 33.0 |
| Ovarian ca. OVCAR-5 | 49.3 | 35.6 |
| Ovarian ca. OVCAR-8 | 39.5 | 20.0 |
| Ovarian ca. IGROV-1 | 46.0 | 48.0 |
| Ovarian ca.* (ascites) SK-OV-3 | 53.2 | 47.6 |
| Uterus | 36.6 | 9.9 |
| Placenta | 18.2 | 23.8 |
| Prostate | 6.4 | 6.7 |
| Prostate ca.* (bone met)PC-3 | 32.8 | 37.6 |
| Testis | 27.7 | 23.8 |
| Melanoma Hs688(A).T | 21.5 | 23.0 |
| Melanoma* (met) Hs688(B).T | 25.3 | 25.3 |
| Melanoma UACC-62 | 28.1 | 23.0 |
| Melanoma M14 | 34.6 | 36.1 |
| Melanoma LOX IMVI | 100.0 | 100.0 |
| Melanoma* (met) SK-MEL-5 | 12.1 | 10.9 |
| Melanoma SK-MEL-28 | 53.6 | 79.0 |

Panel 1 Summary: Expression of NOV1b in this panel is highest in a number of cancer cell lines, including pancreatic cancer, prostate cancer, melanoma, renal carcinoma, and CNS cancers, when compared to normal controls. Expression of this gene is also detected in most normal tissues with the highest expression in adipose. The results obtained from the two separate RTQ-PCR experiments using Ag271 are roughly in agreement. The data presented in panel 1.2 reveal that this gene is expressed quite highly in samples derived from cancer cell lines and not expressed highly in samples derived from normal tissues. In addition, within the normal kidney samples there seems to be a consistent difference between adult and fetal tissues. Taking into account that cell lines are, on the whole, more proliferative than tissues and that fetal tissues also show a higher proliferative compartment than their adult counterparts, it is speculated that this gene might be involved in cell proliferation. Thus, taken together, the data from panels 1.2, 2D, and 3D indicate that this gene may play a role in cell proliferation. The NOV1b gene may also play a role in cell migration and invasion as well as metastatic potential. Therefore, inhibition of expression or function of this gene may be a therapeutic avenue for the treatment of cancer or other disease that involve cell proliferation. Furthermore, therapeutic targeting of NOV1b with a monoclonal antibody is anticipated to limit or block the extent of tumor cell migration and invasion and tumor metastasis, particularly in melanomas, prostate cancers, renal cell carcinomas and CNS cancers. Expression in adipose may suggest that this gene plays a role in normal metabolic function and may be an important target for the treatment of metabolic diseases, including obesity and diabetes.

NOV7A

Expression of NOV7a was assessed using the primer-probe set Ag271b, described in Table H. Results of the RTQ-PCR run are shown in Table I. Its expression was also assessed using the primer-probe sets Ag689 and Ag271 described in Tables A and F; the RTQ-PCR run results are presented in Tables B, C, D, E, and G and are summarized above.

TABLE H
Probe Name: Ag271b

| | | | | |
|---|---|---|---|---|
| | | | | Start |
| Primers | Sequences | TM | Length | Position |

(SEQ ID NO:104)

| | | | |
|---|---|---|---|
| Forward | 5′-CACCGTGAGCCAACTGCTTA | 21 | 128 |

T-3′
(SEQ ID NO:105)

| | | | |
|---|---|---|---|
| Probe | FAM-5′-AGACACGCCCTATGTC | 24 | 157 |

CAGGTCCG-3′-TAMRA
(SEQ ID NO:106)

| | | | |
|---|---|---|---|
| Reverse | 5′-TTCGTTCATGCTTCGGCAA- | 19 | 185 |

3′

TABLE I
Panel 1

| | Relative |
|---|---|
| | Expression (%) |
| Tissue Name | 1xtm494f_ag271b |
| Endothelial cells | 3.9 |
| Endothelial cells (treated) | 4.9 |
| Pancreas | 1.9 |
| Pancreatic ca. CAPAN 2 | 19.8 |
| Adipose | 20.9 |
| Adrenal gland | 12.7 |
| Thyroid | 4.5 |
| Salivary gland | 3.3 |
| Pituitary gland | 7.3 |
| Brain (fetal) | 5.3 |
| Brain (whole) | 1.3 |
| Brain (amygdala) | 1.7 |
| Brain (cerebellum) | 2.3 |
| Brain (hippocampus) | 1.4 |
| Brain (substantia nigra) | 2.1 |
| Brain (thalamus) | 3.2 |
| Brain (hypothalamus) | 3.3 |
| Spinal cord | 3.6 |
| CNS ca. (glio/astro) U87-MG | 27.4 |
| CNS ca. (glio/astro) U-118-MG | 20.0 |
| CNS ca. (astro) SW1783 | 17.0 |
| CNS ca.* (neuro; met) SK-N-AS | 29.9 |
| CNS ca. (astro) SF-539 | 41.2 |
| CNS ca. (astro) SNB-75 | 32.8 |
| CNS ca. (glio) SNB-19 | 55.9 |
| CNS ca. (glio) U251 | 48.3 |
| CNS ca. (glio) SF-295 | 31.9 |
| Heart | 6.8 |
| Skeletal muscle | 4.6 |
| Bone marrow | 2.1 |
| Thymus | 10.2 |
| Spleen | 3.2 |
| Lymph node | 2.0 |
| Colon (ascending) | 2.6 |
| Stomach | 3.3 |
| Small intestine | 5.5 |
| Colon ca. SW480 | 5.2 |
| Colon ca.* (SW480 met)SW620 | 3.3 |
| Colon ca. HT29 | 3.2 |
| Colon ca. HCT-116 | 8.9 |
| Colon ca. CaCo-2 | 17.3 |
| Colon ca. HCT-15 | 7.3 |
| Colon ca. HCC-2998 | 2.7 |
| Gastric ca.* (liver met) NCI-N87 | 3.5 |
| Bladder | 19.1 |
| Trachea | 4.4 |
| Kidney | 9.8 |
| Kidney (fetal) | 27.7 |
| Renal ca. 786-0 | 70.2 |
| Renal ca. A498 | 46.3 |
| Renal ca. RXF 393 | 29.1 |
| Renal ca. ACHN | 51.4 |
| Renal ca. UO-31 | 54.0 |
| Renal ca. TK-10 | 52.8 |
| Liver | 4.4 |
| Liver (fetal) | 2.9 |
| Liver ca. (hepatoblast) HepG2 | 0.0 |
| Lung | 5.0 |
| Lung (fetal) | 5.3 |
| Lung ca. (small cell) LX-1 | 3.2 |
| Lung ca. (small cell) NCI-H69 | 8.2 |
| Lung ca. (s. cell var.) SHP-77 | 0.2 |
| Lung ca. (large cell) NCI-H460 | 51.4 |
| Lung ca. (non-sm. cell) A549 | 12.2 |
| Lung ca. (non-s. cell) NCI-H23 | 17.2 |
| Lung ca (non-s. cell) HOP-62 | 40.9 |
| Lung ca. (non-s. cl) NCI-H522 | 5.0 |
| Lung ca. (squam.) SW 900 | 19.1 |
| Lung ca. (squam.) NCI-H596 | 10.5 |
| Mammary gland | 28.9 |
| Breast ca.* (pl. effusion) MCF-7 | 0.0 |
| Breast ca.* (pl. ef) MDA-MB-231 | 18.4 |
| Breast ca.* (pl. effusion) T47D | 6.4 |
| Breast ca. BT-549 | 26.1 |
| Breast ca. MDA-N | 35.4 |
| Ovary | 27.9 |
| Ovarian ca. OVCAR-3 | 55.5 |
| Ovarian ca. OVCAR-4 | 46.0 |
| Ovarian ca. OVCAR-5 | 46.7 |
| Ovarian ca. OVCAR-8 | 57.4 |
| Ovarian ca. IGROV-1 | 100.0 |
| Ovarian ca. (ascites) SK-OV-3 | 68.8 |
| Uterus | 9.4 |
| Placenta | 11.7 |
| Prostate | 4.3 |
| Prostate ca.* (bone met) PC-3 | 50.3 |
| Testis | 4.7 |
| Melanoma Hs688 (A).T | 13.6 |
| Melanoma* (met) Hs688 (B).T | 20.3 |
| Melanoma UACC-62 | 58.2 |
| Melanoma M14 | 22.2 |
| Melanoma LOX IMVI | 12.2 |
| Melanoma* (met) SK-MEL-5 | 14.3 |
| Melanoma SK-MEL-28 | 2.5 |

Panel 1 Summary: Expression of gene NOV7a in this panel is highest in a number of cancer cell lines, including ovarian cancer, pancreatic cancer, prostate cancer, melanoma, renal carcinoma, and CNS cancers, when compared to normal controls. Expression of this gene is also detected at lower levels in most normal tissues, with the highest expression in mammary gland. The data presented in panel 1 reveal that this gene is expressed quite highly in samples derived from cancer cell lines and not expressed highly in samples derived from normal tissues. In addition, within the normal kidney samples there seems to be a consistent difference between adult and fetal tissues. Taking into account that cell lines are, on the whole, more proliferative than tissues and that fetal tissues also show a higher proliferative compartment than their adult counterparts, it is speculated that this gene might be involved in cell proliferation. Therefore, inhibition of expression or function of this gene may be a therapeutic avenue for the treatment of cancer or other disease that involve cell proliferation. Furthermore, therapeutic targeting of NOV7a with a monoclonal antibody is anticipated to limit or block the extent of tumor cell migration and invasion and tumor metastasis, particularly in melanomas, prostate cancers, pancreatic cancers, ovarian cancers, renal cell carcinomas and CNS cancers. This gene might also be an effective marker for the diagnosis and detection of a variety of cancers.

NOV2

Expression of gene NOV2 was assessed using the primer-probe sets Ag995, Ag98, Ag883, and Ag2749 described in Tables J, K, L, and M. Results of the RTQ-PCR runs are shown in Table N, O, P, Q, R, and S.

TABLE J
Probe Name Ag995

| | | | | Start |
|---|---|---|---|---|
| Primers | Sequences | TM | Length | Position |
| Forward | 5′-GAGATGCAGTGCATGTG | 59.8 | 22 | 1274 |
| | TATGA-3′ (SEQ ID NO: 107) | | | |
| Probe | FAM-5′-CCTGAGAGCTCCCG | 69.2 | 24 | 1309 |
| | ACTACTGCGT-3′-TAMRA | | | |
| | (SEQ ID NO: 108) | | | |
| Reverse | 5′-TTGCTGAAGTGGGTGAG | 58.4 | 20 | 1333 |
| | ACT-3′ (SEQ ID NO: 109) | | | |

TABLE K
Probe Name Ag98

| | | | | Start |
|---|---|---|---|---|
| Primers | Sequences | TM | Length | Position |
| Forward | 5′-TGTCCCTGGATTTCA | | 21 | 2538 |
| | GGGACT-3′ (SEQ | | | |
| | ID NO: 110) | | | |
| Probe | CCTCCCGTTGACCCTATGTAG | | 33 | 2566 |
| | CTGCTATAAGTT-3′-TAMRA | | | |
| | (SEQ ID NO: 111) | | | |
| Reverse | 5′-TCCCTGCCTGAGGGA | | 19 | 2600 |
| | CACT-3′ (SEQ ID | | | |
| | NO: 112 | | | |

TABLE L
Probe Name Ag883

| | | | | Start |
|---|---|---|---|---|
| Primers | Sequences | TM | Length | Position |
| Forward | 5′-AGTGCAGGGCACC | 58.9 | 19 | 404 |
| | TTACAG-3′ (SEQ ID | | | |
| | NO: 113) | | | |
| Probe | FAM-5′-CAACTGCACCTGGCTC | 70.3 | 23 | 458 |
| | ATCCTGG-3′-TAMRA | | | |
| | (SEQ ID NO: 114) | | | |
| Reverse | 5′-GGTGACAGTCTGTTCC | 59.4 | 21 | 483 |
| | TTGCT-3′ (SEQ ID | | | |
| | NO: 115) | | | |

TABLE M
Probe Name Ag2749

| | | | | Start |
|---|---|---|---|---|
| Primers | Sequences | TM | Length | Position |
| Forward | 5′-AGATGCAGTGCATGTG | 58.7 | 22 | 152 |
| | TATGAC-3′ (SEQ ID | | | |
| | NO: 116) | | | |
| Probe | TET-5′-CCTGAGAGCTCCCGACT | 68.8 | 23 | 185 |
| | ACTGCG-3′-TAMRA (SEQ ID | | | |
| | NO: 117) | | | |
| Reverse | 5′-ATTGCTGAAGTGGGTGA | 58.9 | 22 | 208 |
| | GACTA-3′ (SEQ ID | | | |
| | NO: 118) | | | |

TABLE N
Panel 1

| | Relative | | Relative |
|---|---|---|---|
| | Expression | | Expression |
| | (%) | | (%) |
| Tissue Name | 1tm258f_ag98 | Tissue Name | 1tm258f_ag98 |
| Endothelial cells | 7.4 | Kidney (fetal) | 46.0 |
| Endothelial cells (treated) | 10.2 | Renal ca. 786-0 | 30.8 |
| Pancreas | 32.8 | Renal ca. A498 | 21.6 |
| Pancreatic ca. CAPAN 2 | 30.1 | Renal ca. RXF 393 | 3.8 |
| Adipose | 12.2 | Renal ca. ACHN | 16.6 |
| Adrenal gland | 55.5 | Renal ca. UO-31 | 4.4 |
| Thyroid | 60.7 | Renal ca. TK-10 | 15.4 |
| Salivary gland | 49.3 | Liver | 39.2 |
| Pituitary gland | 28.1 | Liver (fetal) | 17.8 |
| Brain (fetal) | 1.5 | Liver ca. (hepatoblast) HepG2 | 5.3 |
| Brain (whole) | 1.0 | Lung | 3.4 |
| Brain (amygdala) | 1.8 | Lung (fetal) | 21.2 |
| Brain (cerebellum) | 0.0 | Lung ca. (small cell) LX-1 | 5.1 |
| Brain (hippocampus) | 15.0 | Lung ca. (small cell) NCI-H69 | 1.4 |
| Brain (substantia nigra) | 9.5 | Lung ca. (s. cell var.) SHP-77 | 0.0 |
| Brain (thalamus) | 6.0 | Lung ca. (large cell) NCI-H460 | 0.0 |
| Brain (hypothalamus) | 25.9 | Lung ca. (non-sm. cell) A549 | 46.7 |
| Spinal cord | 27.9 | Lung ca. (non-s. cell) NCI-H23 | 33.9 |
| CNS ca. (glio/astro) U87-MG | 11.6 | Lung ca (non-s. cell) HOP-62 | 14.0 |
| CNS ca. (glio/astro) U-118-MG | 9.2 | Lung ca. (non-s. cl) NCI-H522 | 26.1 |
| CNS ca. (astro) SW1783 | 9.8 | Lung ca. (squam.) SW 900 | 34.2 |
| CNS ca.* (neuro; met) SK-N- | 6.7 | Lung ca. (squam.) NCI-H596 | 0.0 |
| AS | | | |
| CNS ca. (astro) SF-539 | 9.8 | Mammary gland | 44.8 |
| CNS ca. (astro) SNB-75 | 14.8 | Breast ca.* (pl. effusion) MCF- | 47.0 |
| | | 7 | |
| CNS ca. (glio) SNB-19 | 11.7 | Breast ca.* (pl. ef) MDA-MB- | 11.6 |
| | | 231 | |
| CNS ca. (glio) U251 | 3.9 | Breast ca.* (pl. effusion) T47D | 27.9 |
| CNS ca. (glio) SF-295 | 15.5 | Breast ca. BT-549 | 0.0 |
| Heart | 29.9 | Breast ca. MDA-N | 7.4 |
| Skeletal muscle | 6.5 | Ovary | 14.0 |
| Bone marrow | 17.8 | Ovarian ca. OVCAR-3 | 7.2 |
| Thymus | 30.8 | Ovarian ca. OVCAR-4 | 11.1 |
| Spleen | 23.7 | Ovarian ca. OVCAR-5 | 81.8 |
| Lymph node | 45.7 | Ovarian ca. OVCAR-8 | 24.1 |
| Colon (ascending) | 4.8 | Ovarian ca. IGROV-1 | 9.0 |
| Stomach | 25.7 | Ovarian ca.* (ascites) SK-OV- | 6.8 |
| | | 3 | |
| Small intestine | 35.6 | Uterus | 54.7 |
| Colon ca. SW480 | 2.7 | Placenta | 83.5 |
| Colon ca.* (SW480 | 3.5 | Prostate | 33.9 |
| met)SW620 | | | |
| Colon ca. HT29 | 8.5 | Prostate ca.* (bone met) PC-3 | 0.0 |
| Colon ca. HCT-116 | 0.0 | Testis | 21.8 |
| Colon ca. CaCo-2 | 11.5 | Melanoma Hs688(A).T | 14.1 |
| Colon ca. HCT-15 | 37.6 | Melanoma* (met) Hs688(B).T | 24.5 |
| Colon ca. HCC-2998 | 11.6 | Melanoma UACC-62 | 5.3 |
| Gastric ca.* (liver met) NCI- | 17.1 | Melanoma M14 | 6.4 |
| N87 | | | |
| Bladder | 6.1 | Melanoma LOX IMVI | 23.0 |
| Trachea | 100.0 | Melanoma* (met) SK-MEL-5 | 18.7 |
| Kidney | 56.6 | Melanoma SK-MEL-28 | 6.7 |

TABLE O
Panel 1.2

| | Relative | Relative | Relative |
|---|---|---|---|
| | Expression | Expression | Expression |
| | (%) | (%) | (%) |
| Tissue Name | 1.2tm1083f_ag995 | 1.2tm1346f_ag995 | 1.2tm998f_ag883 |
| Endothelial cells | 19.6 | 12.7 | 17.2 |
| Endothelial cells (treated) | 26.4 | 36.9 | 23.2 |
| Pancreas | 25.9 | 1.7 | 50.7 |
| Pancreatic ca. CAPAN 2 | 19.3 | 6.5 | 30.6 |
| Adrenal Gland (new lot*) | 25.5 | 15.9 | 24.3 |
| Thyroid | 66.4 | 11.2 | 52.1 |
| Salivary gland | 51.0 | 42.6 | 47.3 |
| Pituitary gland | 21.2 | 12.1 | 18.9 |
| Brain (fetal) | 5.4 | 0.7 | 5.3 |
| Brain (whole) | 11.4 | 3.5 | 8.0 |
| Brain (amygdala) | 6.5 | 4.7 | 5.7 |
| Brain (cerebellum) | 3.3 | 1.4 | 3.5 |
| Brain (hippocampus) | 9.6 | 8.4 | 8.1 |
| Brain (thalamus) | 6.2 | 2.0 | 5.5 |
| Cerebral Cortex | 10.1 | 14.6 | 10.7 |
| Spinal cord | 15.6 | 9.2 | 13.0 |
| CNS ca. (glio/astro) U87-MG | 22.4 | 4.6 | 18.0 |
| CNS ca. (glio/astro) U-118-MG | 13.1 | 6.2 | 13.8 |
| CNS ca. (astro) SW1783 | 16.2 | 9.5 | 13.0 |
| CNS ca.* (neuro; met) SK-N-AS | 23.0 | 6.8 | 18.4 |
| CNS ca. (astro) SF-539 | 24.1 | 3.2 | 15.9 |
| CNS ca. (astro) SNB-75 | 4.5 | 4.2 | 6.7 |
| CNS ca. (glio) SNB-19 | 28.7 | 9.2 | 11.5 |
| CNS ca. (glio) U251 | 10.2 | 3.3 | 8.4 |
| CNS ca. (glio) SF-295 | 27.4 | 6.4 | 21.6 |
| Heart | 36.3 | 59.0 | 35.1 |
| Skeletal Muscle (new lot*) | 59.5 | 11.0 | 31.2 |
| Bone marrow | 10.7 | 4.0 | 13.3 |
| Thymus | 6.9 | 2.1 | 7.1 |
| Spleen | 15.6 | 5.2 | 12.2 |
| Lymph node | 14.8 | 10.3 | 14.9 |
| Colorectal | 4.4 | 17.0 | 4.3 |
| Stomach | 36.1 | 33.9 | 40.9 |
| Small intestine | 37.6 | 8.8 | 28.1 |
| Colon ca. SW480 | 4.4 | 1.4 | 3.1 |
| Colon ca.* (SW480 met)SW620 | 8.2 | 1.3 | 4.4 |
| Colon ca. HT29 | 12.2 | 1.6 | 8.6 |
| Colon ca. HCT-116 | 7.1 | 2.7 | 6.0 |
| Colon ca. CaCo-2 | 16.5 | 8.4 | 8.5 |
| 83219 CC Well to Mod Diff (ODO3866) | 10.4 | 8.4 | 3.2 |
| Colon ca. HCC-2998 | 46.7 | 13.1 | 25.2 |
| Gastric ca.* (liver met) NCI-N87 | 28.7 | 10.9 | 19.3 |
| Bladder | 44.4 | 28.1 | 56.3 |
| Trachea | 19.8 | 14.6 | 19.6 |
| Kidney | 43.5 | 7.7 | 33.0 |
| Kidney (fetal) | 26.8 | 24.5 | 25.3 |
| Renal ca. 786-0 | 23.2 | 15.0 | 10.7 |
| Renal ca. A498 | 41.2 | 13.9 | 35.6 |
| Renal ca. RXF 393 | 4.0 | 3.3 | 3.4 |
| Renal ca. ACHN | 21.0 | 6.3 | 14.4 |
| Renal ca. UO-31 | 19.1 | 4.1 | 9.0 |
| Renal ca. TK-10 | 18.0 | 12.3 | 10.6 |
| Liver | 15.6 | 8.9 | 12.9 |
| Liver (fetal) | 13.5 | 8.1 | 18.0 |
| Liver ca. (hepatoblast) HepG2 | 3.3 | 7.6 | 3.2 |
| Lung | 21.2 | 21.3 | 22.4 |
| Lung (fetal) | 13.6 | 6.1 | 10.4 |
| Lung ca. (small cell) LX-1 | 11.3 | 2.9 | 14.6 |
| Lung ca. (small cell) NCI-H69 | 2.1 | 2.1 | 2.2 |
| Lung ca. (s. cell var.) SHP-77 | 2.8 | 2.1 | 2.6 |
| Lung ca. (large cell) NCI-H460 | 59.9 | 100.0 | 65.5 |
| Lung ca. (non-sm. cell) A549 | 55.5 | 29.7 | 48.3 |
| Lung ca. (non-s. cell) NCI-H23 | 38.2 | 30.1 | 27.0 |
| Lung ca (non-s. cell) HOP-62 | 38.4 | 8.1 | 37.9 |
| Lung ca. (non-s. cl) NCI-H522 | 36.9 | 12.1 | 40.1 |
| Lung ca. (squam.) SW 900 | 27.9 | 14.8 | 14.5 |
| Lung ca. (squam.) NCI-H596 | 1.1 | 0.2 | 0.8 |
| Mammary gland | 44.4 | 12.3 | 43.2 |
| Breast ca.* (pl. effusion) MCF-7 | 35.6 | 39.5 | 33.0 |
| Breast ca.* (pl. ef) MDA-MB-231 | 20.3 | 2.9 | 12.9 |
| Breast ca.* (pl. effusion) T47D | 35.4 | 5.4 | 26.4 |
| Breast ca. BT-549 | 20.3 | 13.1 | 23.7 |
| Breast ca. MDA-N | 7.4 | 3.7 | 6.2 |
| Ovary | 22.8 | 33.4 | 27.2 |
| Ovarian ca. OVCAR-3 | 20.6 | 4.0 | 18.6 |
| Ovarian ca. OVCAR-4 | 19.2 | 6.6 | 20.0 |
| Ovarian ca. OVCAR-5 | 100.0 | 82.4 | 100.0 |
| Ovarian ca. OVCAR-8 | 22.2 | 17.7 | 13.4 |
| Ovarian ca. IGROV-1 | 26.6 | 12.9 | 28.1 |
| Ovarian ca.* (ascites) SK-OV-3 | 27.7 | 15.1 | 24.3 |
| Uterus | 21.3 | 11.7 | 23.3 |
| Placenta | 43.8 | 16.4 | 45.7 |
| Prostate | 31.6 | 74.7 | 27.2 |
| Prostate ca* (bone met)PC-3 | 92.7 | 58.6 | 94.6 |
| Testis | 16.0 | 7.0 | 11.7 |
| Melanoma Hs688(A).T | 15.6 | 6.4 | 15.0 |
| Melanoma* (met) Hs688(B).T | 21.0 | 12.0 | 20.9 |
| Melanoma UACC-62 | 23.7 | 11.4 | 23.0 |
| Melanoma M14 | 4.9 | 2.7 | 9.0 |
| Melanoma LOX IMVI | 9.8 | 3.8 | 7.6 |
| Melanoma* (met) SK-MEL-5 | 13.9 | 6.0 | 16.2 |
| Adipose | 2.5 | 14.6 | 2.5 |

TABLE P
Panel 1.3D

| | Relative | Relative |
|---|---|---|
| | Expression | Expression |
| | (%) | (%) |
| Tissue Name | 1.3dtm3949f_ag995 | 1.3Dtm4854t_ag2749_b1 |
| Liver adenocarcinoma | 44.4 | 38.3 |
| Pancreas | 10.4 | 2.6 |
| Pancreatic ca. CAPAN 2 | 33.7 | 17.8 |
| Adrenal gland | 14.7 | 5.4 |
| Thyroid | 42.0 | 12.8 |
| Salivary gland | 8.4 | 6.6 |
| Pituitary gland | 11.2 | 2.9 |
| Brain (fetal) | 1.6 | 0.9 |
| Brain (whole) | 2.2 | 4.1 |
| Brain (amygdala) | 11.1 | 6.0 |
| Brain (cerebellum) | 1.2 | 2.6 |
| Brain (hippocampus) | 29.1 | 7.8 |
| Brain (substantia nigra) | 1.1 | 1.7 |
| Brain (thalamus) | 6.9 | 3.4 |
| Cerebral Cortex | 3.0 | 18.7 |
| Spinal cord | 6.7 | 18.8 |
| CNS ca. (glio/astro) U87-MG | 17.4 | 21.4 |
| CNS ca. (glio/astro) U-118-MG | 32.5 | 10.1 |
| CNS ca. (astro) SW1783 | 25.2 | 47.3 |
| CNS ca.* (neuro; met) SK-N-AS | 12.1 | 4.4 |
| CNS ca. (astro) SF-539 | 10.8 | 19.3 |
| CNS ca. (astro) SNB-75 | 21.6 | 15.1 |
| CNS ca. (glio) SNB-19 | 6.8 | 15.4 |
| CNS ca. (glio) U251 | 4.0 | 5.5 |
| CNS ca. (glio) SF-295 | 34.9 | 22.1 |
| Heart (fetal) | 36.1 | 43.0 |
| Heart | 7.3 | 8.9 |
| Fetal Skeletal | 100.0 | 100.0 |
| Skeletal muscle | 1.7 | 9.6 |
| Bone marrow | 25.5 | 8.8 |
| Thymus | 12.1 | 30.2 |
| Spleen | 20.3 | 14.5 |
| Lymph node | 4.4 | 5.0 |
| Colorectal | 17.4 | 20.6 |
| Stomach | 14.8 | 9.2 |
| Small intestine | 17.0 | 13.8 |
| Colon ca. SW480 | 10.1 | 3.8 |
| Colon ca. (SW480 met)SW620 | 5.4 | 2.7 |
| Colon ca. HT29 | 11.7 | 7.1 |
| Colon ca. HCT-116 | 5.2 | 2.6 |
| Colon ca. CaCo-2 | 8.7 | 10.3 |
| 83219 CC Well to Mod Diff (ODO3866) | 30.4 | 33.3 |
| Colon ca. HCC-2998 | 18.2 | 7.2 |
| Gastric ca.* (liver met) NCI-N87 | 15.9 | 10.1 |
| Bladder | 9.5 | 18.6 |
| Trachea | 60.3 | 35.2 |
| Kidney | 9.3 | 21.9 |
| Kidney (fetal) | 16.7 | 21.3 |
| Renal ca. 786-0 | 7.4 | 9.5 |
| Renal ca. A498 | 39.5 | 26.1 |
| Renal ca. RXF 393 | 2.3 | 7.5 |
| Renal ca. ACHN | 10.8 | 10.3 |
| Renal ca. UO-31 | 25.2 | 13.2 |
| Renal ca. TK-10 | 10.2 | 3.6 |
| Liver | 2.0 | 1.2 |
| Liver (fetal) | 12.2 | 9.1 |
| Liver ca. (hepatoblast) HepG2 | 5.9 | 2.3 |
| Lung | 2.0 | 1.2 |
| Lung (fetal) | 6.5 | 9.8 |
| Lung ca. (small cell) LX-1 | 7.8 | 4.5 |
| Lung ca. (small cell) NCI-H69 | 5.3 | 1.7 |
| Lung ca. (s. cell var.) SHP-77 | 9.4 | 5.3 |
| Lung ca. (large cell) NCI-H460 | 16.4 | 18.9 |
| Lung ca. (non-sm. cell) A549 | 26.2 | 10.2 |
| Lung ca. (non-s. cell) NCI-H23 | 38.7 | 37.7 |
| Lung ca (non-s. cell) HOP-62 | 20.3 | 18.4 |
| Lung ca. (non-s. cl) NCI-H522 | 12.1 | 5.5 |
| Lung ca. (squam.) SW 900 | 9.2 | 8.1 |
| Lung ca. (squam.) NCI-H596 | 0.2 | 0.3 |
| Mammary gland | 14.4 | 15.9 |
| Breast ca.* (pl. effusion) MCF-7 | 18.6 | 38.2 |
| Breast ca.* (pl. ef) MDA-MB-231 | 50.2 | 11.5 |
| Breast ca.* (pl. effusion) T47D | 5.1 | 9.7 |
| Breast ca. BT-549 | 36.1 | 11.8 |
| Breast ca. MDA-N | 1.2 | 2.7 |
| Ovary | 62.4 | 73.8 |
| Ovarian ca. OVCAR-3 | 2.6 | 4.6 |
| Ovarian ca. OVCAR-4 | 7.9 | 2.8 |
| Ovarian ca. OVCAR-5 | 72.2 | 29.8 |
| Ovarian ca. OVCAR-8 | 30.6 | 15.3 |
| Ovarian ca. IGROV-1 | 13.1 | 4.6 |
| Ovarian ca.* (ascites) SK-OV-3 | 7.2 | 7.2 |
| Uterus | 24.3 | 9.9 |
| Placenta | 34.6 | 16.6 |
| Prostate | 16.6 | 9.5 |
| Prostate ca.* (bone met)PC-3 | 56.6 | 26.1 |
| Testis | 11.7 | 7.1 |
| Melanoma Hs688(A).T | 36.3 | 27.6 |
| Melanoma* (met) Hs688(B).T | 28.1 | 35.4 |
| Melanoma UACC-62 | 4.0 | 5.3 |
| Melanoma M14 | 4.0 | 2.4 |
| Melanoma LOX IMVI | 9.2 | 3.6 |
| Melanoma* (met) SK-MEL-5 | 8.6 | 5.2 |
| Adipose | 15.4 | 11.1 |

TABLE Q
Panel 2D

| | Relative | Relative |
|---|---|---|
| | Expression | Expression |
| | (%) | (%) |
| Tissue Name | 2dtm3950f_ag995 | 2dx4tm4644t_ag2749_b2 |
| Normal Colon GENPAK 061003 | 88.9 | 71.2 |
| 83219 CC Well to Mod Diff (ODO3866) | 40.3 | 27.7 |
| 83220 CC NAT (ODO3866) | 39.8 | 27.1 |
| 83221 CC Gr.2 rectosigmoid (ODO3868) | 20.7 | 13.5 |
| 83222 CC NAT (ODO3868) | 11.7 | 6.2 |
| 83235 CC Mod Diff (ODO3920) | 14.7 | 8.1 |
| 83236 CC NAT (ODO3920) | 33.9 | 25.3 |
| 83237 CC Gr.2 ascend colon (ODO3921) | 76.3 | 52.0 |
| 83238 CC NAT (ODO3921) | 36.9 | 24.1 |
| 83241 CC from Partial Hepatectomy (ODO4309) | 58.6 | 44.4 |
| 83242 Liver NAT (ODO4309) | 12.7 | 10.5 |
| 87472 Colon mets to lung (OD04451-01) | 28.9 | 19.6 |
| 87473 Lung NAT (OD04451-02) | 24.5 | 11.8 |
| Normal Prostate Clontech A+ 6546-1 | 27.2 | 100.0 |
| 84140 Prostate Cancer (OD04410) | 51.8 | 32.4 |
| 84141 Prostate NAT (OD04410) | 45.4 | 28.0 |
| 87073 Prostate Cancer (OD04720-01) | 25.5 | 17.2 |
| 87074 Prostate NAT (OD04720-02) | 55.1 | 40.8 |
| Normal Lung GENPAK 061010 | 48.0 | 38.2 |
| 83239 Lung Met to Muscle (ODO4286) | 47.0 | 37.5 |
| 83240 Muscle NAT (ODO4286) | 16.2 | 12.4 |
| 84136 Lung Malignant Cancer (ODO3126) | 75.8 | 62.2 |
| 84137 Lung NAT (OD03126) | 54.0 | 37.7 |
| 84871 Lung Cancer (OD04404) | 53.6 | 31.1 |
| 84872 Lung NAT (OD04404) | 40.6 | 26.5 |
| 84875 Lting Cancer (OD04565) | 20.6 | 13.9 |
| 84876 Lung NAT (OD04565) | 18.9 | 12.8 |
| 85950 Lung Cancer (OD04237-01) | 22.2 | 15.6 |
| 85970 Lung NAT (OD04237-02) | 33.9 | 26.7 |
| 83255 Ocular Mel Met to Liver (ODO4310) | 17.1 | 12.5 |
| 83256 Liver NAT (ODO4310) | 12.9 | 7.1 |
| 84139 Melanoma Mets to Lung (ODO4321) | 20.0 | 14.1 |
| 84138 Lung NAT (ODO4321) | 63.3 | 32.6 |
| Normal Kidney GENPAK 061008 | 53.2 | 43.6 |
| 83786 Kidney Ca, Nuclear grade 2 (OD04338) | 79.6 | 57.6 |
| 83787 Kidney NAT (OD04338) | 36.9 | 26.3 |
| 83788 Kidney Ca Nuclear grade ½ (OD04339) | 40.6 | 28.2 |
| 83789 Kidney NAT (OD04339) | 52.8 | 41.5 |
| 83790 Kidney Ca, Clear cell type (OD04340) | 50.7 | 41.6 |
| 83791 Kidney NAT (OD04340) | 41.5 | 31.1 |
| 83792 Kidney Ca, Nuclear grade 3 (OD04348) | 18.8 | 13.6 |
| 83793 Kidney NAT (OD04348) | 30.4 | 28.0 |
| 87474 Kidney Cancer (OD04622-01) | 41.8 | 28.5 |
| 87475 Kidney NAT (OD04622-03) | 17.6 | 14.3 |
| 85973 Kidney Cancer (OD04450-01) | 39.8 | 31.6 |
| 85974 Kidney NAT (OD04450-03) | 28.7 | 26.5 |
| Kidney Cancer Clontech 8120607 | 71.7 | 50.3 |
| Kidney NAT Clontech 8120608 | 61.1 | 39.6 |
| Kidney Cancer Clontech 8120613 | 79.0 | 53.3 |
| Kidney NAT Clontech 8120614 | 62.4 | 38.3 |
| Kidney Cancer Clontech 9010320 | 45.7 | 28.0 |
| Kidney NAT Clontech 9010321 | 100.0 | 78.0 |
| Normal Uterus GENPAK 061018 | 14.4 | 11.2 |
| Uterus Cancer GENPAK 064011 | 40.6 | 29.5 |
| Normal Thyroid Clontech A+ 6570-1 | 36.9 | 27.2 |
| Thyroid Cancer GENPAK 064010 | 53.6 | 43.4 |
| Thyroid Cancer INVITROGEN A302152 | 23.0 | 18.8 |
| Thyroid NAT INVITROGEN A302153 | 35.1 | 26.1 |
| Normal Breast GENPAK 061019 | 39.2 | 29.7 |
| 84877 Breast Cancer (OD04566) | 27.7 | 21.3 |
| 85975 Breast Cancer (OD04590-01) | 50.0 | 30.6 |
| 85976 Breast Cancer Mets (OD04590-03) | 54.3 | 43.9 |
| 87070 Breast Cancer Metastasis (OD04655-05) | 50.7 | 35.6 |
| GENPAK Breast Cancer 064006 | 23.2 | 17.5 |
| Breast Cancer Res. Gen. 1024 | 56.6 | 38.0 |
| Breast Cancer Clontech 9100266 | 50.0 | 41.7 |
| Breast NAT Clontech 9100265 | 27.0 | 26.0 |
| Breast Cancer INVITROGEN A209073 | 74.2 | 28.7 |
| Breast NAT INVITROGEN A2090734 | 26.6 | 16.8 |
| Normal Liver GENPAK 061009 | 7.3 | 5.4 |
| Liver Cancer GENPAK 064003 | 7.7 | 5.2 |
| Liver Cancer Research Genetics RNA 1025 | 6.9 | 5.0 |
| Liver Cancer Research Genetics RNA 1026 | 35.1 | 25.3 |
| Paired Liver Cancer Tissue Research Genetics RNA 6004-T | 13.3 | 7.2 |
| Paired Liver Tissue Research Genetics RNA 6004-N | 6.0 | 4.2 |
| Paired Liver Cancer Tissue Research Genetics RNA 6005-T | 34.9 | 25.2 |
| Paired Liver Tissue Research Genetics RNA 6005-N | 11.4 | 5.6 |
| Normal Bladder GENPAK 061001 | 54.0 | 38.3 |
| Bladder Cancer Research Genetics RNA 1023 | 39.2 | 26.2 |
| Bladder Cancer INVITROGEN A302173 | 17.1 | 8.7 |
| 87071 Bladder Cancer (OD04718-01) | 52.5 | 31.0 |
| 87072 Bladder Normal Adjacent (OD04718-03) | 45.7 | 28.0 |
| Normal Ovary Res. Gen. | 43.5 | 28.6 |
| Ovarian Cancer GENPAK 064008 | 46.3 | 27.7 |
| 87492 Ovary Cancer (OD04768-07) | 89.5 | 79.2 |
| 87493 Ovary NAT (OD04768-08) | 17.0 | 9.6 |
| Normal Stomach GENPAK 061017 | 39.2 | 29.9 |
| Gastric Cancer Clontech 9060358 | 13.1 | 8.0 |
| NAT Stomach Clontech 9060394 | 47.3 | 35.4 |
| Gastric Cancer Clontech 9060397 | 90.8 | 72.0 |
| NAT Stomach Clontech 9060396 | 49.7 | 34.5 |
| Gastric Cancer GENPAK 064005 | 43.2 | 34.5 |

TABLE R
Panel 3D

| | Relative |
|---|---|
| | Expression (%) |
| Tissue Name | 3dtm3951f_ag995 |
| 94905_Daoy_Medulloblastoma/ | 7.8 |
| Cerebellum_sscDNA | |
| 94906_TE671_Medulloblastom/ | 2.3 |
| Cerebellum_sscDNA | |
| 94907_D283 | 21.3 |
| Med_Medulloblastoma/Cerebell | |
| um_sscDNA | |
| 94908_PFSK-1_Primitive | 9.9 |
| Neuroectodermal/Cerebellum_s | |
| scDNA | |
| 94909_XF-498_CNS_sscDNA | 40.3 |
| 94910_SNB- | 45.4 |
| 78_CNS/glioma_sscDNA | |
| 94911_SF- | 9.5 |
| 268_CNS/glioblastoma_sscDN | |
| A | |
| 94912_T98G Glioblastoma_ssc | 16.5 |
| DNA | |
| 96776_SK-N- | 39.5 |
| SH_Neuroblastoma | |
| (metastasis)_sscDNA | |
| 94913_SF- | 17.3 |
| 295_CNS/glioblastoma_sscDN | |
| A | |
| 94914_Cerebellum_sscDNA | 5.6 |
| 96777_Cerebellum_sscDNA | 8.8 |
| 94916_NCI- | 80.7 |
| H292_Mucoepidermoid lung | |
| carcinoma_sscDNA | |
| 94917_DMS-114_Small cell | 5.6 |
| lung cancer_sscDNA | |
| 94918_DMS-79_Small cell | 34.9 |
| lung | |
| cancer/neuroendocrine_sscDNA | |
| 94919_NCI-H146_Small cell | 3.8 |
| lung | |
| cancer/neuroendocrine_sscDNA | |
| 94920_NCI-H526_Small cell | 4.3 |
| lung | |
| cancer/neuroendocrine_sscDNA | |
| 94921_NCI-N417_Small cell | 2.0 |
| lung | |
| cancer/neuroendocrine_sscDNA | |
| 94923_NCI-H82_Small cell | 1.0 |
| lung | |
| cancer/neuroendocrine_sscDNA | |
| 94924_NCI-H157_Squamous | 36.3 |
| cell lung cancer | |
| (metastasis)_sscDNA | |
| 94925_NCI-H1155_Large cell | 24.5 |
| lung | |
| cancer/neuroendocrine_sscDNA | |
| 94926_NCI-H1299_Large cell | 24.7 |
| lung | |
| cancer/neuroendocrine_sscDNA | |
| 94927_NCI-H727_Lung | 26.6 |
| carcinoid_sscDNA | |
| 94928_NCI-UMC-11_Lung | 33.7 |
| carcinoid_sscDNA | |
| 94929_LX-1_Small cell lung | 12.2 |
| cancer_sscDNA | |
| 94930_Colo-205_Colon | 27.0 |
| cancer_sscDNA | |
| 94931_KM12_Colon | 22.8 |
| cancer_sscDNA | |
| 94932_KM20L2_Colon | 10.4 |
| cancer_sscDNA | |
| 94933_NCI-H716_Colon | 54.3 |
| cancer_sscDNA | |
| 94935_SW-48_Colon | 22.5 |
| adenocarcinoma_sscDNA | |
| 94936_SW1116_Colon | 10.8 |
| adenocarcinoma_sscDNA | |
| 94937_LS 174T_Colon | 43.8 |
| adenocarcinoma_sscDNA | |
| 94938_SW-948_Colon | 1.7 |
| adenocarcinoma_sscDNA | |
| 94939_SW-480_Colon | 12.8 |
| adenocarcinoma_sscDNA | |
| 94940_NCI-SNU-5_Gastric | 12.1 |
| carcinoma_sscDNA | |
| 94941_KATO III_Gastric | 53.2 |
| carcinoma_sscDNA | |
| 94943_NCI-SNU-16_Gastric | 38.7 |
| carcinoma_sscDNA | |
| 94944_NCI-SNU-1_Gastric | 46.3 |
| carcinoma_sscDNA | |
| 94946_RF-1_Gastric | 15.5 |
| adenocarcinoma_sscDNA | |
| 94947_RF-48_Gastric | 12.2 |
| adenocarcinoma_sscDNA | |
| 96778_MKN-45_Gastric | 59.5 |
| carcinoma_sscDNA | |
| 94949_NCI-N87_Gastric | 6.6 |
| carcinoma_sscDNA | |
| 94951_OVCAR-5_Ovarian | 10.9 |
| carcinoma_sscDNA | |
| 94952_RL9S-2_Uterine | 15.9 |
| carcinoma_sscDNA | |
| 94953_HelaS3_Cervical | 11.7 |
| adenocarcinoma_sscDNA | |
| 94954_Ca Ski_Cervical | 29.7 |
| epidermoid carcinoma | |
| (metastasis)_sscDNA | |
| 94955_ES-2_Ovarian clear cell | 14.6 |
| carcinoma_sscDNA | |
| 94957_Ramos/6h stim_″; | 28.7 |
| Stimulated with | |
| PMA/ionomycin 6h_sscDNA | |
| 94958_Ramos/14h stim_″; | 32.8 |
| Stimulated with | |
| PMA/ionomycin 14h_sscDNA | |
| 94962_MEG-01_Chronic | 40.1 |
| myelogenous leukemia | |
| (megokaryoblast)_sscDNA | |
| 94963_Raji_Burkitt's | 2.0 |
| lymphoma_sscDNA | |
| 94964_Daudi_Burkitt's | 14.0 |
| lymphoma_sscDNA | |
| 94965_U266_B-cell | 15.7 |
| plasmacytoma/myeloma_sscDNA | |
| 94968_CA46_Burkitt's | 3.2 |
| lymphoma_sscDNA | |
| 94970_RL_non-Hodgkin's B- | 1.6 |
| cell lymphoma_sscDNA | |
| 94972_JM1_pre-B-cell | 4.6 |
| lymphoma/ | |
| leukemia_sscDNA | |
| 94973_Jurkat_T cell | 7.6 |
| leukemia_sscDNA | |
| 94974_TF- | 5.3 |
| 1_Erythroleukemia_sscDNA | |
| 94975_HUT 78_T-cell | 7.9 |
| lymphoma_sscDNA | |
| 94977_U937_Histiocytic | 3.9 |
| lymphoma_sscDNA | |
| 94980_KU-812_Myelogenous | 14.0 |
| leukemia_sscDNA | |
| 94981_769-P_Clear cell renal | 14.7 |
| carcinoma_sscDNA | |
| 94983_Caki-2_Clear cell renal | 23.0 |
| carcinoma_sscDNA | |
| 94984_SW 839_Clear cell renal | 48.0 |
| carcinoma_sscDNA | |
| 94986_G401_Wilms' | 7.5 |
| tumor_sscDNA | |
| 94987_Hs766T_Pancreatic | 41.5 |
| carcinoma (LN | |
| metastasis)_sscDNA | |
| 94988_CAPAN-l_Pancreatic | 30.6 |
| adenocarcinoma (liver | |
| metastasis)_sscDNA | |
| 94989_SU86.86_Pancreatic | 30.1 |
| carcinoma (liver | |
| metastasis)_sscDNA | |
| 94990_BxPC-3_Pancreatic | 30.4 |
| adenocarcinoma_sscDNA | |
| 94991_HPAC_Pancreatic | 66.4 |
| adenocarcinoma_sscDNA | |
| 94992_MIA PaCa-2_Pancreatic | 3.9 |
| carcinoma_sscDNA | |
| 94993_CFPAC-1_Pancreatic | 100.0 |
| ductal | |
| adenocarcinoma_sscDNA | |
| 94994_PANC-1_Pancreatic | 29.5 |
| epithelioid ductal | |
| carcinoma_sscDNA | |
| 94996_T24_Bladder carcinma | 28.9 |
| (transitional cell)_sscDNA | |
| 94997_5637_Bladder | 17.1 |
| carcinoma_sscDNA | |
| 94998_HT-1197_Bladder | 50.7 |
| carcinoma_sscDNA | |
| 94999_UM-UC-3_Bladder | 7.2 |
| carcinma (transitional | |
| cell)_sscDNA | |
| 95000_A204_Rhabdomyosarco | 17.6 |
| ma_sscDNA | |
| 95001_HT- | 59.0 |
| 1080_Fibrosarcoma_sscDNA | |
| 95002_MG-63_Osteosarcoma | 8.2 |
| (bone)_sscDNA | |
| 95003_SK-LMS- | 46.7 |
| 1_Leiomyosarcoma | |
| (vulva)_sscDNA | |
| 95004_SJRH30_Rhabdomyosar | 4.5 |
| coma (met to bone | |
| marrow)_sscDNA | |
| 95005_A431_Epidermoid | 43.5 |
| carcinoma_sscDNA | |
| 95007_WM266- | 11.9 |
| 4_Melanoma_sscDNA | |
| 95010_DU 145_Prostate | 0.7 |
| carcinoma | |
| (brain metastasis)_sscDNA | |
| 95012_MDA-MB-468_Breast | 7.0 |
| adenocarcinoma_sscDNA | |
| 95013_SCC-4 Squamous cell | 1.0 |
| carcinoma of tongue_sscDNA | |
| 95014_SCC-9_Squamous cell | 1.1 |
| carcinoma of tongue_sscDNA | |
| 95015_SCC-15_Squamous cell | 1.0 |
| carcinoma of tongue_sscDNA | |
| 95017_CAL 27_Squamous cell | 12.8 |
| carcinoma of tongue_sscDNA | |

TABLE S
Panel 4D

| | Relative | Relative |
|---|---|---|
| | Expression (%) | Expression (%) |
| Tissue Name | 4dtm3945f_ag995 | 4dx4tm4520t_ag2749_a2 |
| 93768_Secondary Th1_anti-CD28/anti-CD3 | 26.2 | 6.8 |
| 93769_Secondary Th2_anti-CD28/anti-CD3 | 9.0 | 6.9 |
| 93770_Secondary Tr1_anti-CD28/anti-CD3 | 27.2 | 6.0 |
| 93573_Secondary Th1_resting day 4-6 in IL-2 | 24.3 | 4.8 |
| 93572_Secondary Th2_resting day 4-6 in IL-2 | 20.0 | 6.7 |
| 93571_Secondary Tr1_resting day 4-6 in IL-2 | 21.5 | 7.0 |
| 93568_primary Th1_anti-CD28/anti-CD3 | 12.1 | 3.8 |
| 93569_primary Th2_anti-CD28/anti-CD3 | 12.7 | 4.8 |
| 93570_primary Tr1_anti-CD28/anti-CD3 | 20.3 | 5.3 |
| 93565_primary Th1_resting dy 4-6 in IL-2 | 56.3 | 16.0 |
| 93566_primary Th2_resting dy 4-6 in lL-2 | 35.8 | 9.4 |
| 93567_primary Tr1_resting dy 4-6 in IL-2 | 20.4 | 5.6 |
| 93351_CD45RA CD4 lymphocyte_anti-CD28/anti-CD3 | 35.6 | 8.2 |
| 93352_CD45RO CD4 lymphocyte_anti-CD28/anti-CD3 | 25.3 | 7.3 |
| 93251_CD8 Lymphocytes_anti-CD28/anti-CD3 | 11.9 | 3.2 |
| 93353_chronic CD8 Lymphocytes 2ry_resting dy 4-6 in IL-2 | 18.9 | 9.1 |
| 93574_chronic CD8 Lymphocytes 2ry_activated CD3/CD28 | 12.1 | 3.4 |
| 93354_CD4_none | 10.2 | 3.2 |
| 93252_Secondary Th1/Th2/Tr1_anti-CD95 CH11 | 22.4 | 9.2 |
| 93103_LAK cells_resting | 32.1 | 10.9 |
| 93788_LAK cells_IL-2 | 30.4 | 8.3 |
| 93787_LAK cells_IL-2 + IL-12 | 17.7 | 5.2 |
| 93789_LAK cells_IL-2 + IFN gamma | 29.5 | 8.6 |
| 93790_LAK cells_IL-2 + IL-18 | 29.7 | 6.0 |
| 93104_LAK cells_PMA/ionomycin and IL-18 | 20.6 | 5.0 |
| 93578_NK Cells IL-2_resting | 26.4 | 6.6 |
| 93109_Mixed Lymphocyte Reaction_Two Way MLR | 29.7 | 10.3 |
| 93110_Mixed Lymphocyte Reaction_Two Way MLR | 18.3 | 5.7 |
| 93111_Mixed Lymphocyte Reaction_Two Way MLR | 15.2 | 5.6 |
| 93112_Mononuclear Cells (PBMCs)_resting | 13.9 | 4.1 |
| 93113_Mononuclear Cells (PBMCs)_PWM | 32.1 | 13.0 |
| 93114_Mononuclear Cells (PBMCs)_PHA-L | 20.4 | 8.3 |
| 93249_Ramos (B cell)_none | 15.5 | 5.3 |
| 93250_Ramos (B cell)_ionomycin | 35.4 | 12.8 |
| 93349_B lymphocytes_PWM | 38.7 | 9.2 |
| 93350_B lymphoytes_CD40L and IL-4 | 30.8 | 8.5 |
| 92665_EOL-1 (Eosinophil)_dbcAMP differentiated | 8.1 | 2.3 |
| 93248_EOL-1 (Eosinophil)_dbcAMP/PMAionomycin | 32.5 | 8.4 |
| 93356_Dendritic Cells_none | 31.4 | 10.4 |
| 93355_Dendritic Cells_LPS 100 ng/ml | 49.0 | 14.0 |
| 93775_Dendritic Cells_anti-CD40 | 33.4 | 10.5 |
| 93774_Monocytes_resting | 18.3 | 5.9 |
| 93776_Monocytes_LPS 50 ng/ml | 44.4 | 18.9 |
| 93581_Macrophages_resting | 37.4 | 14.0 |
| 93582_Macrophages_LPS 100 ng/ml | 57.0 | 16.1 |
| 93098_HUVEC (Endothelial)_none | 34.9 | 9.1 |
| 93099_HUVEC (Endothelial)_starved | 54.7 | 15.6 |
| 93100_HUVEC (Endothelial)_IL-lb | 10.2 | 2.8 |
| 93779_HUVEC (Endothelial)_IFN gamma | 38.4 | 10.2 |
| 93102_HUVEC (Endothelial)_TNF alpha + IFN gamma | 42.0 | 13.9 |
| 93101_HUVEC (Endothelial)_TNF alpha + IL4 | 38.4 | 11.5 |
| 93781_HUVEC (Endothelial)_IL-11 | 16.8 | 6.1 |
| 93583_Lung Microvascular Endothelial Cells_none | 38.4 | 13.5 |
| 93584_Lung Microvascular Endothelial Cells_TNFa (4 ng/ml) | 32.8 | 100.0 |
| and IL1b (1 ng/ml) | | |
| 92662_Microvascular Dermal endothelium_none | 38.7 | 16.0 |
| 92663_Microsvasular Dermal endothelium_TNFa (4 ng/ml) and | 28.3 | 8.3 |
| IL1b (1 ng/ml) | | |
| 93773_Bronchial epithelium_TNFa (4 ng/ml) and ILIb (1 | 9.7 | 27.8 |
| ng/ml)** | | |
| 93347_Small Airway Epithelium_none | 29.1 | 10.2 |
| 93348_Small Airway Epithelium_TNFa (4 ng/ml) and IL1b (1 | 100.0 | 34.1 |
| ng/ml) | | |
| 92668_Coronery Artery SMC_resting | 62.4 | 13.2 |
| 92669_Coronery Artery SMC_TNFa (4 ng/ml) and IL1b (1 | 64.2 | 12.1 |
| ng/ml) | | |
| 93107_astrocytes_resting | 43.8 | 12.6 |
| 93108_astrocytes_TNFa (4 ng/ml) and IL1b (1 ng/ml) | 44.8 | 10.6 |
| 92666_KU-812 (Basophil)_resting | 4.8 | 1.4 |
| 92667_KU-812 (Basophil)_PMA/ionoycin | 35.8 | 11.2 |
| 93579_CCD1106 (Keratinocytes)_none | 24.7 | 11.0 |
| 93580_CCD1106 (Keratinocytes)_TNFa and IFNg** | 6.9 | 13.9 |
| 93791_Liver Cirrhosis | 5.1 | 3.0 |
| 93792_Lupus Kidney | 7.1 | 2.3 |
| 93577_NCI-H292 | 40.3 | 14.3 |
| 93358_NCI-H292_IL-4 | 62.4 | 17.8 |
| 93360_NCI-H292_IL-9 | 55.9 | 15.7 |
| 93359_NCI-H292_IL-13 | 51.0 | 13.7 |
| 93357_NCI-H292_IFN gamma | 45.1 | 14.5 |
| 93777_HPAEC_- | 35.4 | 9.4 |
| 93778_HPAEC_IL-1 beta/TNA alpha | 45.4 | 17.3 |
| 93254_Normal Human Lung Fibroblast_none | 44.1 | 14.1 |
| 93253_Normal Human Lung Fibroblast_TNFa (4 ng/ml) and IL- | 49.0 | 16.8 |
| 1b (1 ng/ml) | | |
| 93257_Normal Human Lung Fibroblast_IL-4 | 73.7 | 22.4 |
| 93256_Normal Human Lung Fibroblast_IL-9 | 55.1 | 18.8 |
| 93255_Normal Human Lung Fibroblast_IL-13 | 45.1 | 14.3 |
| 93258_Normal Human Lung Fibroblast_IFN gamma | 87.1 | 30.4 |
| 93106_Dermal Fibroblasts CCD1070_resting | 59.0 | 17.6 |
| 93361_Dermal Fibroblasts CCD1070_TNF alpha 4 ng/ml | 81.8 | 19.2 |
| 93105_Dermal Fibroblasts CCD1070_IL-1 beta 1 ng/ml | 41.5 | 10.5 |
| 93772_dermal fibroblast_IFN gamma | 48.6 | 13.9 |
| 93771_dermal fibroblast_IL-4 | 70.7 | 20.3 |
| 93259_IBD Colitis 1** | 1.4 | 4.5 |
| 93260_IBD Colitis 2 | 1.3 | 0.3 |
| 93261_IBD Crohns | 2.6 | 0.7 |
| 735010_Colon_normal | 51.0 | 13.6 |
| 735019_Lung_none | 41.2 | 10.9 |
| 64028-1_Thymus_none | 41.8 | 11.3 |
| 64030-1_Kidney_none | 26.4 | 7.8 |

Panel 1 Summary: The NOV2 gene appears to be expressed highly in most of the tissues on this panel irrespective of whether the sample was from normal tissue or a cancer cell line. Expression was highest in the trachea. These results suggest that the function of the NOV2 gene may be important for a variety of tissues.

Panel 1.2 Summary: Three separate RTQ PCR experiments have been run to look at expression of the NOV2 gene; two are in good agreement and are presumed to reflect the true expression pattern of this gene. The NOV2 gene appears to be expressed highly in most of the tissues on this panel irrespective of whether the sample was from normal tissue or a cancer cell line. Expression was highest in an ovarian carcinoma cell line; this may suggest that the NOV2 gene plays a role in a subset of ovarian cancers. The results from this panel suggest that the function of the NOV2 gene may be important for a variety of tissues.

Panel 1.3D Summary: Two separate RTQ PCR experiments using different probe and primer sets have been run to look at expression of the NOV2 gene; the results are in reasonable agreement with some minor differences. In general, the NOV2 gene appears to be expressed highly in most of the tissues on this panel irrespective of whether the sample was from normal tissue or a cancer cell line. Expression was highest in fetal skeleton. Expression of the NOV2 gene within the brain was highest in the hippocampus and amygdala. The LDL-Receptor Related Protein (LRP) has been implicated in Alzheimer's disease through several independent lines of research. LRP is a receptor for apoE, and one of the three common apoE alleles (apoE epsilon4) has been shown to increase the risk of late-onset Alzheimer's disease. Because apoE binds amyloid beta (the protein responsible for the primary pathology of Alzheimer's disease; the senile plaque), it has been suggested that A-Beta clearance occurs through apoE-mediated uptake via the LRP. Furthermore, mutations in the LRP may increase the risk of AD. This protein may therefore be of use in the treatment of Alzheimer's by upregulating A-beta clearance. The LRP also is involved in cholesterol transport within the brain, and has been implicated in compensatory synaptogenesis (specifically in the transport of hydrophobic membrane components). Thus, in any neurodegenerative disease/ brain trauma where neuronal death occurs, this protein may be of use in the response to injury for selectively increasing compensatory synaptogenesis.

Panel 2D Summary: Two separate RTQ PCR experiments using different probe and primer sets have been run to look at expression of the NOV2 gene on panel 2D. In both cases, expression of the NOV2 gene is high in most of the tissues regardless of whether the sample was from normal tissue or a tumor. However, there were also some discrepancies between the results from the two experiments for unclear reasons; one possibility is that splice variants of this protein exist thiat are differentially expressed. In one experiment, NOV2 gene expression was highest in a normal kidney sample, while in the other it was highest in normal prostate.

Panel 3D Summary: Ubiquitous high expression of the NOV2 gene was detected in all of the cancer cell line samples on this panel, with highest expression in pancreatic ductal adenocarcinoma. These results are consistent with what was seen on the other panels and further provide support to the notion that this gene plays a role in the function of all major cell types.

Panel 4D Summary: For Ag995, there is high expression of this transcript regardless of treatment in most tissues. The exception is in colon from patients with inflammatory bowel disease. Normal colon expresses high levels; whereas, inflamed bowel expresses relatively low levels of the transcript. Agonistic protein therapeutics to this antigen could therefore reduce or block the inflammatory process during IBD. The findings with Ag2749 are consistent with the findings with Ag995 with the exception of transcript expression by lung microvascular endothelial cells treated with TNFalpha and IL-1 beta. The high transcript expression by these endothelial cells appears to be a PCR artifact based on the amplification plot.

NOV4A

Expression of NOV4a was assessed using the primer-probe sets Ag2650 and Ag1072, described in Tables T and U. Results of the RTQ-PCR runs are shown in Tables V, W, X and Y.

TABLE T
Probe Name: Ag2650

| | | | | Start |
|---|---|---|---|---|
| Primers | Sequences | TM | Length | Position |
| Forward | 5′-GGTGCAGCTGAGATT | 58 | 20 | 168 |
| | CAAGT-3′ (SEQ ID | | | |
| | NO: 119) | | | |
| Probe | FAM-5′-CCGATCTCCAGGAG | 68.8 | 26 | 202 |
| | CTATGTCAGACA-3′-TAMRA | | | |
| | (SEQ ID NO: 120) | | | |
| Reverse | 5′-TGACATCAGTACCACCTT | 59.1 | 22 | 243 |
| | CACA-3′ SEQ ID | | | |
| | NO: 121) | | | |

TABLE U
Probe Name Ag1072

| | | | | Start |
|---|---|---|---|---|
| Primers | Sequences | TM | Length | Position |
| Forward | 5′-TATGCTTGAACCCACTG | 59.1 | 21 | 281 |
| | ATGA-3′ (SEQ ID | | | |
| | NO: 122) | | | |
| Probe | TET-5′-AGAGCCTAAAGAAGA | 65.6 | 26 | 302 |
| | GAAACCACCCA-3′-TAMRA | | | |
| | (SEQ ID NO: 123) | | | |
| Reverse | 5′-TTCTGATCAGGTGTAGGA | 58.1 | 22 | 337 |
| | TTCC-3′ (SEQ ID | | | |
| | NO: 124) | | | |

TABLE V
Panel 1.2

| | Relative | Relative | Relative |
|---|---|---|---|
| | Expression (%) | Expression (%) | Expression (%) |
| Tissue Name | 1.2tm1180t_ag1072 | 1.2tm1292t_ag1072 | 1.2tm1314t_ag1072 |
| Endothelial cells | 8.0 | 0.0 | 0.0 |
| Endothelial cells (treated) | 0.0 | 0.0 | 0.0 |
| Pancreas | 4.2 | 0.0 | 0.0 |
| Pancreatic ca. CAPAN 2 | 1.1 | 0.0 | 0.0 |
| Adrenal Gland (new lot*) | 46.0 | 0.0 | 0.0 |
| Thyroid | 0.6 | 0.0 | 0.0 |
| Salivary gland | 13.8 | 0.0 | 0.0 |
| Pituitary gland | 7.9 | 0.0 | 0.0 |
| Brain (fetal) | 4.9 | 0.0 | 0.0 |
| Brain (whole) | 8.0 | 0.0 | 0.2 |
| Brain (amygdala) | 17.2 | 0.0 | 0.0 |
| Brain (cerebellum) | 23.0 | 0.0 | 0.0 |
| Brain (hippocampus) | 17.2 | 0.0 | 0.0 |
| Brain (thalamus) | 1.9 | 0.0 | 0.0 |
| Cerebral Cortex | 8.7 | 0.0 | 0.0 |
| Spinal cord | 4.1 | 0.0 | 0.0 |
| CNS ca. (glio/astro) U87-MG | 8.8 | 0.0 | 0.0 |
| CNS ca. (glio/astro) U-118-MG | 0.3 | 0.0 | 0.0 |
| CNS ca. (astro) SW1783 | 0.7 | 0.0 | 0.0 |
| CNS ca.* (neuro; met) SK-N-AS | 12.9 | 0.0 | 0.0 |
| CNS ca. (astro) SF-539 | 6.1 | 0.0 | 0.0 |
| CNS ca. (astro) SNB-75 | 0.1 | 0.0 | 0.0 |
| CNS ca. (glio) SNB-19 | 13.6 | 0.0 | 0.0 |
| CNS ca. (glio) U251 | 1.0 | 0.0 | 0.0 |
| CNS ca. (glio) SF-295 | 1.6 | 0.0 | 0.0 |
| Heart | 92.7 | 0.0 | 0.0 |
| Skeletal Muscle (new lot*) | 56.6 | 0.0 | 0.0 |
| Bone marrow | 0.4 | 0.0 | 0.0 |
| Thymus | 0.2 | 0.0 | 0.0 |
| Spleen | 0.0 | 0.0 | 0.0 |
| Lymph node | 1.2 | 0.0 | 0.0 |
| Colorectal | 0.0 | 0.0 | 0.0 |
| Stomach | 8.5 | 0.0 | 0.0 |
| Small intestine | 19.9 | 0.0 | 0.0 |
| Colon ca. SW480 | 0.0 | 0.0 | 0.0 |
| Colon ca.* (SW480 met) SW620 | 9.2 | 0.0 | 0.0 |
| Colon ca. HT29 | 0.2 | 0.0 | 0.0 |
| Colon ca. HCT-116 | 9.2 | 0.0 | 0.0 |
| Colon ca. CaCo-2 | 1.6 | 0.8 | 2.0 |
| 83219 CC Well to Mod Diff (ODO3866) | 0.1 | 0.0 | 0.0 |
| Colon ca. HCC-2998 | 7.0 | 0.0 | 0.0 |
| Gastric ca.* (liver met) NCI-N87 | 8.2 | 0.0 | 0.0 |
| Bladder | 4.8 | 0.0 | 0.0 |
| Trachea | 5.1 | 0.0 | 0.0 |
| Kidney | 33.7 | 0.0 | 0.0 |
| Kidney (fetal) | 100.0 | 0.0 | 0.0 |
| Renal ca. 786-0 | 3.7 | 0.0 | 0.0 |
| Renal ca. A498 | 1.4 | 0.0 | 0.0 |
| Renal ca. RXF 393 | 0.2 | 0.0 | 0.0 |
| Renal ca. ACHN | 11.0 | 0.0 | 0.0 |
| Renal ca. UO-31 | 1.7 | 0.0 | 0.0 |
| Renal ca. TK-10 | 4.6 | 0.0 | 0.0 |
| Liver | 10.3 | 0.0 | 0.0 |
| Liver (fetal) | 5.1 | 0.0 | 0.0 |
| Liver ca. (hepatoblast) HepG2 | 5.5 | 0.0 | 0.0 |
| Lung | 9.5 | 0.0 | 0.4 |
| Lung (fetal) | 5.0 | 0.0 | 0.5 |
| Lung ca. (small cell) LX-1 | 1.2 | 0.0 | 0.0 |
| Lung ca. (small cell) NCI-H69 | 21.0 | 0.0 | 0.0 |
| Lung ca. (s. cell var.) SHP-77 | 4.7 | 0.0 | 0.0 |
| Lung ca. (large cell) NCI-H460 | 6.8 | 0.0 | 0.0 |
| Lung ca. (non-sm. cell) A549 | 7.8 | 0.0 | 0.0 |
| Lung ca. (non-s. cell) NCI-H23 | 7.1 | 0.0 | 0.0 |
| Lung ca (non-s. cell) HOP-62 | 0.8 | 0.0 | 0.0 |
| Lung ca. (non-s. cl) NCI-H522 | 18.4 | 0.0 | 0.0 |
| Lung ca. (squam.) SW 900 | 10.3 | 0.0 | 0.0 |
| Lung ca. (squam.) NCI-H596 | 6.7 | 0.0 | 0.0 |
| Mammary gland | 12.2 | 0.0 | 0.0 |
| Breast ca.* (pl. effusion) MCF-7 | 14.1 | 0.0 | 0.0 |
| Breast ca.* (pl.ef) M1DA-MB-23 | 6.0 | 0.0 | 0.0 |
| Breast ca.* (pl. effusion) T47D | 2.6 | 0.0 | 1.0 |
| Breast ca. BT-549 | 6.9 | 0.0 | 0.0 |
| Breast ca. MDA-N | 5.1 | 0.0 | 0.0 |
| Ovary | 0.1 | 0.0 | 0.0 |
| Ovarian ca. OVCAR-3 | 5.3 | 0.0 | 0.6 |
| Ovarian ca. OVCAR-4 | 15.8 | 0.0 | 0.0 |
| Ovarian ca. OVCAR-5 | 3.4 | 0.0 | 0.0 |
| Ovarian ca. OVCAR-8 | 5.6 | 0.0 | 0.0 |
| Ovarian ca. IGROV-1 | 4.9 | 0.0 | 0.0 |
| Ovarian ca.* (ascites) SK-OV-3 | 9.7 | 0.0 | 0.0 |
| Uterus | 7.8 | 0.0 | 0.0 |
| Placenta | 0.0 | 100.0 | 100.0 |
| Prostate | 20.6 | 0.0 | 0.0 |
| Prostate ca.* (bone met)PC-3 | 42.3 | 0.0 | 0.0 |
| Testis | 9.9 | 0.0 | 0.2 |
| Melanoma Hs688(A).T | 1.0 | 0.0 | 0.0 |
| Melanoma* (met) Hs688(B).T | 3.5 | 0.0 | 0.0 |
| Melanoma UACC-62 | 3.5 | 0.0 | 0.0 |
| Melanoma M14 | 6.5 | 0.0 | 0.0 |
| Melanoma LOX IMVI | 6.9 | 0.0 | 0.0 |
| Melanoma* (met) SK-MEL-5 | 4.8 | 0.0 | 0.0 |
| Adipose | 2.2 | 0.0 | 0.0 |

TABLE W
Panel 1.3D

| | Relative | | Relative |
|---|---|---|---|
| | Expression (%) | | Expression (%) |
| Tissue Name | 1.3Dtm3426f_ag2650 | Tissue Name | 1.3Dtm3426f_ag2650 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.1 |
| Brain (amygdala) | 0.0 | Liver ca. (hepatoblast) HepG2 | 0.0 |
| Brain (cerebellum) | 0.0 | Lung | 1.0 |
| Brain (hippocampus) | 0.0 | Lung (fetal) | 0.6 |
| Brain (substantia nigra) | 0.0 | Lung ca. (small cell) LX-1 | 0.2 |
| Brain (thalamus) | 0.0 | Lung ca. (small cell) NCI-H69 | 0.0 |
| Cerebral Cortex | 0.0 | Lung ca. (s. cell var.) SHP-77 | 0.0 |
| Spinal cord | 0.0 | Lung ca. (large cell)NCI-H460 | 0.0 |
| CNS ca. (glio/astro) U87-MG | 0.0 | Lung ca. (non-sm. cell) A549 | 0.0 |
| CNS ca. (glio/astro) U-118-MG | 0.0 | Lung ca. (non-s. cell) NCI-H23 | 0.0 |
| CNS ca (astro) SW1783 | 0.0 | Lung ca (non-s. cell) HOP-62 | 0.0 |
| CNS ca.* (neuro; met) SK-N-AS | 0.0 | Lung ca. (non-s. cl) NCI-H522 | 0.0 |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 0.0 |
| CNS ca. (glio) SNB-19 | 0.0 | Mammary gland | 0.0 |
| CMS ca. (glio) U251 | 0.0 | Breast ca.* (pl. effusion) MCF-7 | 0.0 |
| CNS ca. (glio) SF-295 | 0.0 | Breast ca.* (pl.ef) MDA-MB-231 | 0.0 |
| Heart (fetal) | 0.0 | Breast ca.* (pl. effusion) T47D | 2.2 |
| Heart | 0.0 | Breast ca. BT-549 | 0.0 |
| Fetal Skeletal | 0.1 | Breast ca. MDA-N | 0.0 |
| Skeletal muscle | 0.0 | Ovary | 0.0 |
| Bone marrow | 0.0 | Ovarian ca. OVCAR-3 | 0.7 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca.* (ascites) SK-OV-3 | 0.0 |
| Small intestine | 0.0 | Uterus | 0.0 |
| Colon ca. SW480 | 0.2 | Placenta | 100.0 |
| Colon ca.* (SW480 met)SW620 | 0.0 | Prostate | 0.0 |
| Colon ca. HT29 | 0.0 | Prostate ca.* (bone met)PC-3 | 0.0 |
| Colon ca. HCT-116 | 0.0 | Testis | 0.1 |
| Colon ca. CaCo-2 | 2.9 | Melanoma Hs688(A).T | 0.0 |
| 83219 CC Well to Mod Diff (ODO3866) | 0.0 | Melanoma* (met) Hs688(B).T | 0.0 |
| Colon ca. HCC-2998 | 0.0 | Melanoma UACC-62 | 0.0 |
| Gastric ca.* (liver met) NCI-N87 | 0.0 | Melanoma M14 | 0.0 |
| Bladder | 0.0 | Melanoma LOX IMVI | 0.0 |
| Trachea | 0.0 | Melanoma* (met) SK-MEL-5 | 0.0 |
| Kidney | 0.0 | Adipose | 0.0 |

TABLE X
Panel 2D

| | Relative |
|---|---|
| | Expression (%) |
| Tissue Name | 2Dtm3427f_ag2650 |
| Normal Colon GENPAK | 0.5 |
| 061003 | |
| 83219 CC Well to Mod Diff | 0.0 |
| (ODO3866) | |
| 83220 CC NAT (ODO3866) | 0.0 |
| 83221 CC Gr.2 rectosigmoid | 0.0 |
| (ODO3868) | |
| 83222 CC NAT (ODO3868) | 0.0 |
| 83235 CC Mod Diff | 2.6 |
| (ODO3920) | |
| 83236 CC NAT (ODO3920) | 0.0 |
| 83237 CC Gr.2 ascend colon | 0.0 |
| (ODO3921) | |
| 83238 CC NAT (ODO3921) | 0.0 |
| 83241 CC from Partial | 0.2 |
| Hepatectomy (ODO4309) | |
| 83242 Liver NAT (ODO4309) | 0.0 |
| 87472 Colon mets to lung | 6.4 |
| (OD04451-01) | |
| 87473 Lung NAT (OD04451- | 4.6 |
| 02) | |
| Normal Prostate Clontech A + | 0.0 |
| 6546-1 | |
| 84140 Prostate Cancer | 0.0 |
| (OD04410) | |
| 84141 Prostate NAT | 0.0 |
| (OD04410) | |
| 87073 Prostate Cancer | 0.0 |
| (OD04720-01) | |
| 87074 Prostate NAT | 0.0 |
| (OD04720-02) | |
| Normal Lung GENPAK 061010 | 16.2 |
| 83239 Lung Met to Muscle | 0.0 |
| (ODO4286) | |
| 83240 Muscle NAT | 0.0 |
| (ODO4286) | |
| 84136 Lung Malignant Cancer | 5.8 |
| (OD03126) | |
| 84137 Lung NAT (OD03126) | 100.0 |
| 84871 Lung Cancer (OD04404) | 1.4 |
| 84872 Lung NAT (OD04404) | 11.0 |
| 84875 Lung Cancer (OD04565) | 0.0 |
| 84876 Lung NAT (OD04565) | 4.0 |
| 85950 Lung Cancer (OD04237- | 1.5 |
| 01) | |
| 85970 Lung NAT (OD04237- | 18.3 |
| 02) | |
| 83255 Ocular Mel Met to Liver | 52.8 |
| (ODO4310) | |
| 83256 Liver NAT (ODO4310) | 0.0 |
| 84139 Melanoma Mets to Lung | 0.0 |
| (OD04321) | |
| 84138 Lung NAT (OD04321) | 9.9 |
| Normal Kidney GENPAK | 0.6 |
| 061008 | |
| 83786 Kidney Ca, Nuclear | 0.0 |
| grade 2 (OD04338) | |
| 83787 Kidney NAT (OD04338) | 0.3 |
| 83788 Kidney Ca Nuclear grade | 0.0 |
| ½ (OD04339) | |
| 83789 Kidney NAT (OD04339) | 0.5 |
| 83790 Kidney Ca, Clear cell | 0.0 |
| type (OD04340) | |
| 83791 Kidney NAT (OD04340) | 1.1 |
| 83792 Kidney Ca, Nuclear | 0.0 |
| grade 3 (OD04348) | |
| 83793 Kidney NAT (OD04348) | 0.2 |
| 87474 Kidney Cancer | 0.2 |
| (OD04622-01) | |
| 87475 Kidney NAT (OD04622- | 0.0 |
| 03) | |
| 85973 Kidney Cancer | 0.0 |
| (ODO4450-01) | |
| 85974 Kidney NAT (OD04450- | 0.7 |
| 03) | |
| Kidney Cancer Clontech | 0.0 |
| 8120607 | |
| Kidney NAT Clontech 8120608 | 0.0 |
| Kidney Cancer Clontech | 0.0 |
| 8120613 | |
| Kidney NAT Clontech 8120614 | 0.3 |
| Kidney Cancer Clontech | 1.1 |
| 9010320 | |
| Kidney NAT Clontech 9010321 | 0.6 |
| Normal Uterus GENPAK | 0.0 |
| 061018 | |
| Uterus Cancer GENPAK | 0.0 |
| 064011 | |
| Normal Thyroid Clontech A + | 0.0 |
| 6570-1 | |
| Thyroid Cancer GENPAK | 0.2 |
| 064010 | |
| Thyroid Cancer INVITROGEN | 0.0 |
| A302152 | |
| Thyroid NAT INVITROGEN | 0.0 |
| A302153 | |
| Normal Breast GENPAK | 0.0 |
| 061019 | |
| 84877 Breast Cancer | 0.0 |
| (OD04566) | |
| 85975 Breast Cancer | 0.0 |
| (OD04590-01) | |
| 85976 Breast Cancer Mets | 0.0 |
| (OD04590-03) | |
| 87070 Breast Cancer Metastasis | 0.0 |
| (OD04655-05) | |
| GENPAK Breast Cancer | 0.0 |
| 064006 | |
| Breast Cancer Res. Gen. 1024 | 0.2 |
| Breast Cancer Clontech | 0.0 |
| 9100266 | |
| Breast NAT Clontech 9100265 | 0.0 |
| Breast Cancer INVITROGEN | 0.0 |
| A209073 | |
| Breast NAT INVITROGEN | 0.0 |
| A2090734 | |
| Normal Liver GENPAK | 0.0 |
| 061009 | |
| Liver Cancer GENPAK 064003 | 0.0 |
| Liver Cancer Research Genetics | 0.0 |
| RNA 1025 | |
| Liver Cancer Research Genetics | 0.0 |
| RNA 1026 | |
| Paired Liver Cancer Tissue | 0.0 |
| Research Genetics RNA 6004- | |
| T | |
| Paired Liver Tissue Research | 0.0 |
| Genetics RNA 6004-N | |
| Paired Liver Cancer Tissue | 0.0 |
| Research Genetics RNA 6005- | |
| T | |
| Paired Liver Tissue Research | 0.0 |
| Genetics RNA 6005-N | |
| Normal Bladder GENPAK | 0.2 |
| 061001 | |
| Bladder Cancer Research | 0.3 |
| Genetics RNA 1023 | |
| Bladder Cancer INVITROGEN | 0.3 |
| A302173 | |
| 87071 Bladder Cancer | 71.7 |
| (OD04718-01) | |
| 87072 Bladder Normal | 0.2 |
| Adjacent (OD04718-03) | |
| Normal Ovary Res. Gen. | 0.0 |
| Ovarian Cancer GENPAK | 0.0 |
| 064008 | 0.0 |
| 87492 Ovary Cancer | 0.4 |
| (OD04768-07) | |
| 87493 Ovary NAT (OD04768- | 0.0 |
| 08) | |
| Normal Stomach GENPAK | 0.0 |
| 061017 | |
| Gastric Cancer Clontech | 0.0 |
| 9060358 | |
| NAT Stomach Clontech | 0.0 |
| 9060359 | |
| Gastric Cancer Clontech | 0.0 |
| 9060395 | |
| NAT Stomach Clontech | 0.0 |
| 9060394 | |
| Gastric Cancer Clontech | 0.0 |
| 9060397 | |
| NAT Stomach Clontech | 0.0 |
| 9060396 | |
| Gastric Cancer GENPAK | 0.0 |
| 064005 | |

TABLE Y
Panel 4D

| | Relative | Relative |
|---|---|---|
| | Expression (%) | Expression (%) |
| Tissue Name | 4dtm2466t_ag1072 | 4Dtm3428f_ag2650 |
| 93768_Secondary Th1_anti-CD28/anti-CD3 | 0.0 | 0.0 |
| 93769_Secondary Th2_anti-CD28/anti-CD3 | 0.0 | 0.0 |
| 93770_Secondary Tr1_anti-CD28/anti-CD3 | 0.0 | 0.0 |
| 93573_Secondary Th1_resting day 4-6 in IL-2 | 0.0 | 0.0 |
| 93572_Secondary Th2_resting day 4-6 in IL-2 | 0.0 | 0.0 |
| 93571_Secondary Tr1_resting day 4-6 in IL-2 | 0.0 | 0.0 |
| 93568_primary Th1_anti-CD28/anti-CD3 | 0.0 | 0.0 |
| 93569_primary Th2_anti-CD28/anti-CD3 | 0.0 | 0.0 |
| 93570_primary Tr1_anti-CD28/anti-CD3 | 0.0 | 0.0 |
| 93565_primary Th1_resting dy 4-6 in IL-2 | 0.0 | 0.0 |
| 93566_primary Th2_resting dy 4-6 in IL-2 | 0.0 | 0.0 |
| 93567_primary Tr1_resting dy 4-6 in IL-2 | 0.0 | 0.0 |
| 93351_CD45RA CD4 lymphocyte_anti-CD28/anti-CD3 | 5.0 | 0.0 |
| 93352_CD45RO CD4 lymphocyte_anti-CD28/anti-CD3 | 0.0 | 0.0 |
| 93251_CD8 Lymphocytes_anti-CD28/anti-CD3 | 0.0 | 0.0 |
| 93353_chronic CD8 Lymphocytes 2ry_resting dy 4-6 in IL-2 | 0.0 | 0.0 |
| 93574_chronic CD8 Lymphocytes 2ry_activated CD3/CD28 | 0.0 | 0.0 |
| 93354_CD4_none | 0.0 | 0.0 |
| 93252_Secondary Th1/Th2/Tr1_anti-CD95 CH11 | 0.0 | 0.0 |
| 93103_LAK cells_resting | 0.0 | 0.0 |
| 93788_LAK cells_IL-2 | 0.0 | 0.0 |
| 93787_LAK cells_IL-2 + IL-12 | 0.0 | 0.0 |
| 93789_LAK cells_IL-2 + IFN gamma | 8.9 | 0.0 |
| 93790_LAK cells_IL-2 + IL-18 | 0.0 | 0.0 |
| 93104_LAK cells_PMA/ionomycin and IL-18 | 0.0 | 0.0 |
| 93578_NK Cells IL-2_resting | 0.0 | 0.0 |
| 93109_Mixed Lymphocyte Reaction_Two Way MLR | 0.0 | 0.0 |
| 93110_Mixed Lymphocyte Reaction_Two Way MLR | 0.0 | 0.0 |
| 93111_Mixed Lymphocyte Reaction_Two Way MLR | 0.0 | 0.0 |
| 93112_Mononuclear Cells (PBMCs)_resting | 0.0 | 0.0 |
| 93113_Mononuclear Cells (PBMCs)_PWM | 0.0 | 0.0 |
| 93114_Mononuclear Cells (PBMCs)_PHA-L | 0.0 | 0.0 |
| 93249_Ramos (B cell)_none | 0.0 | 0.0 |
| 93250_Ramos (B cell)_ionomycin | 0.0 | 0.0 |
| 93349_B lymphocytes_PWM | 0.0 | 0.0 |
| 93350_B lymphoytes_CD40L and IL-4 | 0.0 | 0.0 |
| 92665_EOL-1 (Eosinophil)_dbcAMP differentiated | 0.0 | 0.0 |
| 93248_EOL-1 (Eosinophil)_dbcAMP/PMAionomycin | 0.0 | 0.0 |
| 93356_Dendritic Cells_none | 0.0 | 0.0 |
| 93355_Dendritic Cells_LPS 100 ng/ml | 0.0 | 0.0 |
| 93775_Dendritic Cells_anti-CD40 | 0.0 | 0.0 |
| 93774_Monocytes_resting | 0.0 | 0.0 |
| 93776_Monocytes_LPS 50 ng/ml | 0.0 | 0.0 |
| 93581_Macrophages_resting | 0.0 | 0.0 |
| 93582_Macrophages_LPS 100 ng/ml | 0.0 | 0.0 |
| 93098_HUVEC (Endothelial)_none | 0.0 | 0.0 |
| 93099_HUVEC (Endothelial)_starved | 0.0 | 0.0 |
| 93100_HUVEC (Endothelial)_IL-1b | 0.0 | 0.0 |
| 93779_HUVEC (Endothelial)_IFN gamma | 0.0 | 0.0 |
| 93102_HUVEC (Endothelial)_TNF alpha + IFN gamma | 0.0 | 0.0 |
| 93101_HUVEC (Endothelial)_TNF alpha + IL4 | 0.0 | 0.0 |
| 93781_HUVEC (Endothelial)_IL-11 | 0.0 | 0.0 |
| 93583_Lung Microvascular Endothelial Cells_none | 0.0 | 0.0 |
| 93584_Lung Microvascular Endothelial Cells_TNFa (4 ng/ml) | 0.0 | 0.0 |
| and IL1b (1 ng/ml) | | |
| 92662_Microvascular Dermal endothelium_none | 0.0 | 0.0 |
| 92663_Microsvasular Dermal endothelium_TNFa_(4 ng/ml) and | 0.0 | 0.0 |
| IL1b (1 ng/ml) | | |
| 93773_Bronchial epithelium_TNFa (4 nglml) and IL1b (1 | 0.0 | 0.0 |
| ng/ml)** | | |
| 93347_Small Airway Epithelium_none | 0.0 | 0.0 |
| 93348_Small Airway Epithelium_TNFa (4 ng/ml) and IL1b (1 | 0.0 | 0.0 |
| ng/ml) | | |
| 92668_Coronery Artery SMC_resting | 0.0 | 0.0 |
| 92669_Coronery Artery SMC_TNFa (4 ng/ml) and IL1b (1 | 0.0 | 0.0 |
| ng/ml) | | |
| 93107_astrocytes_resting | 0.0 | 0.0 |
| 93108_astrocytes_TNFa (4 ng/ml) and IL1b (1 ng/ml) | 0.0 | 0.0 |
| 92666_KU-812 (Basophil)_resting | 0.0 | 3.1 |
| 92667_KU-812 (Basophil)_PMA/ionoycin | 0.0 | 5.8 |
| 93579_CCD1106 (Keratinocytes)_none | 0.0 | 0.0 |
| 93580_CCD1106 (Keratinocytes)_TNFa and IFNg** | 0.0 | 0.0 |
| 93791_Liver Cirrhosis | 2.8 | 7.1 |
| 93792_Lupus Kidney | 0.0 | 0.0 |
| 93577_NCI-H292 | 37.4 | 76.3 |
| 93358_NCI-H292_IL-4 | 69.3 | 100.0 |
| 93360_NCI-H292_IL-9 | 100.0 | 80.7 |
| 93359_NCI-H292_IL-13 | 48.0 | 69.3 |
| 93357_NCI-H292_IFN gamma | 45.7 | 64.6 |
| 93777_HPAEC_- | 0.0 | 0.0 |
| 93778_HPAEC_IL-1 beta/TNA alpha | 0.0 | 0.0 |
| 93254_Normal Human Lung Fibroblast_none | 0.0 | 0.0 |
| 93253_Normal Human Lung Fibroblast_TNFa (4 ng/ml) and IL- | 0.0 | 0.0 |
| lb (1 ng/ml) | | |
| 93257_Normal Human Lung Fibroblast_IL-4 | 0.0 | 0.0 |
| 93256_Normal Human Lung Fibroblast_IL-9 | 0.0 | 0.0 |
| 93255_Normal Human Lung Fibroblast_IL-13 | 0.0 | 0.0 |
| 93258_Normal Human Lung Fibroblast_IFN gamma | 0.0 | 0.0 |
| 93106_Dermal Fibroblasts CCD1070_resting | 0.0 | 0.0 |
| 93361_Dermal Fibroblasts CCD1070_TNF alpha 4 ng/ml | 0.0 | 0.0 |
| 93105_Dermal Fibroblasts CCD1070_IL-1 beta 1 ng/ml | 0.0 | 0.0 |
| 93772_dermal fibroblast_IFN gamma | 0.0 | 0.0 |
| 93771_dermal fibroblast_IL-4 | 0.0 | 0.0 |
| 93259_IBD Colitis 1** | 0.0 | 0.0 |
| 93260_IBD Colitis 2 | 0.0 | 0.0 |
| 93261_IBD Crohns | 0.0 | 0.0 |
| 735010_Colon_normal | 0.0 | 0.0 |
| 735019_Lung_none | 99.3 | 70.7 |
| 64028-1_Thymus_none | 0.0 | 2.8 |
| 64030-1_Kidney_none | 0.0 | 0.0 |

Panel 1.2 Summary: Three separate RTQ PCR experiments have been run to look at expression of the NOV4a gene; two are in good agreement and are presumed to reflect the true expression pattern of this gene. Expression of this gene across the tissues in panel 1.2 reveals that its expression is largely restricted to placental tissue. Thus, this gene is expressed almost exclusively in the placenta and can be used to identify/differentiate these tissues from others.

Panel 1.3D Summary: Expression of this gene across the tissues in panel 1.3D reveals that its expression is largely restricted to placenta, in agreement with what was observed for panel 1.2. Thus, this gene is expressed almost exclusively in the placenta and can be used to identify/differentiate these tissues from others.

Panel 2D Summary: Expression of the NOV4a gene in panel 2D demonstrates that its expression is restricted to normal adjacent lung tissue when compared to lung cancer tissue. This is apparent in 4 of 4 paired tissue samples from panel 2D. In summary, taken together, the data suggest that the expression of this gene is associated with placental tissue and lung tissue. Given that both tissues are involved with the exchange of nutrients and/or soluble gasses, this gene could potentially be involved in processes related to this function. Thus the use of this gene may be beneficial in disorders of such processes. In addition, since there appears to be a difference in the expression of this gene between lung cancers and normal adjacent tissues this gene may also have utility in the treatment of lung cancer.

Panel 4D Summary: Two separate RTQ PCR experiments using different probe and primer sets have been run to look at expression of the NOV4a gene and the results are in reasonable agreement. The expression of the transcript is limited to normal lung and NC1-H292 cells. The expression pattern of the transcript suggests that it might be useful as a diagnostic tool to identify goblet cells within the lung. Additionally, protein therapeutics designed with the protein encoded for by this transcript could be useful in reducing or blocking inflammation or mucus production due to asthma, emphysema, or allergies.

NOV5

Expression of NOV5 was assessed using the primer-probe set Ag1078, described in Table Z. Results of the RTQ-PCR runs are shown in Tables AA, BB, and CC.

TABLE Z
Probe Name: Ag1078

| | | | | Start |
|---|---|---|---|---|
| Primers | Sequences | TM | Length | Position |
| Forward | 5′-CCTGGACAGTGCATTT | 59.1 | 20 | 525 |
| | GATC-3′ (SEQ ID | | | |
| | NO: 125) | | | |
| Probe | TET-5′-TCCACACATACTCG | 69 | 26 | 566 |
| | CTCTCTGCCAAT-3′-TAMRA | | | |
| | (SEQ ID NO: 126) | | | |
| Reverse | 5′-CTGGTCCGAACCTCGA | 59 | 20 | 601 |
| | TATT-3′ (SEQ ID | | | |
| | NO: 127) | | | |

TABLE AA
Panel 1.2

| | Relative | Relative |
|---|---|---|
| | Expression (%) | Expression (%) |
| Tissue Name | 1.2tm1181t_ag1078 | 1.2tm1336t_ag1078 |
| Endothelial cells | 1.1 | 2.3 |
| Endothelial cells (treated) | 6.2 | 6.0 |
| Pancreas | 0.9 | 2.0 |
| Pancreatic ca. CAPAN 2 | 0.0 | 0.0 |
| Adrenal Gland (new lot*) | 26.8 | 18.3 |
| Thyroid | 1.6 | 4.9 |
| Salivary gland | 9.4 | 8.2 |
| Pituitary gland | 12.3 | 5.3 |
| Brain (fetal) | 6.0 | 5.1 |
| Brain (whole) | 3.3 | 6.1 |
| Brain (amygdala) | 4.4 | 2.7 |
| Brain (cerebellum) | 2.2 | 3.3 |
| Brain (hippocampus) | 4.0 | 3.2 |
| Brain (thalamus) | 1.4 | 1.9 |
| Cerebral Cortex | 12.4 | 6.7 |
| Spinal cord | 6.8 | 4.3 |
| CNS ca. (glio/astro) U87-MG | 1.4 | 0.7 |
| CNS ca. (glio/astro) U-118-MG | 25.5 | 20.9 |
| CNS ca. (astro) SW1783 | 1.4 | 0.6 |
| CNS ca.* (neuro; met) SK-N-AS | 38.7 | 60.3 |
| CNS ca. (astro) SF-539 | 4.6 | 4.6 |
| CNS ca. (astro) SNB-75 | 2.8 | 4.7 |
| CNS ca. (glio) SNB-19 | 1.7 | 2.1 |
| CNS ca. (glio) U251 | 1.3 | 2.3 |
| CNS ca. (glio) SF-295 | 55.1 | 76.3 |
| Heart | 24.8 | 12.9 |
| Skeletal Muscle (new lot*) | 3.3 | 6.0 |
| Bone marrow | 1.4 | 0.9 |
| Thymus | 1.6 | 0.8 |
| Spleen | 2.0 | 2.0 |
| Lymph node | 2.5 | 3.0 |
| Colorectal | 8.7 | 3.3 |
| Stomach | 17.8 | 15.9 |
| Small intestine | 21.5 | 20.3 |
| Colon ca. SW480 | 0.0 | 0.0 |
| Colon ca.* (SW480 met)SW620 | 0.0 | 0.0 |
| Colon ca. HT29 | 0.0 | 0.0 |
| Colon ca. HCT-116 | 0.0 | 0.0 |
| Colon ca. CaCo-2 | 0.4 | 0.5 |
| 83219 CC Well to Mod Diff (ODO3866) | 6.4 | 4.4 |
| Colon ca. HCC-2998 | 0.0 | 0.0 |
| Gastric ca.* (liver met) NCI-N87 | 0.0 | 0.0 |
| Bladder | 13.0 | 14.1 |
| Trachea | 6.3 | 2.3 |
| Kidney | 7.9 | 7.2 |
| Kidney (fetal) | 100.0 | 55.1 |
| Renal ca. 786-0 | 0.0 | 0.0 |
| Renal ca. A498 | 0.0 | 0.0 |
| Renal ca. RXF 393 | 0.0 | 0.0 |
| Renal ca. ACHN | 0.7 | 0.6 |
| Renal ca. UO-31 | 0.2 | 0.4 |
| Renal ca. TK-10 | 0.0 | 0.0 |
| Liver | 10.2 | 7.7 |
| Liver (fetal) | 7.0 | 4.1 |
| Liver ca. (hepatoblast) HepG2 | 0.0 | 0.0 |
| Lung | 7.5 | 3.5 |
| Lung (fetal) | 23.3 | 15.6 |
| Lung ca. (small cell) LX-1 | 0.0 | 0.0 |
| Lung ca. (small cell) NCI-H69 | 4.4 | 3.4 |
| Lung ca. (s. cell var.) SHP-77 | 0.2 | 0.0 |
| Lung ca. (large cell)NCI-H460 | 0.3 | 0.2 |
| Lung ca. (non-sm. cell) A549 | 0.0 | 0.0 |
| Lung ca. (non-s. cell) NCI-H23 | 0.8 | 0.7 |
| Lung ca (non-s. cell) HOP-62 | 4.2 | 10.2 |
| Lung ca. (non-s. cl) NCI-H522 | 0.0 | 0.2 |
| Lung ca. (squam.) SW 900 | 0.2 | 0.2 |
| Lung ca. (squam.) NCI-H596 | 8.2 | 13.5 |
| Mammary gland | 57.0 | 47.3 |
| Breast ca.* (pl. effusion) MCF-7 | 0.0 | 0.0 |
| Breast ca.* (pl. ef) MDA-MB-231 | 0.0 | 0.0 |
| Breast ca.* (pl. effusion) T47D | 0.0 | 0.1 |
| Breast ca. BT-549 | 7.3 | 7.5 |
| Breast ca. MDA-N | 0.0 | 0.0 |
| Ovary | 25.9 | 16.2 |
| Ovarian ca. OVCAR-3 | 2.5 | 1.7 |
| Ovarian ca. OVCAR-4 | 0.9 | 0.8 |
| Ovarian ca. OVCAR-5 | 0.2 | 0.2 |
| Ovarian ca. OVCAR-8 | 0.0 | 0.0 |
| Ovarian ca. IGROV-1 | 0.0 | 0.2 |
| Ovarian ca.* (ascites) SK-OV-3 | 0.4 | 0.7 |
| Uterus | 19.3 | 14.9 |
| Placenta | 53.2 | 27.2 |
| Prostate | 12.1 | 6.5 |
| Prostate ca.* (bone met)PC-3 | 6.3 | 6.8 |
| Testis | 5.6 | 3.7 |
| Melanoma Hs688(A).T | 96.6 | 100.0 |
| Melanoma* (met) Hs688(B).T | 29.7 | 28.5 |
| Melanoma UACC-62 | 0.8 | 1.4 |
| Melanoma M14 | 0.0 | 0.0 |
| Melanoma LOX IMVI | 0.4 | 0.2 |
| Melanoma* (met) SK-MEL-5 | 1.7 | 1.6 |
| Adipose | 57.0 | 40.6 |

TABLE BB
Panel 2.2

| | Relative |
|---|---|
| | Expression (%) |
| Tissue Name | 2.2x4tm6490t_ag1078_a1 |
| Normal Colon GENPAK | 18.0 |
| 061003 | |
| 97759 Colon cancer (OD06064) | 26.8 |
| 97760 Colon cancer NAT | 48.0 |
| (OD06064) | |
| 97778 Colon cancer (OD06159) | 2.8 |
| 97779 Colon cancer NAT | 23.7 |
| (OD06159) | |
| 98861 Colon cancer (OD06297-04) | 6.2 |
| 98862 Colon cancer NAT | 42.1 |
| (OD06297-015) | |
| 83237 CC Gr.2 ascend colon | 7.4 |
| (ODO3921) | |
| 83238 CC NAT (ODO3921) | 7.6 |
| 97766 Colon cancer metastasis | 2.5 |
| (OD06104) | |
| 97767 Lung NAT (OD06104) | 9.2 |
| 87472 Colon mets to lung | 2.1 |
| (OD04451-01) | |
| 87473 Lung NAT (OD04451-02) | 9.0 |
| Normal Prostate Clontech A + | 4.7 |
| 6546-1 (8090438) | |
| 84140 Prostate Cancer | 4.0 |
| (OD04410) | |
| 84141 Prostate NAT | 11.7 |
| (OD04410) | |
| Normal Ovary Res. Gen. | 10.2 |
| 98863 Ovarian cancer | 0.8 |
| (OD06283-03) | |
| 98865 Ovarian cancer | 18.9 |
| NAT/fallopian tube (OD06283-07) | |
| Ovarian Cancer GENPAK | 5.9 |
| 064008 | |
| 97773 Ovarian cancer | 2.2 |
| (OD06145) | |
| 97775 Ovarian cancer NAT | 20.1 |
| (OD06145) | |
| 98853 Ovarian cancer | 2.3 |
| (OD06455-03) | |
| 98854 Ovarian NAT | 18.0 |
| (OD06455-07) Fallopian tube | |
| Normal Lung GENPAK 061010 | 4.4 |
| 92337 Invasive poor diff. lung | 1.8 |
| adeno (ODO4945-01 | |
| 92338 Lung NAT (ODO4945-03) | 13.3 |
| 84136 Lung Malignant Cancer | 7.1 |
| (OD03126) | |
| 84137 Lung NAT (OD03126) | 3.7 |
| 90372 Lung Cancer | 2.5 |
| (OD05014A) | |
| 90373 Lung NAT (OD05014B) | 10.7 |
| 97761 Lung cancer (OD06081) | 0.0 |
| 97762 Lung cancer NAT | 7.9 |
| (OD06081) | |
| 85950 Lung Cancer (OD04237-01) | 0.6 |
| 85970 Lung NAT (OD04237-02) | 15.7 |
| 83255 Ocular Mel Met to Liver | 1.0 |
| (ODO4310) | |
| 83256 Liver NAT (ODO4310) | 1.7 |
| 84139 Melanoma Mets to Lung | 2.4 |
| (OD04321) | |
| 84138 Lung NAT (OD04321) | 15.9 |
| Normal Kidney GENPAK | 7.4 |
| 061008 | |
| 83786 Kidney Ca, Nuclear | 7.2 |
| grade 2 (OD04338) | |
| 83787 Kidney NAT (OD04338) | 0.0 |
| 83788 Kidney Ca Nuclear grade | 0.7 |
| ½ (OD04339) | |
| 83789 Kidney NAT (OD04339) | 5.7 |
| 83790 Kidney Ca, Clear cell | 3.3 |
| type (OD04340) | |
| 83791 Kidney NAT (OD04340) | 13.3 |
| 83792 Kidney Ca, Nuclear | 5.5 |
| grade 3 (OD04348) | |
| 83793 Kidney NAT (OD04348) | 30.1 |
| 98938 Kidney malignant cancer | 2.4 |
| (OD06204B) | |
| 98939 Kidney normal adjacent | 5.8 |
| tissue (OD06204E) | |
| 85973 Kidney Cancer | 0.0 |
| (OD04450-01) | |
| 85974 Kidney NAT (OD04450-03) | 4.7 |
| Kidney Cancer Clontech | 0.0 |
| 8120613 | |
| Kidney NAT Clontech 8120614 | 2.8 |
| Kidney Cancer Clontech | 0.3 |
| 9010320 | |
| Kidney NAT Clontech 9010321 | 1.3 |
| Kidney Cancer Clontech | 2.2 |
| 8120607 | |
| Kidney NAT Clontech 8120608 | 1.5 |
| Normal Uterus GENPAK | 100.0 |
| 061018 | |
| Uterus Cancer GENPAK | 11.9 |
| 064011 | |
| Normal Thyroid Clontech A + | 1.9 |
| 6570-1 (7080817) | |
| Thyroid Cancer GENPAK | 0.9 |
| 064010 | |
| Thyroid Cancer INVITROGEN | 5.9 |
| A302152 | |
| Thyroid NAT INVITROGEN | 3.7 |
| A302153 | |
| Normal Breast GENPAK | 54.2 |
| 061019 | |
| 84877 Breast Cancer | 1.2 |
| (OD04566) | |
| Breast Cancer Res. Gen. 1024 | 12.2 |
| 85975 Breast Cancer | 1.0 |
| (OD04590-01) | |
| 85976 Breast Cancer Mets | 20.2 |
| (OD04590-03) | |
| 87070 Breast Cancer Metastasis | 2.4 |
| (OD04655-05) | |
| GENPAK Breast Cancer | 9.3 |
| 064006 | |
| Breast Cancer Clontech | 10.3 |
| 9100266 | |
| Breast NAT Clontech 9100265 | 58.4 |
| Breast Cancer INVITROGEN | 9.8 |
| A209073 | |
| Breast NAT INVITROGEN | 27.7 |
| A2090734 | |
| 97763 Breast cancer | 12.1 |
| (OD06083) | |
| 97764 Breast cancer node | 4.5 |
| metastasis (OD06083) | |
| Normal Liver GENPAK | 4.8 |
| 061009 | |
| Liver Cancer Research | 0.0 |
| Genetics RNA 1026 | |
| Liver Cancer Research | 1.2 |
| Genetics RNA 1025 | |
| Paired Liver Cancer Tissue | 1.5 |
| Research Genetics RNA 6004-T | |
| Paired Liver Tissue Research | 0.3 |
| Genetics RNA 6004-N | |
| Paired Liver Cancer Tissue | 1.0 |
| Research Genetics RNA 6005-T | |
| Paired Liver Tissue Research | 4.2 |
| Genetics RNA 6005-N | |
| Liver Cancer GENPAK 064003 | 0.0 |
| Normal Bladder GENPAK | 1.5 |
| 061001 | |
| Bladder Cancer Research | 2.6 |
| Genetics RNA 1023 | |
| Bladder Cancer INVITROGEN | 1.5 |
| A302173 | |
| Normal Stomach GENPAK | 21.2 |
| 061017 | |
| Gastric Cancer Clontech | 3.7 |
| 9060397 | |
| NAT Stomach Clontech | 2.1 |
| 9060396 | |
| Gastric Cancer Clontech | 11.0 |
| 9060395 | |
| NAT Stomach Clontech | 14.6 |
| 9060394 | |
| Gastric Cancer GENPAK | 3.1 |
| 064005 | |

TABLE CC
Panel 4D

| | Relative |
|---|---|
| | Expression |
| | (%) |
| Tissue Name | 4dtm2546t_ag1078 |
| 93768_Secondary Th1_anti- | 0.0 |
| CD28/anti-CD3 | |
| 93769_Secondary Th2_anti- | 0.0 |
| CD28/anti-CD3 | |
| 93770_Secondary Tr1_anti- | 0.0 |
| CD28/anti-CD3 | |
| 93573_Secondary Th1_resting | 0.0 |
| day 4-6 in IL-2 | |
| 93572_Secondary Th2_resting | 0.0 |
| day 4-6 in IL-2 | |
| 93571_Secondary Tr1_resting | 0.0 |
| day 4-6 in IL-2 | |
| 93568_primary Th1_anti- | 0.0 |
| CD28/anti-CD3 | |
| 93569_primary Th2_anti- | 0.0 |
| CD28/anti-CD3 | |
| 93570_primary Tr1_anti- | 0.0 |
| CD28/anti-CD3 | |
| 93565_primary Th1_resting dy | 0.0 |
| 4-6 in IL-2 | |
| 93566_primary Th2_resting dy | 0.0 |
| 4-6 in IL-2 | |
| 93567_primary Tr1_resting dy | 0.0 |
| 4-6 in IL-2 | |
| 93351_CD45RA CD4 | 6.2 |
| lymphocyte_anti-CD28/anti- | |
| CD3 | |
| 93352_CD45RO CD4 | 0.0 |
| lymphocyte_anti-CD28/anti- | |
| CD3 | |
| 93251_CD8 Lymphocytes_anti- | 0.0 |
| CD28/anti-CD3 | |
| 93353_chronic CD8 | 0.0 |
| Lymphocytes 2ry_resting dy 4- | |
| 6 in IL-2 | |
| 93574_chronic CD8 | 0.0 |
| Lymphocytes 2ry_activated | |
| CD3/CD28 | |
| 93354_CD4_none | 0.0 |
| 93252_Secondary | 0.0 |
| Th1/Th2/Tr1_anti-CD95 CH11 | |
| 93103_LAK cells_resting | 0.0 |
| 93788_LAK cells_IL-2 | 0.0 |
| 93787_LAK cells_IL-2 + IL-12 | 0.0 |
| 93789_LAK cells_IL-2 + IFN | 0.0 |
| gamma | |
| 93790_LAK cells_IL-2 + IL-18 | 0.0 |
| 93104_LAK | 0.0 |
| cells_PMA/ionomycin and IL- | |
| 18 | |
| 93578_NK Cells IL-2_resting | 0.0 |
| 93109_Mixed Lymphocyte | 0.0 |
| Reaction_Two Way MLR | |
| 93110_Mixed Lymphocyte | 0.0 |
| Reaction_Two Way MLR | |
| 93111_Mixed Lymphocyte | 0.0 |
| Reaction_Two Way MLR | |
| 93112_Mononuclear Cells | 0.0 |
| (PBMCs)_resting | |
| 93113_Mononuclear Cells | 0.0 |
| (PBMCs)_PWM | |
| 93114_Mononuclear Cells | 0.0 |
| (PBMCs)_PHA-L | |
| 93249_Ramos (B cell)_none | 0.0 |
| 93250_Ramos (B | 0.0 |
| cell)_ionomycin | |
| 93349_B lymphocytes_PWM | 0.0 |
| 93350_B lymphoytes_CD40L | 0.0 |
| and IL-4 | |
| 92665_EOL-1 | 0.1 |
| (Eosinophil)_dbcAMP | |
| differentiated | |
| 93248_EOL-1 | 0.0 |
| (Eosinophil)dbcAMP/PMAion | |
| omycin | |
| 93356_Dendritic Cells_none | 0.0 |
| 93355_Dendritic Cells_LPS | 0.0 |
| 100 ng/ml | |
| 93775_Dendritic Cells_anti- | 0.0 |
| CD40 | |
| 93774_Monocytes_resting | 0.0 |
| 93776_Monocytes_LPS 50 | 0.0 |
| ng/ml | |
| 93581_Macrophages_resting | 0.0 |
| 93582_Macrophages_LPS 100 | 0.0 |
| ng/ml | |
| 93098_HUVEC | 0.3 |
| (Endothelial)_none | |
| 93099_HUVEC | 0.7 |
| (Endothelial)_starved | |
| 93100_HUVEC | 0.3 |
| (Endothelial)_IL-1b | |
| 93779_HUVEC | 0.9 |
| (Endothelial)_IFN gamma | 0.2 |
| 93102_HUVEC | |
| (Endothelial)_TNF alpha + IFN | 0.2 |
| gamma | |
| 93101_HUVEC | 0.2 |
| (Endothelial)_TNF alpha + IL4 | |
| 93781_HUVEC | 0.0 |
| (Endothelial)_IL-11 | |
| 93583_Lung Microvascular | 0.0 |
| Endothelial Cells_none | |
| 93584_Lung Microvascular | 0.0 |
| Endothelial Cells_TNFa (4 | |
| ng/ml) and IL1b (1 ng/ml) | |
| 92662_Microvascular Dermal | 0.0 |
| endothelium_none | |
| 92663_Microsvasular Dermal | 0.0 |
| endothelium_TNFa (4 ng/ml) | |
| and IL1b (1 ng/ml) | |
| 93773_Bronchial | 0.0 |
| epithelium_TNFa (4 ng/ml) and | |
| IL1b (1 ng/ml)** | |
| 93347_Small Airway | 1.1 |
| Epithelium_none | |
| 93348_Small Airway | 0.0 |
| Epithelium_TNFa (4 ng/ml) and | |
| IL1b (1 ng/ml) | |
| 92668_Coronery Artery | 1.4 |
| SMC_resting | |
| 92669_Coronery Artery | 0.7 |
| SMC_TNFa (4 ng/ml) and IL1b | |
| (1 ng/ml) | |
| 93107_astrocytes_resting | 0.3 |
| 93108_astrocytes_TNFa (4 | 0.0 |
| ng/ml) and IL1b (1 ng/ml) | |
| 92666_KU-812 | 0.0 |
| (Basophil)_resting | |
| 92667_KU-812 | 0.1 |
| (Basophil)_PMA/ionoycin | |
| 93579_CCD1106 | 0.0 |
| (Keratinocytes)_none | |
| 93580_CCD1106 | 0.0 |
| (Keratinocytes)_TNFa and IFNg | |
| ** | |
| 93791_Liver Cirrhosis | 0.9 |
| 93792_Lupus Kidney | 0.4 |
| 93577_NCI-H292 | 0.0 |
| 93358_NCI-H292_IL-4 | 0.0 |
| 93360_NCI-H292_IL-9 | 0.2 |
| 93359_NCI-H292_IL-13 | 0.0 |
| 93357_NCI-H292_IFN gamma | 0.2 |
| 93777_HPAEC_- | 0.0 |
| 93778_HPAEC_IL-1 beta/TNA | 0.0 |
| alpha | |
| 93254_Normal Human Lung | 29.9 |
| Fibroblast_none | |
| 93253_Normal Human Lung | 13.8 |
| Fibroblast_TNFa (4 ng/ml) and | |
| IL-lb (1 ng/ml) | |
| 93257_Normal Human Lung | 84.7 |
| Fibroblast_IL-4 | |
| 93256_Normal Human Lung | 30.6 |
| Fibroblast_IL-9 | |
| 93255_Normal Human Lung | 44.4 |
| Fibroblast_IL-13 | |
| 93258_Normal Human Lung | 100.0 |
| Fibroblast_IFN gamma | |
| 93106_Dermal Fibroblasts | 42.6 |
| CCD1070_resting | |
| 93361_Dermal Fibroblasts | 16.7 |
| CCD1070_TNF alpha 4 ng/ml | |
| 93105_Dermal Fibroblasts | 12.1 |
| CCD1070_IL-1 beta 1 ng/ml | |
| 93772_dermal fibroblast_IFN | 24.8 |
| gamma | |
| 93771_dermal fibroblast_IL-4 | 46.0 |
| 93259_IBD Colitis 1** | 0.7 |
| 93260_IBD Colitis 2 | 0.1 |
| 93261_IBD Crohns | 0.3 |
| 735010_Colon_normal | 5.3 |
| 735019_Lung_none | 11.9 |
| 64028-1_Thymus_none | 4.5 |
| 64030-1_Kidney_none | 2.6 |

Panel 1.2 Summary: The results obtained from the two separate RTQ-PCR experiments using Ag1078 are roughly in agreement. Expression of the NOV5 gene in this panel is largely restricted to normal tissues and two melanoma cell lines. This pattern is intriguing, especially with the inclusion of the melanoma cell lines, as in our experience, it is somewhat characteristic of genes expressed by endothelial cells. These observations are consistent with published reports that vascular endothelial-cadherin, an endothelial cell adhesion molecule, plays an essential role in the formation of stable and fully functional blood vessels (Cancer Metastasis Rev 2000;19:1-5). Furthermore, a monoclonal antibody to vascular endothelial-cadherin has been shown to be a potent inhibitor of angiogenesis, tumor growth, and metastasis (Cancer Res 2000; 60:6805-10). Thus, therapeutic modulation of the NOV5 gene may likewise be used to enhance or interfere with disease processes involving lack of or overabundance of angiogenesis respectively. Such diseases include, but are not limited to, cancer, cardiovascular disease and aberrant wound healing.

Panel 2.2 Summary: Expression of the NOV5 gene appears to be associated with normal tissues, when compared to their cancerous counterparts. This, coupled with the data in panel 1.2, might indicate that this gene is expressed in resting or non-activated endothelium. Thus, therapeutic modulation of this gene may be used to enhance or interfere with disease processes involving lack or overabundance of endothelial cells respectively. Such diseases include, but are not limited to, cancer, cardiovascular disease and aberrant wound healing. For example, hemangiomas are benign tumors of endothelium. Targeting of this gene in hemangioma may reverse this disease. Since tumors appear to lack the NOV5 transcript, the protein encoded by this gene may act as a tumor suppressor. Therefore, agonistic NOV5 protein therapeutics could be used to treat a variety of human tumors and to prevent metastasis.

Panel 4D Summary: NOV5 transcript expression is limited to fibroblasts. Expression of the transcript is not affected or is slightly increased by IL-4 or gamma interferon treatment and is reduced by IL-1 or TNF alpha treatment. The protein encoded for by this transcript could serve as a marker for fibroblasts. Additionally, agonistic protein therapeutics designed with the protein could reduce inflammation due to allergy, asthma, emphysema and bacterial infection, since all these conditions induce TNF alpha/IL-1 beta expression.

NOV6B

Expression of gene NOV6b was assessed using the primer-probe sets Ag2175, Ag2978, Ag2939, and Ag654 described in Tables DD, EE, and FF. Results of the RTQ-PCR runs are shown in Table GG and HH.

TABLE DD
Probe Name Ag2175

| | | | | | Start |
|---|---|---|---|---|---|
| Primers | Sequences | | TM | Length | Position |
| Forward | 5′-GCTCATTATGAGAGTGGCTTTG-3′ | (SEQ ID NO:128) | 59 | 22 | 467 |
| Probe | TET-5′-CTTCGTGGACCACAATCCTGATGG-3′-TAMRA | (SEQ ID NO:129) | 68.9 | 24 | 496 |
| Reverse | 5′-TCAGTTGGAAAATGCCATATTC-3′ | (SEQ ID NO:130) | 59 | 22 | 527 |

TABLE EE
Probe Name Ag2978 (equivalent to Ag2939)

| | | | | | Start |
|---|---|---|---|---|---|
| Primers | Sequences | | TM | Length | Position |
| Forward | 5′-CAATCGCCATATTCTGGATG-3′ | (SEQ ID NO:131) | 58.9 | 20 | 394 |
| Probe | 5′-AGAATGGGCTTTCTGCCTGGACTTCT-3′ | (SEQ ID NO:132) | 69.1 | 26 | 447 |
| Reverse | 5′-CCAGAACAGTGTAGCCTCCA3′ | (SEQ ID NO:133) | 58.9 | 20 | 473 |

TABLE FF
Probe Name Ag654

| | | | | Start |
|---|---|---|---|---|
| Primers | Sequences | TM | Length | Position |
| Forward | 5′-CTGTGCATGGCTCATTA | 58.2 | 20 | 81 |
| | TGA-3′ (SEQ ID NO: 134) | | | |
| Probe | FAM-5′-AGTGGCTTTGACA | 70.2 | 23 | 102 |
| | CCGCCTTCGT-3′-TAMRA | | | |
| | (SEQ ID NO: 135) | | | |
| Reverse | 5′-CATATTCACTGCTGCCA | 58.2 | 20 | 137 |
| | TCA-3′ (SEQ ID | | | |
| | NO: 136) | | | |

TABLE GG
Panel 1.3D

| | | | Relative |
|---|---|---|---|
| | Relative | Relative | Expression |
| | Expression (%) | Expression (%) | (%) |
| Tissue Name | 1.3dx4tm5487t_ag2175_a2 | 1.3dx4tm5283f_ag2978_b2 | 1.3dx4tm5494f_ag654_b2 |
| Liver adenocarcinoma | 0.0 | 0.0 | 0.0 |
| Pancreas | 5.3 | 0.0 | 1.9 |
| Pancreatic ca. CAPAN 2 | 0.9 | 0.0 | 2.3 |
| Adrenal gland | 1.6 | 0.0 | 1.9 |
| Thyroid | 3.7 | 0.0 | 3.6 |
| Salivary gland | 0.0 | 0.0 | 1.9 |
| Pituitary gland | 6.1 | 0.0 | 4.8 |
| Brain (fetal) | 6.3 | 0.0 | 1.6 |
| Brain (whole) | 2.8 | 0.0 | 4.0 |
| Brain (amygdala) | 3.1 | 1.4 | 4.5 |
| Brain (cerebellum) | 6.3 | 0.0 | 4.4 |
| Brain (hippocampus) | 4.9 | 0.2 | 8.9 |
| Brain (substantia nigra) | 3.1 | 0.4 | 3.7 |
| Brain (thalamus) | 12.6 | 1.8 | 2.0 |
| Cerebral Cortex | 1.7 | 0.5 | 0.0 |
| Spinal cord | 6.4 | 0.2 | 2.0 |
| CNS ca. (glio/astro) U87-MG | 0.0 | 0.0 | 0.0 |
| CNS ca. (glio/astro) U-118-MG | 0.0 | 0.0 | 0.0 |
| CNS ca. (astro) SW1783 | 0.0 | 0.0 | 0.0 |
| CNS ca.* (neuro; met) SK-N-AS | 0.0 | 0.0 | 0.0 |
| CNS ca. (astro) SF-539 | 1.0 | 0.0 | 0.0 |
| CNS ca. (astro) SNB-75 | 0.0 | 0.0 | 1.9 |
| CNS ca. (glio) SNB-19 | 1.1 | 0.0 | 0.7 |
| CNS ca. (glio) U251 | 5.7 | 0.0 | 0.8 |
| CNS ca. (glio) SF-295 | 2.7 | 0.0 | 0.6 |
| Heart (fetal) | 0.0 | 0.0 | 0.0 |
| Heart | 1.3 | 0.0 | 1.8 |
| Fetal Skeletal | 2.1 | 0.0 | 1.1 |
| Skeletal muscle | 1.1 | 0.0 | 0.0 |
| Bone marrow | 3.0 | 0.0 | 1.9 |
| Thymus | 4.5 | 2.7 | 4.0 |
| Spleen | 2.8 | 0.0 | 0.0 |
| Lymph node | 7.6 | 0.3 | 5.1 |
| Colorectal | 3.0 | 1.1 | 2.2 |
| Stomach | 9.9 | 2.7 | 3.4 |
| Small intestine | 6.0 | 0.0 | 4.8 |
| Colon ca. SW480 | 0.0 | 1.3 | 0.0 |
| Colon ca.* (SW480 met)SW620 | 0.0 | 0.0 | 0.0 |
| Colon ca. HT29 | 0.0 | 0.0 | 0.0 |
| Colon ca. HCT-116 | 0.0 | 0.0 | 0.0 |
| Colon ca. CaCo-2 | 1.0 | 0.0 | 0.7 |
| 83219 CC Well to Mod Diff | 0.0 | 0.0 | 0.0 |
| (ODO3866) | | | |
| Colon ca. HCC-2998 | 0.0 | 0.0 | 0.0 |
| Gastric ca.* (liver met) NCI-N87 | 3.7 | 0.0 | 1.8 |
| Bladder | 1.7 | 1.2 | 4.3 |
| Trachea | 0.0 | 0.0 | 4.3 |
| Kidney | 1.8 | 0.0 | 1.8 |
| Kidney (fetal) | 3.0 | 0.0 | 0.0 |
| Renal ca. 786-0 | 0.0 | 0.0 | 0.0 |
| Renal ca. A498 | 1.8 | 0.0 | 1.7 |
| Renal ca. RXF 393 | 0.0 | 0.0 | 0.7 |
| Renal ca. ACHN | 0.0 | 0.0 | 0.0 |
| Renal ca. UO-31 | 1.1 | 0.0 | 0.0 |
| Renal ca. TK-10 | 1.6 | 0.0 | 0.0 |
| Liver | 1.0 | 0.0 | 0.0 |
| Liver (fetal) | 0.0 | 0.0 | 0.0 |
| Liver ca. (hepatoblast) HepG2 | 0.0 | 0.0 | 0.0 |
| Lung | 2.0 | 1.4 | 1.0 |
| Lung (fetal) | 0.9 | 0.0 | 1.0 |
| Lung ca. (small cell) LX-1 | 1.1 | 0.0 | 2.9 |
| Lung ca. (small cell) NCI-H69 | 0.0 | 0.0 | 0.0 |
| Lung ca. (s. cell var.) SHP-77 | 0.0 | 0.0 | 0.0 |
| Lung ca. (large cell) NCI-H460 | 0.0 | 0.0 | 1.4 |
| Lung ca. (non-sm. cell) A549 | 0.0 | 0.0 | 0.0 |
| Lung ca. (non-s. cell) NCI-H23 | 3.6 | 0.0 | 3.1 |
| Lung ca (non-s. cell) HOP-62 | 1.7 | 0.0 | 0.7 |
| Lung ca. (non-s. cl) NCI-H522 | 1.2 | 0.0 | 0.0 |
| Lung ca. (squam.) SW 900 | 0.0 | 0.0 | 0.0 |
| Lung ca. (squam.) NCI-H596 | 0.0 | 0.0 | 0.0 |
| Mammary gland | 4.2 | 0.0 | 0.0 |
| Breast ca.* (pl. effusion) MCF-7 | 2.6 | 0.0 | 1.2 |
| Breast ca.* (pl. ef) MDA-MB-231 | 1.9 | 0.0 | 0.0 |
| Breast ca.* (pl. effusion) T47D | 0.7 | 1.3 | 0.8 |
| Breast ca. BT-549 | 1.4 | 0.0 | 0.0 |
| Breast ca. MDA-N | 0.0 | 0.0 | 0.0 |
| Ovary | 0.0 | 0.0 | 0.0 |
| Ovarian Ca. OVCAR-3 | 1.0 | 0.0 | 1.8 |
| Ovarian Ca. OVCAR-4 | 0.7 | 0.0 | 0.0 |
| Ovarian Ca. OVCAR-5 | 0.0 | 0.0 | 1.6 |
| Ovarian ca. OVCAR-8 | 0.0 | 0.0 | 2.9 |
| Ovarian ca. IGROV-1 | 1.1 | 0.0 | 0.0 |
| Ovarian ca.* (ascites) SK-OV-3 | 0.0 | 0.0 | 0.0 |
| Uterus | 6.0 | 0.0 | 3.6 |
| Placenta | 0.8 | 0.0 | 0.0 |
| Prostate | 5.5 | 1.7 | 4.3 |
| Prostate ca.* (bone met)PC-3 | 1.2 | 0.0 | 0.0 |
| Testis | 100.0 | 100.0 | 100.0 |
| Melanoma Hs688(A).T | 0.0 | 0.0 | 0.0 |
| Melanoma* (met) Hs688(B).T | 1.4 | 0.0 | 0.0 |
| Melanoma UACC-62 | 0.0 | 0.0 | 0.0 |
| Melanoma M14 | 0.0 | 0.0 | 0.0 |
| Melanoma LOX IMVI | 0.0 | 0.0 | 0.0 |
| Melanoma* (met) SK-MEL-5 | 0.0 | 0.0 | 0.0 |
| Adipose | 1.1 | 0.0 | 1.5 |

TABLE HH
Panel 4D

| | Relative | Relative |
|---|---|---|
| | Expression | Expression |
| | (%) | (%) |
| | 4dtm4704f_ | 4dx4tm5494f_ |
| Tissue Name | ag2939 | ag654 |
| 93768_Secondary Th1_anti-CD28/anti-CD3 | 0.0 | 0.0 |
| 93769_Secondary Th2_anti-CD28/anti-CD3 | 0.0 | 0.0 |
| 93770_Secondary Tr1_anti-CD28/anti-CD3 | 0.0 | 0.0 |
| 93573_Secondary Th1_resting day 4-6 in IL-2 | 15.8 | 0.0 |
| 93572_Secondary Th2_resting day 4-6 in IL-2 | 13.8 | 8.1 |
| 93571_Secondary Tr1_resting day 4-6 in IL-2 | 9.4 | 5.9 |
| 93568_primary Th1_anti-CD28/anti-CD3 | 0.0 | 17.4 |
| 93569_primary Th2_anti-CD28/anti-CD3 | 0.0 | 0.0 |
| 93570_primary Tr1_anti-CD28/anti-CD3 | 8.0 | 5.6 |
| 93565_primary Th1_resting dy 4-6 in IL-2 | 95.3 | 36.3 |
| 93566_primary Th2_resting dy 4-6 in IL-2 | 25.5 | 32.5 |
| 93567_primary Tr1_resting dy 4-6 in IL-2 | 50.3 | 20.2 |
| 93351_CD45RA CD4 lymphocyte_anti-CD28/anti-CD3 | 0.0 | 0.0 |
| 93352_CD45RO CD4 lymphocyte_anti-CD28/anti-CD3 | 80.7 | 4.0 |
| 93251_CD8 Lymphocytes_anti-CD28/anti-CD3 | 14.1 | 7.2 |
| 93353_chronic CD8 Lymphocytes 2ry_resting dy 4-6 in IL-2 | 40.9 | 0.0 |
| 93574_chronic CD8 Lymphocytes 2ry_activated CD3/CD28 | 0.0 | 0.0 |
| 93354_CD4_none | 33.2 | 7.3 |
| 93252_Secondary Th1/Th2/Tr1_anti-CD95 CH11 | 0.0 | 7.4 |
| 93103_LAK cells_resting | 0.0 | 16.0 |
| 93788_LAK cells_IL-2 | 0.0 | 8.7 |
| 93787_LAK cells_IL-2 + IL-12 | 37.4 | 19.8 |
| 93789_LAK cells_IL-2 + IFN gamma | 76.8 | 22.5 |
| 93790_LAK cells_IL-2 + IL-18 | 0.0 | 12.7 |
| 93104_LAK cells_PMA/ionomycin and IL-18 | 0.0 | 0.0 |
| 93578_NK Cells IL-2_resting | 24.1 | 10.2 |
| 93109_Mixed Lymphocyte Reaction_Two Way MLR | 23.2 | 9.7 |
| 93110_Mixed Lymphocyte Reaction_Two Way MLR | 15.1 | 4.1 |
| 93111_Mixed Lymphocyte Reaction_Two Way MLR | 70.2 | 3.2 |
| 93112_Mononuclear Cells (PBMCs)_resting | 28.1 | 12.9 |
| 93113_Mononuclear Cells (PBMCs)_PWM | 23.3 | 15.2 |
| 93114_Mononuclear Cells (PBMCs)_PHA-L | 0.0 | 15.6 |
| 93249_Ramos (B cell)_none | 9.7 | 15.8 |
| 93250_Ramos (B cell)_ionomycin | 75.8 | 26.1 |
| 93349_B lymphocytes_PWM | 47.6 | 0.0 |
| 93350_B lymphoytes_CD40L and IL-4 | 35.8 | 35.8 |
| 92665_EOL-1 (Eosinophil)_dbcAMP differentiated | 0.0 | 0.0 |
| 93248_EOL-1 (Eosinophil)_dbcAMP/PMA ionomycin | 0.0 | 0.0 |
| 93356_Dendritic Cells_none | 0.0 | 8.6 |
| 93355_Dendritic Cells_LPS 100 ng/ml | 10.5 | 2.8 |
| 93775_Dendritic Cells_anti-CD40 | 12.5 | 3.6 |
| 93774_Monocytes_resting | 17.2 | 13.0 |
| 93776_Monocytes_LPS 50 ng/ml | 0.0 | 5.4 |
| 93581_Macrophages_resting | 97.9 | 23.2 |
| 93582_Macrophages_LPS 100 ng/ml | 0.0 | 2.5 |
| 93098_HUVEC (Endothelial)_none | 0.0 | 5.5 |
| 93099_HUVEC (Endothelial)_starved | 0.0 | 0.7 |
| 93100_HUVEC (Endothelial)_IL-1b | 0.0 | 3.3 |
| 93779_HUVEC (Endothelial)_IFN gamma | 39.5 | 4.5 |
| 93102_HUVEC (Endothelial)_TNF alpha + IFN gamma | 0.0 | 0.0 |
| 93101_HUVEC (Endothelial)_TNF alpha + IL4 | 0.0 | 0.0 |
| 93781_HUVEC (Endothelial)_IL-11 | 13.4 | 16.5 |
| 93583_Lung Microvascular Endothelial Cells_none | 16.6 | 6.2 |
| 93584_Lung Microvascular Endothelial Cells_TNFa (4 | 38.7 | 10.6 |
| ng/ml) and IL1b (1 ng/ml) | | |
| 92662_Microvascular Dermal endothelium_none | 0.0 | 8.4 |
| 92663_Microsvasular Dermal endothelium_TNFa (4 ng/ml) | 0.0 | 14.5 |
| and IL1b (1 ng/ml) | | |
| 93773_Bronchial epithelium_TNFa (4 ng/ml) and IL1b (1 | 0.0 | 0.0 |
| ng/ml)** | | |
| 93347_Small Airway Epithelium_none | 0.0 | 0.9 |
| 93348_Small Airway Epithelium_TNFa (4 ng/ml) and IL1b | 76.8 | 19.6 |
| (1 ng/ml) | | |
| 92668_Coronery Artery SMC_resting | 0.0 | 0.0 |
| 92669_Coronery Artery SMC_TNFa (4 ng/ml) and IL1b (1 | 0.0 | 0.0 |
| ng/ml) | | |
| 93107_astrocytes_resting | 0.0 | 8.5 |
| 93108_astrocytes_TNFa (4 ng/ml) and IL1b (1 ng/ml) | 13.6 | 1.7 |
| 92666_KU-812 (Basophil)_resting | 0.0 | 0.0 |
| 92667_KU-812 (Basophil)_PMA/ionoycin | 0.0 | 0.0 |
| 93579_CCD1106 (Keratinocytes)_none | 0.0 | 5.5 |
| 93580_CCD1106 (Keratinocytes)_TNFa and IFNg** | 0.0 | 27.5 |
| 93791_Liver Cirrhosis | 9.0 | 61.6 |
| 93792_Lupus Kidney | 29.7 | 49.0 |
| 93577_NCI-H292 | 69.7 | 24.7 |
| 93358_NCI-H292_IL-4 | 7.2 | 26.8 |
| 93360_NCI-H292_IL-9 | 0.0 | 1.6 |
| 93359_NCI-H292_IL-13 | 0.0 | 0.0 |
| 93357_NCI-H292_IFN gamma | 8.3 | 3.7 |
| 93777_HPAEC_- | 13.0 | 0.0 |
| 93778_HPAEC_IL-1 beta/TNA alpha | 0.0 | 1.5 |
| 93254_Normal Human Lung Fibroblast_none | 0.0 | 3.0 |
| 93253_Normal Human Lung Fibroblast_TNFa (4 ng/ml) and | 21.0 | 0.0 |
| IL-1b (1 ng/ml) | | |
| 93257_Normal Human Lung Fibroblast_IL-4 | 0.0 | 2.1 |
| 93256_Normal Human Lung Fibroblast_IL-9 | 0.0 | 0.0 |
| 93255_Normal Human Lung Fibroblast_IL-13 | 0.0 | 1.6 |
| 93258_Normal Human Lung Fibroblast_IFN gamma | 0.0 | 0.0 |
| 93106_Dermal Fibroblasts CCD1070_resting | 0.0 | 2.0 |
| 93361_Dermal Fibroblasts CCD1070_TNF alpha 4 ng/ml | 0.0 | 5.1 |
| 93105_Dermal Fibroblasts CCD1070_IL-1 beta 1 ng/ml | 0.0 | 0.0 |
| 93772_dermal fibroblast_IFN gamma | 0.0 | 12.7 |
| 93771_dermal fibroblast_IL-4 | 8.1 | 0.0 |
| 93259_IBD Colitis 1** | 0.0 | 100.0 |
| 93260_IBD Colitis 2 | 0.0 | 1.9 |
| 93261_IBD Crohns | 0.0 | 2.0 |
| 735010_Colon_normal | 24.7 | 12.7 |
| 735019_Lung_none | 46.0 | 1.8 |
| 64028-1_Thymus_none | 100.0 | 100.0 |
| 64030-1_Kidney_none | 69.3 | 42.0 |

Panel 1.3D Summary: The results obtained from three separate RTQ-PCR experiments using different probe and primer sets are roughly in agreement. Expression of the NOV6b gene appears to be largely restricted to testis and this gene could therefore be used to identify/differentiate testes from other tissues. Very low levels of the NOV6b transcript are also detected in the thymus and brain, consistent with the results obtained in panel 4D.

Panel 2.2 Summary: Expression of the NOV6b gene in panel 2.2 was low/undetectable (Ct values>35) in all samples.

Panel 4D Summary: For Ag654, significant expression of the NOV6b transcript is detected in the thymus, in primary resting Tr1/Th1 and Th2 cells, and in B cells treated with CD40L and IL-4. Expression in IBD colitis 1 is probably due to genomic contamination. This molecule may be important in T cell development in the thymus, T cell and B cell differentiation, and B cell isotype switching. Regulation of this molecule by small molecule therapeutics could function to regulate immunity and be important for tissue transplantation, vaccine development and treatment of autoimmune diseases. For Ag2939, all samples had CT values>35; however, the greatest level of expression was again seen in the thymus.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS:1, 3, 5, 7, 9, 11, 13, 15, 16, 17, 19, 21, 23, 25, 27 or a complement of this aforementioned nucleotide sequence, can be isolated using standard molecular biology techniques and the sequence information provided herein. Using all or a portion of the nucleic acid sequence of SEQ ID NOS:1, 3, 5, 7, 9, 11, 13, 15, 16, 17, 19, 21, 23, 25, 27 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS:1, 3, 5, 7, 9, 11, 13, 15, 16, 17, 19, 21, 23, 25, and 27, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7, 9, 11, 13, 15, 16, 17, 19, 21, 23, 25, and 27, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS:1, 3, 5, 7, 9, 11, 13, 15, 16, 17, 19, 21, 23, 25, or 27 is one that is sufficiently complementary to the nucleotide sequence shown SEQ ID NOS:1, 3, 5, 7, 9, 11, 13, 15, 16, 17, 19, 21, 23, 25, or 27 that it can hydrogen bond with little or no mismatches to the nucleotide sequence shown SEQ ID NOS:1, 3, 5, 7, 9, 11, 13, 15, 16, 17, 19, 21, 23, 25, and 27, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS:1, 3, 5, 7, 9, 11, 13, 15, 16, 17, 19, 21, 23, 25, and 27, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS:1, 3, 5, 7, 9, 11, 13, 15, 16, 17, 19, 21, 23, 25, or 27; or an anti-sense strand nucleotide sequence of SEQ ID NOS:1, 3, 5, 7, 9, 11, 13, 15, 16, 17, 19, 21, 23, 25, or 27; or of a naturally occurring mutant of SEQ ID NOS:1, 3, 5, 7, 9, 11, 13, 15, 17, 19, 21, 23, 25, and 27.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS:1, 3, 5, 7, 9, 11, 13, 15, 16, 17, 19, 21, 23, 25, or 27 that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS:1, 3, 5, 7, 9, 11, 13, 15, 16, 17, 19, 21, 23, 25, and 27 due to degeneracy of the genetic code and thus encode the same NOVX proteins as that encoded by the nucleotide sequences shown in SEQ ID NOS:1, 3, 5, 7, 9, 11, 13, 15, 16, 17, 19, 21, 23, 25, and 27. In another embodiment, an isolated nucleic acid molecule of the invention has a nucleotide sequence encoding a protein having an amino acid sequence shown in SEQ ID NOS:2, 4, 6, 8, 10, 12, 14, 18, 20, 22, 24, 26, and 28.

In addition to the human NOVX nucleotide sequences shown in SEQ ID NOS:1, 3, 5, 7, 9, 11, 13, 15, 16, 17, 19, 21, 23, 25, and 27, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS:1, 3, 5, 7, 9, 11, 13, 15, 16, 17, 19, 21, 23, 25, and 27 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7, 9, 11, 13, 15, 16, 17, 19, 21, 23, 25, and 27. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, el al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6X SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2X SSC, 0.01% BSA at 50° C. An isolated nucleic acid molecule of the invention that hybridizes under stringent conditions to the sequences SEQ ID NOS:1, 3, 5, 7, 9, 11, 13, 15, 16, 17, 19, 21, 23, 25, and 27, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 16, 17, 19, 21, 23, 25, and 27, or fragments, analogs or derivatives thereof, under conditions of moderate stringency is provided. A non-limiting example of moderate stringency hybridization conditions are hybridization in 6X SSC, 5X Denhardt's solution, 0.5% SDS and 100 mg/ml denatured salmon sperm DNA at 55° C., followed by one or more washes in 1X SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS:1, 3, 5, 7, 9, 11, 13, 15, 16, 17, 19, 21, 23, 25, and 27, or fragments, analogs or derivatives thereof, under conditions of low stringency, is provided. A non-limiting example of low stringency hybridization conditions are hybridization in 35% formamide, 5X SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2X SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS:1, 3, 5, 7, 9, 11, 13, 15, 16, 17, 19, 21, 23, 25, and 27, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2, 4, 6, 8, 10, 12, 14, 18, 20, 22, 24, 26, and 28. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS:2, 4, 6, 8, 10, 12, 14, 18, 20, 22, 24, 26, or 28 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2, 4, 6, 8, 10, 12, 14, 18, 20, 22, 24, 26, or 28. Preferably, the protein encoded by the nucleic acid molecule is at least about 60% homologous to SEQ ID NOS:2, 4, 6, 8, 10, 12, 14, 18, 20, 22, 24, 26, or 28; more preferably at least about 70% homologous SEQ ID NOS:2, 4, 6, 8, 10, 12, 14, 18, 20, 22, 24,26, and 28; still more preferably at least about 80% homologous to SEQ ID NOS:2, 4, 6, 8, 10, 12, 14, 18, 20, 22, 24, 26, and 28; even more preferably at least about 90% homologous to SEQ ID NOS:2, 4, 6, 8, 10, 12, 14, 18, 20, 22, 24, 26, and 28; and most preferably at least about 95% homologous to SEQ ID NOS:2, 4, 6, 8, 10, 12, 14, 18, 20, 22, 24, 26, and 28.

An isolated nucleic acid molecule encoding an NOVX protein homologous to the protein of SEQ ID NOS:2, 4, 6, 8, 10, 12, 14, 18, 20, 22, 24, 26, and 28 can be created by introducing one or more nucleotide substitutions, additions or deletions into the nucleotide sequence of SEQ ID NOS:1, 3, 5, 7, 9, 11, 13, 15, 16, 17, 19, 21, 23, 25, and 27, such that one or more amino acid substitutions, additions or deletions are introduced into the encoded protein.

Mutations can be introduced into SEQ ID NOS:2, 4, 6, 8, 10, 12, 14, 18, 20, 22, 24, 26, and 28 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS:1, 3, 5, 7, 9, 11, 13, 15, 16, 17, 19, 21, 23, 25, and 27, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 16, 17, 19, 21, 23, 25, and 27, or fragments, analogs or derivatives thereof. An “antisense” nucleic acid comprises a nucleotide sequence that is complementary to a “sense” nucleic acid encoding a protein (e.g., complementary to the coding strand of a double-stranded CDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 4, 6, 8, 10, 12, 14, 18, 20, 22, 24, 26, and 28, or antisense nucleic acids complementary to an NOVX nucleic acid sequence of SEQ ID NOS:1, 3, 5, 7, 9, 11, 13, 15, 16, 17, 19, 21, 23, 25, and 27, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracit, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS:1, 3, 5, 7, 9, 11, 13, 15, 16, 17, 19, 21, 23, 25, and 27). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (see, Hyrup, et al., 1996.supra); or as probes or primers for DNA sequence and hybridization (see, Hyrup, et al., 1996, supra; Perry-O'Keefe, et al, 1996.supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See. e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2, 4, 6, 8, 10, 12, 14, 18, 20, 22, 24, 26, and 28. The invention also includes a mutant or variant protein any of whose residues may be changed from the corresponding residues shown in SEQ ID NOS:2, 4, 6, 8, 10, 12, 14, 18, 20, 22, 24, 26, and 28 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, ie., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2, 4, 6, 8, 10, 12, 14, 18, 20, 22, 24, 26, and 28) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2, 4, 6, 8, 10, 12, 14, 18, 20, 22, 24, 26, and 28. In other embodiments, the NOVX protein is substantially homologous to SEQ ID NOS:2, 4, 6, 8, 10, 12, 14, 18, 20, 22, 24, 26, and 28, and retains the functional activity of the protein of SEQ ID NOS:2, 4, 6, 8, 10, 12, 14, 18, 20, 22, 24, 26, and 28, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2, 4, 6, 8, 10, 12, 14, 18, 20, 22, 24, 26, and 28, and retains the functional activity of the NOVX proteins of SEQ ID NOS:2, 4, 6, 8, 10, 12, 14, 18, 20, 22, 24, 26, and 28.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 5, 7, 9, 11, 13, 15, 16, 17, 19, 21, 23, 25, and 27.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS:2, 4, 6, 8, 10, 12, 14, 18, 20, 22, 24, 26, and 28), whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et at. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with SI nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci USA 89: 7811-7815; Delgrave, et al., 1993. Protein Engineering 6:327-331.

Anti-NOVX Antibodies

The invention encompasses antibodies and antibody fragments, such as F_{ab} or (F_{ab})₂, that bind immunospecifically to any of the NOVX polypeptides of said invention.

An isolated NOVX protein, or a portion or fragment thereof, can be used as an immunogen to generate antibodies that bind to NOVX polypeptides using standard techniques for polyclonal and monoclonal antibody preparation. The full-length NOVX proteins can be used or, alternatively, the invention provides antigenic peptide fragments of NOVX proteins for use as immunogens. The antigenic NOVX peptides comprises at least 4 amino acid residues of the amino acid sequence shown SEQ ID NOS:2, 4, 6, 8, 10, 12, 14, 18, 20, 22, 24, 26, and 28 and encompasses an epitope of NOVX such that an antibody raised against the peptide forms a specific immune complex with NOVX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of NOVX that is located on the surface of the protein (e.g., a hydrophilic region). As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation (see, e.g., Hopp and Woods, 1981. Proc. Nat. Acad. Sci. USA 78: 3824-3828; Kyte and Doolittle, 1982. J. Mol. Biol. 157: 105-142, each incorporated herein by reference in their entirety).

As disclosed herein, NOVX protein sequences of SEQ ID NOS:2, 4, 6, 8, 10, 12, 14, 18, 20, 22, 24, 26, 28, or derivatives, fragments, analogs or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as NOVX. Such antibodies include, but are not limited to, polyclonal, monoclonal, chimeric, single chain, F_{ab} and F(_{ab})₂ fragments, and an F_{ab} expression library. In a specific embodiment, antibodies to human NOVX proteins are disclosed. Various procedures known within the art may be used for the production of polyclonal or monoclonal antibodies to an NOVX protein sequence of SEQ ID NOS:2, 4, 6, 8, 10, 12, 14, 18, 20, 22, 24, 26, 28, or a derivative, fragment, analog or homolog thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed NOVX protein or a chemically-synthesized NOVX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against NOVX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of NOVX. A monoclonal antibody composition thus typically displays a single binding affinity for a particular NOVX protein with which it immunoreacts. For preparation of monoclonal antibodies directed towards a particular NOVX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to an NOVX protein (see, e.g., U.S. Pat. No. 4,946,778). In addition, methods can be adapted for the construction of F_{ab} expression libraries (see, e.g., Huse, et al., 1989. Science 246: 1275-1281) to allow rapid and effective identification of monoclonal F_{ab} fragments with the desired specificity for an NOVX protein or derivatives, fragments, analogs or homologs thereof. Non-human antibodies can be “humanized” by techniques well known in the art. See, e.g., U.S. Pat. No. 5,225,539. Antibody fragments that contain the idiotypes to an NOVX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent; and (iv) Fᵥ fragments.

Additionally, recombinant anti-NOVX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314 :446-449; Shaw, et al., 1988. J. Natl. Cancer Inst. 80: 1553-1559); Morrison(1985) Science 229:1202-1207; Oi, et al. (1986) BioTechniques 4:214; Jones, et al., 1986. Nature 321: 552-525; Verhoevan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No.4,873,316 and European Application Publication No.264,166). Developmentally-regulated promoters are also encompassed, e.g., the murine hox promoters (Kessel and Gruss, 1990. Science 249: 374-379) and the co-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,”Reviews-Trends in Genetics, Vol.1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et at. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS:1, 3, 5, 7, 9, 11, 13, 15, 16, 17, 19, 21, 23, 25, and 27 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS:1, 3, 5, 7, 9, 11, 13, 15, 16, 17, 19, 21, 23, 25, and 27), but more preferably, is a non-human homologue of a human NOVX gene. For example, a mouse homologue of human NOVX gene of SEQ ID NOS:1, 3, 5, 7, 9, 11, 13, 15, 16, 17, 19, 21, 23, 25, and 27 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdernal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, stipra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl Acad. Sci U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl--N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS:1, 3, 5, 7, 9, 11, 13, 15, 16, 17, 19, 21, 23, 25, and 27, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g.. in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 16, 17, 19, 21, 23, 25, and 27 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.) Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:1, 3, 5, 7, 9, 11, 13, 15, 16, 17, 19, 21, 23, 25, and 27, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Nati. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, ie., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators ( ie., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g. from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid Molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i e., some peptides have been found to possess anti-bacterial properties). These materials are further useful in the generation of antibodies which immunospecifically-bind to the novel substances of the invention for use in therapeutic or diagnostic methods.

### EQUIVALENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 18, 20, 22, 24, 26, and 28;
(b) a variant of a mature form of an amino acid sequence selected from the group consisting of SEQ ID NOS:2, 4, 6, 8, 10, 12, 14, 18, 20, 22, 24, 26, and 28, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS:2, 4, 6, 8, 10, 12, 14, 18, 20, 22, 24, 26, and 28; and
(d) a variant of an amino acid sequence selected from the group consisting of SEQ ID NOS:2, 4, 6, 8, 10, 12, 14, 18, 20, 22, 24, 26, and 28, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting SEQ ID NOS:2, 4, 6, 8, 10, 12, 14, 18, 20, 22, 24, 26, and 28.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS:1, 3, 5, 7, 9, 11, 13, 15, 16, 17, 19, 21, 23, 25, and 27.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 10, 12, 14, 18, 20, 22, 24, 26, and 28;
(b) a variant of a mature form of an amino acid sequence selected from the group consisting of SEQ ID NOS:2, 4, 6, 8, 10, 12, 14, 18, 20, 22, 24, 26, and 28, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 18, 20, 22, 24, 26, and 28;
(d) a variant of an amino acid sequence selected from the group consisting SEQ ID NOS:2, 4, 6, 8, 10, 12, 14, 18, 20, 22, 24, 26, and 28, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 18, 20, 22, 24, 26, and 28, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 16, 17, 19, 21, 23, 25, and 27.

9. The nucleic acid molecule of claim 5, wherein said nucleic acid molecule comprises a nucleotide sequence selected from the group consisting of:
(a) a nucleotide sequence selected from the group consisting of SEQ ID NOS:1, 3, 5, 7, 9, 11, 13, 15, 16, 17, 19, 21, 23, 25, and 27;
(b) a nucleotide sequence differing by one or more nucleotides from a nucleotide sequence selected from the group consisting of SEQ ID NOS:1, 3, 5, 7, 9, 11, 13, 15, 16, 17, 19, 21, 23, 25, and 27, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting SEQ ID NOS:1, 3, 5, 7, 9, 11, 13, 15, 16, 17, 19, 21, 23, 25, and 27, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule, thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26; wherein said subject is a human.

28. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

29. The method of claim 28, wherein said subject is a human.

30. A method of treating or preventing a NOVX-associated disorder, said method comprising administering to a subject in which such treatment or prevention is desired the antibody of claim 15 in an amount sufficient to treat or prevent said NOVX-associated disorder in said subject.

31. The method of claim 30, wherein the subject is a human.

32. A pharmaceutical composition comprising the polypeptide of claim 1 and a pharmaceutically-acceptable carrier.

33. A pharmaceutical composition comprising the nucleic acid molecule of claim 5 and a pharmaceutically-acceptable carrier.

34. A pharmaceutical composition comprising the antibody of claim 15 and a pharmaceutically-acceptable carrier.

35. A kit comprising in one or more containers, the pharmaceutical composition of claim 32.

36. A kit comprising in one or more containers, the pharmaceutical composition of claim 33.

37. A kit comprising in one or more containers, the pharmaceutical composition of claim 34.

38. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

39. The method of claim 38 wherein the predisposition is to cancers.

40. A method for determining the presence of or predisposition to a disease associated with altered levels of the nucleic acid molecule of claims in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

41. The method of claim 40 wherein the predisposition is to a cancer.

42. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 18, 20, 22, 24, 26, and 28, or a biologically active fragment thereof.

43. A method of treating a pathological state in a mammal, the method comprising administering to the mammal the antibody of claim 15 in an amount sufficient to alleviate the pathological state.
